(19) United States
(12) Patent Application Publication
(10) Pub. No.: US 2026/0177549 A1
(43) Pub. Date: Jun. 25, 2026

(54) METHODS, DIAGNOSTIC INSTRUMENTS, AND KITS FOR DETECTING DISEASES

(71) Applicant: Kephera Diagnostics, LLC, Framingham, MA (US)

(72) Inventor: Andrew E. Levin, Wellesley, MA (US)

(73) Assignee: Kephera Diagnostics, LLC, Framingham, MA (US)

(21) Appl. No.: 19/127,588

(22) PCT Filed: Nov. 7, 2023
(86) PCT No.: PCT/US2023/078966

§ 371 Date: May 6, 2025

Related U.S. Application Data
(60) Provisional application No. 63/423,199, filed on Nov. 7, 2022.

(51) Int. Cl. G01N 33/543

(52) U.S. Cl. CPC G01N 33/54388

(57) ABSTRACT

Provided herein are methods, diagnostic instruments, and kits for detecting the presence or absence of an analyte associated with a disease in a subject. In some embodiments, the disease is Lyme disease, SARS-CoV-2, or a human immunodeficiency virus infection.

### CROSS-REFERENCE TO RELATED APPLICATIONS

This application claims the benefit under 35 U.S.C. § 119(e) of U.S. Provisional Application No. 63/423,199, filed Nov. 7, 2022, entitled “METHODS, DIAGNOSTIC INSTRUMENTS, AND KITS FOR DETECTING DISEASES,” the entire disclosure of which is hereby incorporated by reference in its entirety.

### FIELD OF THE INVENTION

The present disclosure relates to methods, systems, compositions, diagnostic instruments, and kits related to improved detection of diseases.

### BACKGROUND

Early detection of infectious diseases, such as Lyme disease, COVID-19, and HIV, has the potential to reduce the number of illnesses and/or deaths per year and improve outcomes. However, many existing detection methods are time-consuming, expensive, and laborious. Accordingly, improved methods and systems for the detection of infectious diseases are needed.

### SUMMARY OF THE INVENTION

The present disclosure relates, at least in part, to methods, systems, compositions, kits, and uses for detection and diagnosis of the presence or recent presence of a disease in a subject.

Certain aspects of the present disclosure relate to a method of detecting the presence or absence of an analyte in a sample, the method comprising: contacting the sample with a first binding molecule bound to a detectable label and incubating the sample with the first binding molecule; contacting the sample with a second binding molecule bound to a magnetic particle and incubating the sample with the second binding molecule; adding the sample to a capillary vessel comprising an elongated capillary body having a first end and a second end disposed distal the first end, a sample entry port proximal the first end of the capillary body, and a sample exit port distal the first end of the capillary body and proximal the second end of the capillary body; and assessing the presence of a signal from the detectable label within the capillary vessel; wherein: a magnetic field is applied to at least a portion of the capillary vessel; the presence of a signal within the capillary vessel is indicative of association of the analyte derived from the sample with the first binding molecule and the second binding molecule; and incubating the first binding molecule and the second binding molecule with the sample is sufficient to bind the first binding molecule to a first binding site on the analyte and the second binding molecule to a second binding site on the analyte.

Certain aspects of the present disclosure relate to a diagnostic instrument adapted to receive a capillary vessel comprising an elongated capillary body having a first end and a second end disposed distal the first end, a sample entry port proximal the first end of the capillary body, and a sample exit port distal the first end of the capillary body and proximal the second end of the capillary body; wherein the diagnostic instrument further comprises: means for application of a magnetic field to a portion of the capillary body between the sample entry port and the sample exit port; and means for detection of a signal in a portion of the capillary body between the sample entry port and the sample exit port.

Certain aspects of the present disclosure relate to a diagnostic instrument, comprising: a capillary vessel comprising an elongated capillary body having a first end and a second end disposed distal the first end, a sample entry port proximal the first end of the capillary body, and a sample exit port distal the first end of the capillary body and proximal the second end of the capillary body; means for application of a magnetic field to a portion of the capillary body between the sample entry port and the sample exit port; and means for detection of a signal in a portion of the capillary body between the sample entry port and the sample exit port.

Certain aspects of the present disclosure relate to a kit, comprising: a first reagent comprising a first binding molecule bound to a detectable label; and a second reagent comprising a second binding molecule bound to a magnetic particle.

Certain aspects of the present disclosure relate to a kit, comprising: a first reagent comprising a first binding molecule bound to a detectable label; a second reagent comprising a second binding molecule bound to a magnetic particle; and a capillary pad.

Certain aspects of the present disclosure relate to a kit, comprising: a first reagent comprising a first binding molecule bound to a detectable label; a second reagent comprising a second binding molecule bound to a magnetic particle; and a capillary vessel comprising an elongated capillary body having a first end and a second end disposed distal the first end, a sample entry port proximal the first end of the capillary body, and a sample exit port distal the first end of the capillary body and proximal the second end of the capillary body.

Certain aspects of the present disclosure relate to a kit, comprising: a first reagent comprising a detectable label adapted to be bound to a first binding molecule; a second reagent comprising a magnetic particle adapted to be bound to a second binding molecule; a capillary vessel comprising an elongated capillary body having a first end and a second end disposed distal the first end, a sample entry port proximal the first end of the capillary body, and a sample exit port distal the first end of the capillary body and proximal the second end of the capillary body.

Certain aspects of the present disclosure relate to a kit, comprising a capillary vessel comprising an elongated capillary body having a first end and a second end disposed distal the first end, a sample entry port proximal the first end of the capillary body, and a sample exit port distal the first end of the capillary body and proximal the second end of the capillary body; wherein: the capillary vessel is adapted to interface with a diagnostic instrument comprising: means for application of a magnetic field to a portion of the capillary body between the sample entry port and the sample exit port; and means for detection of a signal in a portion of the capillary body between the sample entry port and the sample exit port.

A housing assembly, comprising: a top portion; and a bottom portion removably attached to the top portion; wherein: the top portion is adapted to receive one or more cameras and one or more excitation sources; the bottom portion is adapted to receive a magnet; and the bottom portion is adapted to receive a diagnostic instrument.

The details of one or more embodiments of the present disclosure are set forth in the description below. Other features or advantages of the present invention will be apparent from the following drawings and detailed description of several embodiments, and also from the appended claims.

### BRIEF DESCRIPTION OF THE DRAWINGS

The accompanying drawings are not necessarily drawn to scale. For purposes of clarity, not every component may be labeled in every drawing. In the drawings:

FIG. 1 shows a photograph of exemplary high volume lateral flow production equipment.

FIG. 2 shows an exemplary schematic detailing the steps and timing of one embodiment of Magnetic Capture Rapid Assay (MiCRA; hereafter referred to as Magnetic Capture Rapid ImmunoAssay (MiCRIA)) as described herein.

FIG. 3 shows an exemplary schematic of the structure and components of one embodiment of MiCRIA as described herein as compared to lateral flow devices.

FIG. 4 shows a schematic of an exemplary rounded rectangular glass capillary vessel that can be used in certain MiCRIA assays disclosed herein.

FIG. 5 shows a photograph of an exemplary capillary assembly as disclosed herein.

FIG. 6 shows a photograph of an exemplary MiCRIA assay device as described herein.

FIG. 7 shows a photograph of an exemplary magnetic capture in a capillary channel as described herein.

FIGS. 8A-8F show photographs of an exemplary MiCRIA assay device as described herein wherein initiation of liquid flow through a capillary channel is affected by horizontal displacement of a sealing pad and adsorbent pad over the capillary exit port.

FIGS. 9A-9E show photographs of an exemplary MiCRIA assay device as described herein wherein initiation of liquid flow through a capillary channel is affected by horizontal displacement of a sealing pad and adsorbent pad over the capillary exit port.

FIGS. 10A-10B show photographs of an exemplary reader assembly as described herein.

FIGS. 11A-11H show photographs of exemplary detection of Lyme disease serum samples and negative control serum samples by MiCRIA as described herein.

FIGS. 12A-12E show photographs of exemplary results from testing of serum samples from Lyme disease positive patients by MiCRIA as described herein.

FIGS. 13A-13E show photographs of exemplary results from testing of serum samples from Lyme disease negative patients by MiCRIA as described herein.

FIGS. 14A-14B show exemplary Area Under the Curve (AUC) calculation results for the samples shown in FIGS. 12-13 detected by MiCRIA as described herein.

FIGS. 15A-15E show photographs of results of an exemplary MiCRIA assay used for detection of antibodies in whole blood samples spiked with Lyme disease positive and Lyme disease negative serum samples as described herein.

FIG. 16 shows results of an exemplary MiCRIA assay using a mixture that was incubated for 1 minute and a mixture that was not incubated prior to being loaded onto the MiCRIA instrument, as described herein.

FIGS. 17A-17B show photographs of results of an exemplary MiCRIA assay used for detection of dried whole blood samples spiked with Lyme disease positive and Lyme disease negative samples, as described herein.

FIG. 18 shows a photograph of an exemplary detection of HIV-positive (left) and -negative (right) sera by MiCRIA as described herein.

FIG. 19 shows a graph of exemplary MiCRIA signal intensity compared to the dilution factor for an HIV positive serum sample.

FIGS. 20A-20B show photographs of results of an exemplary MiCRIA assay used for detection of HIV-positive samples and HIV-negative samples, as described herein.

FIG. 21 shows a graphical representation of quantitative measurement of HIV-positive and HIV-negative samples as detected by exemplary MiCRIA assays, as described herein.

FIGS. 22A-22B show photographs of results of an exemplary MiCRIA assay used for detection of antibodies to the COVID-19 Receptor Binding Domain, as described herein.

FIGS. 23A-23D show photographs of results of an exemplary MiCRIA assay used for detection of antibodies to a peptide antigen derived from the COVID-19 spike protein in serum samples from COVID-19 patients, as described herein.

FIGS. 24A-24D show photographs of results of an exemplary MiCRIA assay used for detection of antibodies to a peptide antigen derived from the COVID-19 spike protein in serum samples from uninfected negative controls, as described herein.

FIGS. 25A-25B show photographs of results of an exemplary MiCRIA assay used for detection of antibodies to a peptide derived from the COVID-19 nucleocapsid protein in serum samples from a COVID-19 patient (FIG. 25B) and a negative control (FIG. 25A) (FIG. 25A, negative result; FIG. 25B, positive result), as described herein.

FIGS. 26A-26J show detection of the COVID-19 nucleocapsid antigen by MiCRIA as described herein. Each panel shows a visualization of the signal detected at 60 seconds (top) and a quantification of the signal detected at various timepoints (bottom) when using 1 ng of nucleocapsid (FIGS. 26A-28B), 0.1 ng of nucleocapsid (FIGS. 26C-26D), 0.01 ng of nucleocapsid (FIGS. 26E-26F), 0.001 ng of nucleocapsid (FIGS. 26G-26H), or 0 ng of nucleocapsid (FIGS. 26I-26J) spiked in a buffer solution.

FIG. 27 shows a chart of the quantitative measurements of the MiCRIA signal obtained in testing serial dilutions of COVID-19 nucleocapsid antigen shown in FIGS. 26A-26J in an exemplary MiCRIA assay, as described herein.

FIGS. 28A-28E show the sensitivity of an exemplary MiCRIA assay in detecting COVID-19 antibodies, as described herein.

FIGS. 29A-29B show a comparison of the sensitivity of an exemplary ELISA assay in detecting COVID-19 antibodies and the MiCRIA assay in detecting COVID-19 antibodies, as described herein.

FIG. 30 shows an exemplary MiCRIA assay assembly, as described herein.

FIG. 31 shows an exploded top view of the exemplary MiCRIA assay assembly of FIG. 30. as described herein.

FIG. 32 shows an exploded perspective view of the exemplary MiCRIA assay assembly of FIG. 30, with the capillary body inserted through the rubber septum, as described herein.

FIG. 33 shows an exploded perspective view of the exemplary MiCRIA assay assembly of FIG. 30, as described herein.

FIG. 34 shows a perspective drawing of an exemplary MiCRIA housing assembly, as described herein.

FIGS. 35A-35C show additional drawings of the exemplary MiCRIA housing assembly of FIG. 34, as described herein, with FIG. 35A showing a side view, FIG. 35B showing a front view, and FIG. 35C showing a top view.

FIG. 36 shows an exploded perspective drawing of the exemplary MiCRIA housing assembly of FIG. 34, as described herein.

FIG. 37 shows an exploded side perspective drawing of the exemplary MiCRIA housing assembly of FIG. 34 with assembled sub-components, as described herein.

FIG. 38 shows a perspective drawing of the exemplary MiCRIA housing assembly of FIG. 34 with a transparent lid, as described herein.

FIGS. 39A-39B show full exploded drawings of the exemplary MiCRIA housing assembly of FIG. 34, with either one (FIG. 39A) or two (FIG. 39B) excitation sources, as described herein.

FIGS. 40A-40B show perspective transparent drawings of the MiCRIA housing assembly of FIG. 34 with either one (FIG. 40A) or two (FIG. 40B) excitation sources, as described herein.

FIG. 41 shows a comparison of the sensitivity of the MiCRIA assay, a lateral flow assay, and an ELISA assay in detecting the presence of spiked-in Cs1-specifical monoclonal antibody (4E11) in negative serum samples, as described herein.

FIGS. 42A-42B shows a comparison of sensitivity and specificity of the MiCRIA assay and an ELISA assay in detecting the presence of antibodies derived from Chagas disease-positive and Chagas disease-negative samples. FIG. 42A shows the sensitivity and specificity of the MiCRIA assay in detecting the presence of antibodies derived from Chagas disease-positive and Chagas disease-negative samples. FIG. 42B shows the sensitivity and specificity of an ELISA assay in detecting the presence of antibodies derived from Chagas disease-positive and Chagas disease-negative samples.

FIG. 43 shows a comparison of the sensitivity of the MiCRIA assay and an ELISA assay in detecting the presence of anti-RBD IgG in SARS-CoV-2-positive samples, as described herein.

FIG. 44 shows a comparison of the sensitivity of the MiCRIA assay and a lateral flow assay in detecting the presence of anti-C6 peptide IgG in Lyme Disease-positive samples, as described herein.

FIG. 45 shows a comparison of the sensitivity of the MiCRIA assay and a lateral flow assay in detecting the presence of 4E11 in Liver Fluke-positive samples, as described herein.

FIG. 46 shows a comparison of the sensitivity of the MiCRIA assay and an ELISA assay in detecting the presence of anti-RBD IgG in SARS-CoV-2-positive samples, as described herein.

### DETAILED DESCRIPTION

The field of point-of-care testing for diagnosis of various medical conditions has undergone intense growth in recent years. As a result, the user base for diagnostic tests has moved progressively upstream, from the laboratory to the clinician to the patient. This movement has both democratized the field of diagnostic testing by spreading it to a much larger customer base and, in many cases, reduced healthcare costs.

Lateral flow, now in its third decade, has been the dominant technology in the global point-of-care testing market. While relatively simple to use, it is the opposite to create and produce, due to its high complexity and the multiple material components, labor, specialized training, controlled environment, and expensive dedicated equipment needed. As a result, development and manufacture of lateral flow tests is a specialty endeavor that can be carried out only by organizations well-endowed with the necessary resources. Alternatively, subcontractors have arisen that provide custom lateral flow development services, but at high cost.

Development of a traditional lateral flow test involves selecting a variety of components including membrane material, colloidal or other particle as label, sample pad, conjugate pad, sink pad, backing card, cassette housing and potential add-ons such as cover tape and blood separation media. Development then requires iterative optimization of this combination of components with various permutations of buffers and reagents. An antigen or antibody must be incorporated into all test strips. Serological lateral flow assays are significantly harder to develop than antigen capture assays, but in both cases, the number of different components and procedures to be optimized results in a complex assay development path that is often subject to setbacks, delays, and other obstacles. Despite the assertions, lateral flow development is not a “plug and play” endeavor and success generally requires prior experience and multiple iterations of assay development.

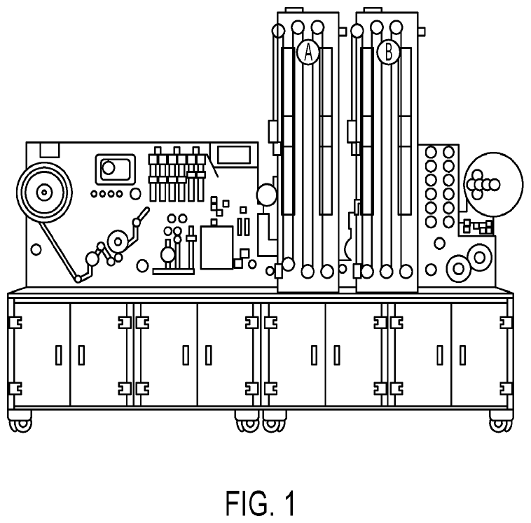

Following development, manufacturing of lateral flow tests poses additional challenges. High volume manufacture of lateral flow tests requires reel-to-reel equipment to stripe antigens or antibodies onto nitrocellulose membranes (e.g., using equipment similar to that shown in FIG. 1), for which the cost is in the hundreds of thousands of dollars and up, setting a high bar for anyone who wishes to produce a lateral flow test. Consequently, many smaller organizations opt to subcontract production of tests to companies that are already equipped with the requisite instrumentation, but the relatively small number of such companies creates a bottleneck and adds cost to the process. Many high-volume lateral flow manufacturing companies are based in Asia, where labor rates are lower than in the U.S., but outsourcing to Asia brings other challenges including ensuring consistent quality and preventing theft of intellectual property. The process of transferring a newly developed lateral flow test from R&D to a manufacturing facility can take weeks to months. While low-volume production can be accomplished with a card-based lateral flow dispensing instrument and manual packaging of test strips in cassettes, the throughput is relatively low without expensive automation. The requirements for producing lateral flow kits are significantly more demanding than for producing ELISA kits. Lateral flow strips are also sensitive to humidity, requiring a humidity-controlled environment for manufacturing.

The net result of these combined requirements and constraints is that in vitro diagnostics developers face significant challenges in time, cost and logistics in bringing a lateral flow test from development to manufacture and commercial launch. Moreover, the cost of manufacture may not be justified by the market size for many lateral flow tests which address specialty conditions or esoteric diseases. Lastly, the production of lateral flow tests is a multi-step procedure which can take a week or more per batch, as the test strip is built up from multiple components which must undergo final assembly into the typical cassette housing, another highly exacting process. These factors in sum represent a barrier to the rapid availability of point-of-care diagnostic tests for newly emerging pathogens, and especially a handicap to smaller assay developers who may have strong scientific capabilities but not the equipment and infrastructure for lateral flow test production. These issues have been highlighted during the most recent surge of the COVID-19 Omicron variant, during which an acute shortage of lateral flow rapid antigen tests was experienced worldwide, notwithstanding the efforts of the major industrial players in the market including Abbott, Roche and Becton Dickinson. In fact, FDA was obligated to change its policy for Emergency Use Authorization (EUA) approval of such tests, stating that it would only accept EUA applications from organizations that could demonstrate the capability for high volume production once approved.

These factors underscore the need for alternative point-of-care technologies that offer a simpler, faster and more straightforward assay development process, and more streamlined and cost-effective manufacturing scale-up than lateral flow assays. While many sophisticated chip-based point-of-care technologies are in development, these are generally expensive and involve complex manufacturing processes and instrumentation; on the other hand, there are few if any simple, low-cost, non-electronic options.

Existing lateral flow tests depend on the flow of test solutions along a nitrocellulose membrane, past an immobilized ligand or ligands (“immunochromatography”). Target analytes which bind to the immobilized ligand are detected by a reporter, commonly via particles bearing visual or fluorescent labels. Aside from acting as a solid substrate on which a ligand can be immobilized, the membrane does not contribute to the separation of bound from free target molecule—its major function is rather to channel the flow of liquid containing the target past the immobilized ligand. There is no intrinsic requirement for a membrane to carry out this function. In addition to the membrane, lateral flow devices comprise a collection of other paper or plastic components, including sample pad, conjugate pad, sink pad, backing card, sometimes cover tape, and typically a plastic cassette housing. In reality, the simplicity of the lateral flow method as perceived by the end user conceals a highly complex development and manufacturing procedure, which adds time and cost and makes lateral flow assay development a specialty subject requiring expensive dedicated instrumentation and corresponding skill sets.

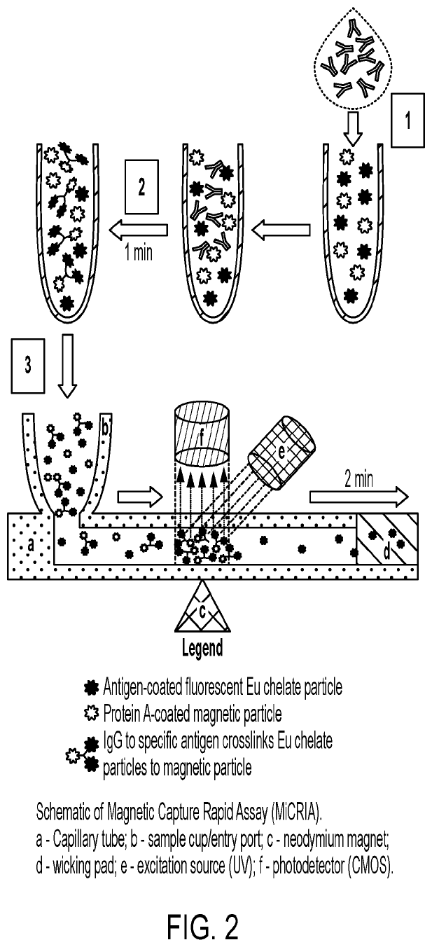

The present disclosure relates to MiCRIA, based on a novel assay principle which bypasses the problems of lateral flow development and manufacture. MiCRIA enables the separation of bound from free target analyte carried out in a liquid capillary channel via a magnetic capture principle, unlike the conventional immunochromatographic approach. As a consequence of the separation step being carried out in a liquid capillary channel, the assay time can also be significantly reduced, from the 15-20 minutes typical of lateral flow assays to 2 minutes in the MiCRIA assay. In the exemplary embodiment shown in FIG. 2, the MiCRIA technology can be implemented using the following steps: (1) a sample (e.g., serum or whole blood) suspected of containing an analyte is added to a mixture of a first binding molecule bound to a detectable label (e.g., antigen-coated Europium chelate beads) and a second binding molecule bound to a magnetic particle (Protein A-coated magnetic beads); (2) the mixture is incubated briefly, allowing immune complexes to form, then (3) the mixture is introduced to a capillary vessel ([a])for separation and detection. Because immobilization of bound analyte is accomplished through application of a magnetic field (generated by magnet [c]), there is no need to print or attach a capture reagent to the assay device itself, as in the test line on a lateral flow strip. Consequently, the MiCRIA capillary can be produced as a generic device, suitable for use in any MiCRIA test. Assay specificity resides in the particle components which can easily be tailored to the application of interest. While lateral flow and ELISA assays must be individually customized for the detection of different antigens, MiCRIA has the additional advantage of utilizing the same instrumentation, with variation only needed in consumables that can be provided in low-cost kits. In addition, MiCRIA is faster and cheaper than lateral flow and ELISA, requiring significantly less complex and more readily available materials, and offers better sensitivity and comparable sensitivity to lateral flow and ELISA, respectively. Overall, MiCRIA is a simple process due, at least in part, to the simplicity of the bead conjugation approach, thus enabling a test for a wide variety of different antigens that can be brought to a wider audience due to the lack of requirement for specialized skills and laboratory resources that are typically required to develop and deploy rapid diagnostics tests. MiCRIA thus provides an elegant and affordable solution for rapid diagnostics that can be deployed even in locations with simple laboratory resources. Accordingly, the present disclosure accordingly provides a novel point-of-care assay technology that offers the advantages of a straightforward development path with only one variable component to optimize, utilizing a generic consumable, with potential for near-limitless scalability in a short time.

In more detail, step 1 of the exemplary embodiment show in FIG. 2 shows a sample (e.g., whole blood, dried blood spot eluate, or serum) comprising target molecules (e.g., antibodies or analytes to be detected) being added to a vessel containing a first binding molecule bound to a detectable label (e.g., a fluorescent particle coated with a specific ligand, e.g., the antigenic target of the antibody that is to be detected in the sample, e.g., antigen-coated Europium chelate beads) and a second binding molecule bound to a magnetic particle (e.g., coated with a second binding agent or ligand such as Protein A/G that enables binding to the antibody Fc region, e.g., Protein A-coated magnetic beads). In step 2, the mixture is incubated for approximately 1 minute to allow binding of antibodies to antigens and formation of resultant antibody-particle complexes. Incubation of the sample with the first and second binding molecules can be performed either simultaneously or sequentially. This incubation step can be carried out in a vessel such as a test tube, or in the reservoir [b]. In some embodiments, the volume of incubation is relatively small (e.g., 50 microliters or less), reducing the diffusion barrier that can slow down the rate of binding. This short incubation allows antibodies to rapidly and efficiently bind both the first and second binding molecules. In step 3, the resultant mixture is then introduced into a reservoir ([b]) fluidically coupled to a sample entry port of a capillary vessel ([a]), such as by the use of a disposable pipette (e.g., a plastic or glass pipette). A magnet ([c]) is positioned at a specific point under and between the entry and exit ports of the capillary vessel. A chase buffer is optionally added, and the reactant mixture flows through the capillary vessel and is absorbed by the capillary pad (e.g., a wicking pad) [d] at the exit port of the capillary vessel. In some embodiments, the chase buffer is a Tris-based buffer, an MES-based buffer, a phosphate-base buffer, or phosphate buffered saline. In some embodiments, a surfactant selected from TWEEN® 20 and Triton X-100 is used. As the mixture flows through the capillary vessel, the magnetic field generated by the magnet captures the magnetic particles and any optical reporters that are crosslinked to them via the bound target molecules in a narrow band consistent with the location of the magnet, while unbound materials continue to flow through and are absorbed by a capillary pad ([d]) at the exit port. The chase buffer washes unbound material out of the capillary, leaving only magnetic beads and bound reporter captured at approximately the midpoint of the capillary by the magnet. In some embodiments, the optical signal is read visually or by a fluorescence sensor ([f]). The resulting signal (e.g., fluorescent Europium) is detected by the sensor and transmitted to a user interface.

In some embodiments, the MiCRIA assay disclosed herein is based, at least in part, on the intrinsic ability of molecules with multiple binding valencies or specificities to cross-link a magnetic particle to an optical reporter particle or other form of label, where both are derivatized with ligands recognized by the target molecule.

Magnetic particles, including paramagnetic particles, have been used in many immunological assays as capture tools, commonly to capture antibodies or antigens, and particles derivatized with various ligands such as Protein A, streptavidin, etc. for easy conjugation are commercially widely available (e.g., Cytiva, ThermoFisher, etc.). Magnetic capture in order to detect an optical signal has been described in three other immunoassay applications, but in ELISA-like tube formats (Burbelo et al. Sci Rep. 2017; 7(1):1-11; Kim et al., ACS Sensors. 2017 Jun. 23; 2(6):766-72; Mechaly et al., Anal Chem 2016 June 21; 88(12):6283-91). Paramagnetic particles have also been used as labels in lateral flow approaches in which sensitive magnetometers measure the magnetic signal as an indicator of the amount of target analyte captured to the membrane (Handali, et al. Clin Vaccine Immunol 2010 April; 17(4):631-7). However, the inventors believe the MiCRIA technology described herein to be the first to use paramagnetic particles as a means to capture an optical signal in a rapid test device in which the particles, including those to which an optical label is bound via specific antibody-antigen interaction, are captured magnetically as they flow through a capillary channel. In exemplary embodiments, MiCRIA is applied to multiple assay types including both immunoassays and hybridization-based molecular assays. MiCRIA thus offers a platform technology suitable for development of a wide variety of rapid tests for diverse applications.

One exemplary difference in both the principle and mechanism of MiCRIA vs. lateral flow is in the binding interaction between the assay probe and the target molecule. In the typical lateral flow assay, binding takes place as the target in the sample flows over the immobilized probe, such that binding is governed by kinetics rather than equilibrium. In contrast, in a MiCRIA assay, the probe and target are first mixed, permitting binding under conditions closer to equilibrium (governed by incubation time), after which bound and free target molecules are separated, resulting in higher sensitivity.

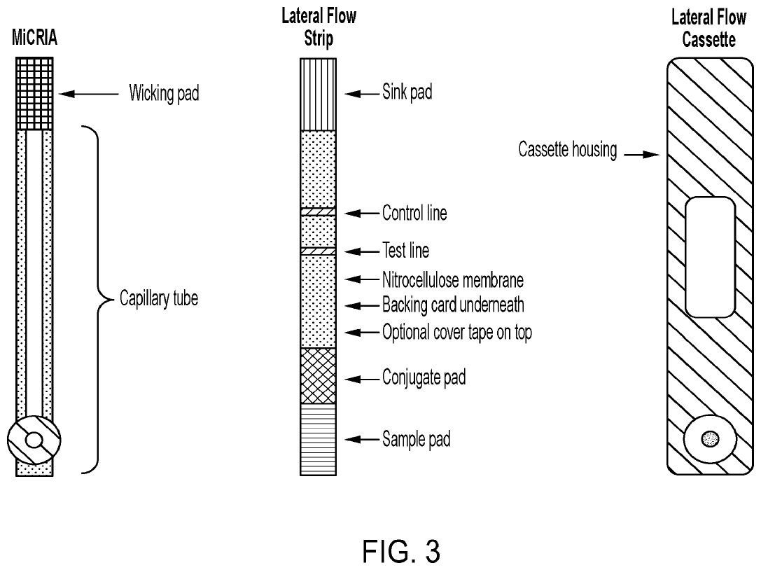

The MiCRIA assays disclosed herein do not require a membrane and in some embodiments are implemented using only a capillary vessel with capillary pad. As shown in FIG. 3, in one embodiment, MiCRIA (left in FIG. 3) includes only two components while lateral flow devices (center and right in FIG. 3) typically have six or more components, which require careful assembly. In some embodiments, a MiCRIA assay disclosed herein entails manufacture of only two assay-specific components: (1) Paramagnetic particles coated with one ligand, and (2) Reporter particles or molecules conjugated to a second ligand. In some embodiments, coating is accomplished by passive adsorption or by chemical crosslinking. In one exemplary embodiment, Protein A/G-coated paramagnetic particles are used to bind antibodies at the Fc region, while the reporter particles are coated with the target antigen by passive adsorption, biotin-streptavidin, or by chemical crosslinking using e.g., EDC and techniques known in the art (Crosslinking Technology: Reactivity chemistries, applications and structure references; Crosslinker Selection Guide; Fischer, In Humana Press; 2010 p. 55-73; Covalent Coupling). Using commercial Protein A/G paramagnetic particles, there is only one step involved in creating a new antibody detection assay—coating the optical reporter, e.g., fluorescent Europium latex particles, with target antigen. This advantage enables manufacturing MiCRIA assays as a bulk process, with particle coating scaled up readily by orders of magnitude in a basic laboratory with simple equipment such as a stirrer, centrifuge, and optionally an ultrasonic probe to disperse particles. In some embodiments utilizing commercial Europium particles, 1 μL of coated particles per assay is sufficient, enabling production of one million assays by coating 1 Liter of particles with antigen, a task which a lab technician could be expected to perform in a day. Thereafter, test kits are produced by packaging of the particle reagents and a chase buffer in dropper bottles (as are typically used in point-of-care test kits). Liquid dispensing equipment for this purpose is relatively inexpensive and readily available (e.g., Hamilton, Dispense Works, etc.).

In some embodiments, a MiCRIA assay utilizes a generic capillary vessel as a consumable, and a reusable reader containing the capture magnet. In embodiments utilizing a visual rather than fluorescent reporter, the reader is simplified to provide only the magnetic capture function and allow for reading by eye. In some embodiments, the capillary vessel with entry port is injection molded in volume in a clear, UV-transparent plastic such as acrylic. In other embodiments, glass capillaries are used. In some embodiments, each vessel incorporates a capillary pad as the only additional component. In some embodiments, the reader device incorporates a light source and a photodiode or CMOS sensor. In some embodiments utilizing fluorescent Europium reporters, the light source is a 365 nm ultraviolet LED. In some embodiments, the reader device is positioned opposite a magnet. In some embodiments, the magnet is an ultra-small, high-strength neodymium magnet. In some embodiments, the capillary vessel is inserted between the magnet and optical components. In some embodiments, the sensor signal is quantitated and provided to a user interface. In some embodiments, the user interface is disposed on the reader. In some embodiments, the user interface is disposed on a wirelessly connected device such as a Smartphone or Tablet computer. In some embodiments, the reader comprises a small footprint, is handheld, and/or is not substantially larger than the capillary vessel itself.

In some embodiments, the cost of the assay reagents, including magnetic and optical reporter particles, is less than $0.32 per assay in low volumes, decreasing significantly with scale up. This is in part because the magnets utilized in accordance with the present disclosure are small compared to those used in other assays. The flexibility to use small magnets has important impacts on the design and cost of a commercial reader device for MiCRIA assays, as neodymium magnets of 2 mm×1 mm size can be sourced commercially in large quantities at less than $0.02 each. The cost of a generic, molded capillary vessel is estimated at several cents in quantity. The optical reader device is projected to cost less than about $50 per unit when scaled up, and will be a one-time purchase usable over many assays. As recent precedents, Ellume marketed a SARS-CoV-2 rapid antigen test incorporating a fluorescent reader as a disposable home use product for about $40, and Lucira currently markets a similar one-time molecular test for SARS-CoV-2 for home use at $75. With the total cost per test at scale anticipated to be ≤$1.00 for consumables, MiCRIA is positioned to compete successfully with lateral flow in global point-of-care testing markets.

The MiCRIA assays disclosed herein place the tools and materials for point-of-care assay development at the disposal of laboratories and personnel without specialized training and equipment, and at a price point competitive with lateral flow. This democratizes and decentralizes point-of-care assay development, allowing organizations beyond the established diagnostics players to carry out the development and production of tests in-house at low cost, with concomitant benefit to the healthcare community.

In distinction from other rapid and microfluidic assays, the binding of the analyte to the binding molecules is substantially accomplished prior to introduction into the capillary, followed by a rapid separation step carried out within the capillary. Binding can thus be carried out under conditions much closer to true equilibrium conditions, unlike in lateral flow and other assays where binding takes place under kinetic, not equilibrium conditions as antibodies or analytes travel down the membrane or microfluidic channel and bind to a ligand immobilized on the membrane or the channel. Enabling binding under equilibrium conditions enhances or maximizes the concentration of bound antibody or analyte, resulting in higher sensitivity of detection than is possible where binding occurs under kinetic, non-equilibrium conditions, as in lateral flow. The relatively low volume in which incubations take place, along with the use of samples and reagents at low or no dilution, minimizes diffusion barriers that are responsible for the longer incubation times associated with typical immunoassays, such as ELISAs.

In further distinction from other rapid and microfluidic assays, in some embodiments the magnet is incorporated within an external, re-usable device that also incorporates the reader elements (e.g., a UV light source and camera or photodiode), and is not attached to the capillary. Alternatively, in some embodiments, the magnet is attached to the capillary as an integral part of the capillary device.

In further distinction from other rapid assays such as typical lateral flow assays, in some embodiments no ligand and/or binding agent is immobilized on the capillary device itself. In some embodiments, the capillary device is therefore manufactured as a generic unit for use with any MiCRIA assay. The MiCRIA assays disclosed herein therefore enable efficient scale up of the manufacture of the capillary device as a generic, consumable product without having to incorporate processes to add and immobilize biological reagents.

The present disclosure provides methods, systems, compositions, kits, and uses for improved detection and diagnosis of the presence or recent presence of a disease in a subject. In addition to providing dramatically increased simplicity as compared with other assay formats (such as lateral flow), the present disclosure allows for significantly faster turnaround times as compared to other available assays. Faster assays mean that more patients can be tested in the same amount of time, providing a significant advantage where there is a need to screen a large number of people efficiently (e.g., screening students in a school or residents in a nursing home). The onset of the COVID-19 pandemic and the overwhelming need for testing as occurred in 2020 and 2021 provides a case in point. Rapid testing for COVID-19 was largely carried out with lateral flow tests, which generally take 15-20 minutes each to provide results. In contrast, the present disclosure allows for test results in a much shorter time (e.g., 2 minutes)—roughly an order of magnitude faster. These time savings are compounded exponentially when considering increasing numbers of samples to be tested. In addition, a more rapid test allows medical decisions to be made more promptly (e.g., a 2-minute test would enable a clinician to test a patient within the typical 15 minute office visit, leaving adequate time to use the results to determine a diagnosis, potentially suggest other tests as needed, and inform the treatment plan). In the setting of an emergency room or clinic, an incoming patient could be tested for diseases (e.g., COVID-19 and/or HIV) within minutes, allowing medical staff to diagnose and manage the patient's care more quickly. Where symptoms are life-threatening, a saving of 10 or 15 minutes in the diagnostic workup could be critical.

### Certain Aspects and Embodiments

Certain aspects of the present disclosure relate to a method of detecting the presence or absence of an analyte in a sample, the method comprising: contacting the sample with a first binding molecule bound to a detectable label and incubating the sample with the first binding molecule; contacting the sample with a second binding molecule bound to a magnetic particle and incubating the sample with the second binding molecule; adding the sample to a capillary vessel comprising an elongated capillary body having a first end and a second end disposed distal the first end, a sample entry port proximal the first end of the capillary body, and a sample exit port distal the first end of the capillary body and proximal the second end of the capillary body; and assessing the presence of a signal from the detectable label within the capillary vessel; wherein: a magnetic field is applied to at least a portion of the capillary vessel; the presence of a signal within the capillary vessel is indicative of association of the analyte derived from the sample with the first binding molecule and the second binding molecule; and incubating the first binding molecule and the second binding molecule with the sample is sufficient to bind the first binding molecule to a first binding site on the analyte and the second binding molecule to a second binding site on the analyte.

In some embodiments, the method described herein is capable of detecting the presence of an analyte. In some embodiments, the presence of an analyte is determined by the presence of a positive signal detected by the method. In some embodiments, the method described herein is capable of detecting the absence of an analyte. In some embodiments, the absence of an analyte is determined by the absence of a positive signal (a negative signal) detected by the method.

Certain aspects of the present disclosure relate to a diagnostic instrument adapted to receive a capillary vessel comprising an elongated capillary body having a first end and a second end disposed distal the first end, a sample entry port proximal the first end of the capillary body, and a sample exit port distal the first end of the capillary body and proximal the second end of the capillary body; wherein the diagnostic instrument further comprises: means for application of a magnetic field to a portion of the capillary body between the sample entry port and the sample exit port; and means for detection of a signal in a portion of the capillary body between the sample entry port and the sample exit port.

Certain aspects of the present disclosure relate to a diagnostic instrument, comprising: a capillary vessel comprising an elongated capillary body having a first end and a second end disposed distal the first end, a sample entry port proximal the first end of the capillary body, and a sample exit port distal the first end of the capillary body and proximal the second end of the capillary body; means for application of a magnetic field to a portion of the capillary body between the sample entry port and the sample exit port; and means for detection of a signal in a portion of the capillary body between the sample entry port and the sample exit port.

In some embodiments, the capillary vessel is a three-dimensional (3D)-printed capillary vessel or MiCRIA assay assembly. In some embodiments, the capillary vessel is a manufactured capillary vessel or MiCRIA assay assembly. In some embodiments, the capillary vessel is an assembled capillary vessel or MiCRIA assay assembly. In some embodiments, the capillary vessel or MiCRIA assay assembly is molded. In some embodiments, the capillary vessel or MiCRIA assay assembly is injection molded. In some embodiments, the capillary vessel comprises a U-shaped holder, a central portion, a septum, and a reservoir chamber. In some embodiments, the septum is rubber. In some embodiments, the U-shaped holder is affixed to the central portion by pins. In some embodiments, the pins are plastic. In some embodiments the reservoir chamber is affixed to the central portion by fastening means. In some embodiments, the fastening means are screws or tacks. In some embodiments, the screws are metal. In some embodiments, the septum is compressed between the central portion and the reservoir chamber by the fastening means and held in place. In some embodiments, a capillary body is inserted into the central portion through a hole in the central portion. In some embodiments, a filter is coupled to the capillary body and placed in the center of the U-shaped holder. In some embodiments, the filter is a cigarette filter. In some embodiments, a sample is placed in the reservoir chamber. In some embodiments, the sample flows from the reservoir chamber to the filter placed in the U-shaped holder by capillary action. In some embodiments, the capillary vessel further comprises means for application of a magnetic field. In some embodiments, the capillary vessel further comprises an excitation source. In some embodiments, the reservoir chamber is detachable from the central portion. In some embodiments, the reservoir chamber is attached to the central portion by fastening means. In some embodiments, a septum is compressed between the reservoir chamber and the central portion when the MiCRIA assay instrument is assembled. In some embodiments, the septum comprises an opening. In some embodiments, the opening is adapted to receive a capillary body. In some embodiments, the opening is oblong. In some embodiments, a capillary body is inserted through the opening of the septum and into the central portion. In some embodiments, inserting the capillary body into the central portion through the opening of the septum creates a liquid-proof seal. In some embodiments, inserting the capillary body into the central portion through the opening of the septum creates an air-tight seal. In some embodiments, the capillary body is inserted through the septum. In some embodiments, the capillary body is coupled to the septum. In some embodiments, the coupling creates a liquid-proof seal. In some embodiments, the coupling creates an air-tight seal. In some embodiments, the U-shaped holder is attached to the central portion by pins. In some embodiments, the pins are plastic. In some embodiments, the use of pins allows the U-shaped holder to be detachable from the central portion. In some embodiments, the U-shaped holder holds a filter. In some embodiments, the filter is a cigarette filter. In some embodiments, the use of pins allows the U-shaped holder to be detachable from the central portion, which allows the filter to be replaced.

In some embodiments, the manufactured capillary vessel is a part of an MiCRIA assay assembly. In some embodiments, the MiCRIA assay assembly comprises: a U-shaped holder, a central portion, a septum, and a reservoir chamber. In some embodiments, the septum is rubber. In some embodiments, the U-shaped holder is attached to the central portion by one or more pins. In some embodiments, the pins are plastic. In some embodiments, the reservoir chamber is affixed to the central portion by fastening means. In some embodiments, the fastening means are screws or tacks. In some embodiments, the screws are metal. In some embodiments, each individual part is manufactured. In some embodiments, each individual part is manufactured using a mold. In some embodiments, each individual part is injection molded. In some embodiments, the MiCRIA assay assembly is assembled prior to being received by the end user. In some embodiments, the MiCRIA assay assembly is manufactured as a single piece. In some embodiments, an end user obtains the MiCRIA assay assembly, the filter, the capillary body, and reagents separately. In some embodiments, an end user obtains the MiCRIA assay assembly and a kit comprising the filter, the capillary body, and the reagents. In some embodiments, the MiCRIA assay assembly is a single-use device.

Certain aspects of the present disclosure relate to a MiCRIA housing assembly. In some embodiments, the MiCRIA housing assembly is designed to house a MiCRIA assay assembly described herein. In some embodiments, the MICRIA housing assembly comprises a top portion and a bottom portion. In some embodiments, the bottom portion is detachable from the top portion. In some embodiments, the bottom portion is adapted to receive means for application of a magnetic field as described herein. In some embodiments, the magnet is affixed to the bottom portion of the MiCRIA housing assembly. In some embodiments, the magnet is non-permanently attached to the bottom portion of the MiCRIA housing assembly. In some embodiments, the bottom portion is adapted to receive the MiCRIA assay assembly. In some embodiments, the MiCRIA assay assembly is affixed to the bottom portion of the MiCRIA housing assembly. In some embodiments, the MiCRIA assay assembly is non-permanently attached to the bottom portion of the MiCRIA housing assembly. In some embodiments, the assay assembly is a part of the bottom portion of the MiCRIA housing assembly. In some embodiments, the non-permanent attachment comprises fastening means, Velcro, clips, or by tension contact with a section of the bottom portion adapted to receive the MiCRIA assay assembly. In some embodiments, the affixation is adhesive, nails, screws, staples, tacks, bolts, or rivets. In certain embodiments, the adhesive is glue. In certain embodiments, the glue is hot glue or superglue. In certain embodiments, the adhesive is epoxy. In certain embodiments, the affixing means comprises compression.

In some embodiments, the bottom portion of the MiCRIA housing assembly is adapted to receive the top portion of the MiCRIA housing assembly. In some embodiments, the top portion of the MiCRIA housing assembly attaches the bottom portion of the MiCRIA housing assembly by means of Velcro, clips, tension contact, or compression. In some embodiments, the top portion of the MiCRIA housing assembly attaches to the bottom portion of the MiCRIA housing assembly by gravity. In some embodiments, the top portion of the MiCRIA housing assembly forms a light-tight seal with the bottom portion of the MiCRIA housing assembly. In some embodiments, the top portion of the MICRIA housing assembly forms a substantially light-tight seal with the bottom portion of the MICRIA housing assembly. In some embodiments, the top portion of the MiCRIA housing assembly is further separated into two parts. In some embodiments, the two parts of the top portion of the MiCRIA housing assembly are attached by means of Velcro, clips, tension contact, or compression.

In some embodiments, the top portion of the MiCRIA housing assembly is adapted to receive one or more cameras. In some embodiments, the top portion of the MiCRIA housing assembly further comprises one or more slots adapted to receive the one or more cameras. In some embodiments, the one or more cameras attach to the top portion of the MiCRIA housing assembly by means of fastening means, Velcro, clips, tension contact, or compression. In some embodiments, the one or more cameras is non-permanently attached to the top portion of the MiCRIA housing assembly. In some embodiments, the one or more cameras are coupled to the top portion of the MiCRIA housing assembly such that the one or more cameras are directed toward the bottom portion of the MiCRIA housing assembly adapted to receive the MiCRIA assay assembly. In some embodiments, the one or more camera are digital cameras. In some embodiments, the MiCRIA housing assembly comprises one or more cameras. In some embodiments, the MiCRIA housing assembly comprises two cameras. In some embodiments, one or more cameras comprise one or more photodetectors. In some embodiments, one or more cameras comprise two photodetectors that can be toggled. In some embodiments, the two cameras comprising the same photodetector are utilized. In some embodiments, the two cameras comprise different photodetectors. In some embodiments, the photodetectors are point-based. In some embodiments, the photodetectors are camera-based. In some embodiments, the one or more cameras are charge-coupled devices (CCD). In some embodiments, the one or more cameras are active-pixel sensors (CMOS). In some embodiments, the MiCRIA housing assembly comprises two or more cameras. In some embodiments, the two or more cameras are directed to approximately the same point. In some embodiments, the two or more cameras are directed to different points. In some embodiments, the MiCRIA assay assembly further comprises diffraction grading to spread wavelengths into different physical locations in the MiCRIA housing assembly. In some embodiments, the MiCRIA housing assembly is adapted to receive a filter. In some embodiments, the filter is a filter disk. In some embodiments, the filter is adapted to filter any light derived from a source other than the MiCRIA assay assembly. In some embodiments, the filter is adapted to filter wavelengths emitted from any source other than the MiCRIA assay assembly (e.g., ambient light). In some embodiments, only light derived from the MiCRIA assay assembly is able to pass through the filter. In some embodiments, only wavelengths emitted from the MiCRIA assay assembly are able to pass through the filter. In some embodiments, the one or more cameras in the MiCRIA housing assembly only detect light that passes through the filter. In some embodiments, the one or more cameras in the MiCRIA housing assembly only detect wavelengths that pass through the filter. In some embodiments, only wavelengths of about 610 nm pass through the filter. In some embodiments, the filter has a narrow passthrough range. In some embodiments, the filter is a bandpass filter. In some embodiments, the filter is a longpass filter. In some embodiments, only wavelengths greater than about 500 nm, greater than about 510 nm, greater than about 520 nm, greater than about 530 nm, greater than about 540 nm, greater than about 550 nm, greater than about 560 nm, greater than about 570 nm, greater than about 580 nm, greater than about 590 nm, greater than about 600 nm, greater than about 610 nm pass through the filter. In some embodiments, only wavelengths greater than about 550 nm pass through the filter.

In some embodiments, the top portion of the MiCRIA housing assembly is adapted to receive an excitation source as described herein. In some embodiments, the top portion of the MiCRIA housing assembly further comprises one or more slots adapted to receive the one or more excitation sources. In some embodiments, the one or more excitation sources are affixed to the top portion of the MiCRIA housing assembly. In some embodiments, the one or more excitation sources excite the same wavelength. In some embodiments, the one or more excitation sources excite different wavelengths.

In some embodiments, the MiCRIA housing assembly is battery-powered. In some embodiments, the MiCRIA housing assembly is powered by an external energy source. In some embodiments, the reservoir chamber of the MiCRIA assay assembly is accessible from outside the MiCRIA housing assembly. In some embodiments, the addition of liquid to the MiCRIA housing assembly operably engages the MiCRIA housing assembly of the MiCRIA housing assembly. In some embodiments, placing the top portion of the MiCRIA housing assembly onto the bottom portion of the MiCRIA housing assembly operably engages the MiCRIA housing assembly. In some embodiments, the MiCRIA housing assembly further comprises an external switch to operably engage the MiCRIA housing assembly. In some embodiments, operably engaging the MiCRIA housing assembly initiates one or more of power supply to the excitation source(s) and/or camera(s), data recording, initiation of flow from the reservoir chamber into the capillary body, and timing of the MiCRIA assay.

Certain aspects of the present disclosure relate to a kit, comprising: a first reagent comprising a first binding molecule bound to a detectable label; and a second reagent comprising a second binding molecule bound to a magnetic particle.

Certain aspects of the present disclosure relate to a kit, comprising: a first reagent comprising a first binding molecule bound to a detectable label; a second reagent comprising a second binding molecule bound to a magnetic particle; and a capillary pad.

Certain aspects of the present disclosure relate to a kit, comprising: a first reagent comprising a first binding molecule bound to a detectable label; a second reagent comprising a second binding molecule bound to a magnetic particle; and a capillary vessel comprising an elongated capillary body having a first end and a second end disposed distal the first end, a sample entry port proximal the first end of the capillary body, and a sample exit port distal the first end of the capillary body and proximal the second end of the capillary body.

Certain aspects of the present disclosure relate to a kit, comprising: a first reagent comprising a detectable label adapted to be bound to a first binding molecule; a second reagent comprising a magnetic particle adapted to be bound to a second binding molecule; a capillary vessel comprising an elongated capillary body having a first end and a second end disposed distal the first end, a sample entry port proximal the first end of the capillary body, and a sample exit port distal the first end of the capillary body and proximal the second end of the capillary body.

Certain aspects of the present disclosure relate to a kit, comprising a capillary vessel comprising an elongated capillary body having a first end and a second end disposed distal the first end, a sample entry port proximal the first end of the capillary body, and a sample exit port distal the first end of the capillary body and proximal the second end of the capillary body; wherein: the capillary vessel is adapted to interface with a diagnostic instrument comprising: means for application of a magnetic field to a portion of the capillary body between the sample entry port and the sample exit port; and means for detection of a signal in a portion of the capillary body between the sample entry port and the sample exit port.

In some embodiments, the capillary is a separate consumable that is inserted into a reader assembly comprising a magnet, excitation source, and optical reader. In some embodiments, the reader assembly is the primary instrument. In some embodiments, a generic capillary vessel is inserted into the reader assembly. In some embodiments, the reader assembly is adapted to receive a capillary vessel and the capillary vessel inserted into the reader assembly is adapted to receive any form of analyte. In some embodiments, reagents for different types of analytes are separate consumable.

In some embodiments, the analyte or target molecule is a protein. In some embodiments, the analyte is a fragment of a protein. In some embodiments, the analyte is a peptide. In some embodiments, the analyte is an antibody. In some embodiments, the analyte is an antigen. In some embodiments, the antigen is a VlsE protein or peptide. In some embodiments, the antigen is a recombinant VlsE protein or peptide. In some embodiments, the antigen is VlsE crosslinked to carboxylated particles. In some embodiments, the antigen is a C6 peptide. In some embodiments, the antigen is an HIV IDR-m peptide. In some embodiments, the antigen is a recombinant gp41. In some embodiments, the analyte or target molecule is bound to streptavidin. In some embodiments, the analyte or target molecule is biotinylated. In some embodiments, the magnetic particle is bound to streptavidin. In some embodiments, the magnetic particle is biotinylated. In some embodiments, the detectable label is bound to streptavidin. In some embodiments, the detectable label is biotinylated. In some embodiments, the streptavidin is conjugated to biotin. In some embodiments, the analyte or target molecule is conjugated to the magnetic particle via a biotin-streptavidin link. In some embodiments the analyte or target molecule is conjugated to the detectable label via a biotin-streptavidin link. In some embodiments, the analyte is a nucleic acid. In some embodiments, the analyte is an oligonucleotide. In some embodiments, the analyte is a longer DNA or RNA sequence. In some embodiments, the oligonucleotide is naturally-occurring. In some embodiments, the oligonucleotide is synthesized or amplified by enzymatic means. In some embodiments, the enzymatic means is PCR. In some embodiments, the oligonucleotide is a polymerase chain reaction product. In some embodiments, the analyte is a glycan or other carbohydrate molecule. In some embodiments, the analyte is a lipid. In some embodiments, the analyte is a polymer. In some embodiments, the analyte is a combination of protein, carbohydrate, and/or lipid components. In some embodiments, the analyte is a small molecule. In some embodiments, the analyte is a drug or a vitamin. In some embodiments, the analyte is a hormone or other natural compound. In some embodiments, detection of an antibody is indicative of a current or recent infection. In some embodiments, the antibody is produced in response to an infection. In some embodiments, the analyte is an antigen. In some embodiments, detection of an antigen is indicative of a current or recent infection. In some embodiments, wherein the analyte is a protein, a peptide, a polypeptide, or an oligonucleotide. In some embodiments, the sample is from a subject who has or is suspected of having an infectious disease. In some embodiments, the sample is from a subject who has or who is suspected of having a non-infectious disease. In some embodiments, the sample is from a subject who has or is suspected of having cancer. In some embodiments, the sample is from a subject who has or is suspected of having a traumatic brain injury. In some embodiments, the first binding molecule and the second binding molecule are the same. In some embodiments, the first binding molecule and the second binding molecule are different. In some embodiments, the target molecule is an IgG antibody, and crosslinking is effected by coating the paramagnetic particle with Protein A/G to bind the Fc portion of the antibody molecule, or by coating the paramagnetic particle with the specific antigen recognized by the antibody. In some embodiments, the reporter is a fluorescent or visibly labeled particle or probe conjugated with the specific antigen. In some embodiments, fluorescence provides an advantageous contrast, as the signal is read against a background of magnetic particles. In one embodiment, the reporter consists of fluorescent Europium chelate particles that are readily coated with antigens. Europium exhibits intense fluorescence at 615 nm with excitation at 365 nm, offering higher sensitivity of detection than certain visibly dyed particles (Juntunen et al., Anal Biochem 2012 September; 428(1):31-8; Luo et al. J Dairy Sci 2017; 100(7):5176-87).

In some embodiments, the first binding molecule is a protein. In some embodiments, the first binding molecule is an antigen, an antibody, or a surface protein.

In some embodiments, the second binding molecule is a protein. In some embodiments, the second binding molecule is an antigen, an antibody, or a surface protein.

In some embodiments, the first binding molecule is a protein and the second binding molecule is a protein. In some embodiments, the first binding molecule is a protein and the second binding molecule is an antibody. In some embodiments, the first binding molecule is a protein and the second binding molecule is an antigen. In some embodiments, the first binding molecule is a protein and the second binding molecule is a surface protein. In some embodiments, the first binding molecule is an antibody and the second binding molecule is a protein. In some embodiments, the first binding molecule is an antibody and the second binding molecule is an antibody. In some embodiments, the first binding molecule is an antibody and the second binding molecule is an antigen. In some embodiments, the first binding molecule is an antibody and the second binding molecule is a surface protein. In some embodiments, the first binding molecule is an antigen and the second binding molecule is a protein. In some embodiments, the first binding molecule is an antigen and the second binding molecule is an antibody. In some embodiments, the first binding molecule is an antigen and the second binding molecule is an antigen. In some embodiments, the first binding molecule is an antigen and the second binding molecule is a surface protein. In some embodiments, the first binding molecule is a surface protein and the second binding molecule is a protein. In some embodiments, the first binding molecule is a surface protein and the second binding molecule is an antibody. In some embodiments, the first binding molecule is a surface protein and the second binding molecule is an antigen. In some embodiments, the first binding molecule is a surface protein and the second binding molecule is a surface protein.

In some embodiments, the first binding site is an antigen-binding site, an antibody-binding site, or an antibody Fc region. In some embodiments, the second binding site is an antigen-binding site, an antibody-binding site, or an antibody Fc region. In some embodiments, the method further comprises adding a wash buffer to the capillary vessel after the sample. In some embodiments, the sample is added to a wash buffer prior to being added to the capillary vessel. In some embodiments, the steps of the method are completed in order, simultaneously, or in any order. In some embodiments, the kit further comprises a third reagent comprising a wash buffer. In some embodiments, the MiCRIA assays described herein are adapted for detection of nucleic acids. In some embodiments, the magnetic particle and the labeled particle can each be conjugated to a nucleic acid sequence (oligonucleotide) enabling the capture of homologous DNA or RNA from a sample.

As will be appreciated by a skilled artisan, the method described herein is subject to many other variations. In some embodiments, the presence of an analyte is determined by the presence of a signal from a detectable label. In some embodiments, the detectable label is luminescent. In some embodiments, the detectable label is fluorescent. In some embodiments, the detectable label is a Europium label. In some embodiments, the Europium label is Europium chelate latex particles. In some embodiments, the Europium particles have a diameter ranging from 0.1-0.3 μm In some embodiments, the Europium particles have a diameter of 0.1 μm. In some embodiments, the Europium particles have a diameter of 0.2 μm. In some embodiments, the Europium particles have a diameter of 0.3 μm. In some embodiments, the detectable label is a dye. In some embodiments, the detectable label is an enzyme label are used, and the associated substrate is introduced to the capillary. In some embodiments, the detectable label is fluorescent probe other than Europium. In some embodiments, the detectable label is a probe that is provided in molecular form rather than chelated within particles as is Europium. In some embodiments, the detectable label is a phycoerythrin, a brilliant violet label, a marina blue label, an Alexa Fluor label, fluorescein isothiocyanate, rhodamine, a cyanine, BODIPY, gold nanoclusters, quantum dots, carbon dots, or polymer dots. In some embodiments, the detectable label is fluorescent quantum dots (Deerinck, Toxicol Pathol. 2008, 36(1):112-16). In some embodiments, the detectable label is polymer dots (Kauser, et al. Journal of Plastic Film & Sheeting. 2021; 37(4):510-28). In some embodiments, the detectable label is a visible dyed particle. In some embodiments, the detectable label is a magnetic label-bearing particle. In some embodiments, the detectable label is a non-magnetic label-bearing particles. In some embodiments, the detectable label is Janus particles.

In some embodiments, the detectable label is Europium. In some embodiments, the detectable label is a fluorescent label, a luminescent label, a colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, a fluorescent label emits light energy only when excited. In some embodiments, a luminescent label emits light energy during excitation and for some time after excitation ceases. In some embodiments, Europium is fluorescent. In some embodiments, Europium is luminescent. In some embodiments, Europium is fluorescent and luminescent. In some embodiments, the assay described herein uses a luminescent Europium. In some embodiments, the assay described here uses a fluorescent Europium. In some embodiments, the assay described herein uses a Europium that is fluorescent and luminescent. In some embodiments, the colorimetric label is a horseradish peroxidase label or alkaline phosphatase. In some embodiments, the detectable label is adapted to be detected by a substrate. In some embodiments, the substrate is selected from the group consisting of: ABTS, OPD, AmplexRed, DAB, AEC, TMB, Homovanillic Acid, and Luminol. In some embodiments, the substrate is 3,3′,5,5′-tetramethylbenzidine (TMB). In some embodiments, the substrate is Luminol.

In some embodiments, an internal control is incorporated through use of optical reporter particles with a different antigen and different emission wavelength, which are separately detectable via use of a corresponding excitation source. In some embodiments, the internal control is Super Bright 436 dye (with emission at 436 nm). In some embodiments, the internal control is Qdot 525 (with emission at 525 nm). In some embodiments, the internal control is marina blue. Marina blue is a fluorescent dye with a 364 nm excitation peak and a 461 nm emission peak. In some embodiments, the internal control is Brilliant Violet. Brilliant Violet is a tandem dye with a 405 nm excitation peak and a 711 nm emission peak. In some embodiments, the internal control particles are conjugated to an anti-IgG antibody. In some embodiments, internal control particles conjugated to an anti-IgG antibody are useful for testing blood samples. In some embodiments, the internal control is a magnetic particle conjugated to an antibody that binds an antigen not found in nature. In some embodiments, the antibody is a synthetic peptide or other synthesized small molecule that is conjugated to the labelled detection reagent. Certain such embodiments provide a more universal internal control for any sample type. In some embodiments, this approach provides multiplexing capability. In some embodiments, multiplexing includes conjugating more than one detectable label or magnetic particle with more than one binding molecule. In certain such embodiments, a reader is used that has the capability to separately detect signals from each label. In some embodiments, the labels are fluorescent labels with distinct emission wavelengths that do not overlap. Certain such multiplexing embodiments allow for the testing of more than one antibody, antigen or other analyte in a single assay. In some embodiments, multiplexing is used in a diagnostic setting. In some embodiments, the diagnostic setting is a patient with an unidentified respiratory infection. In some embodiments, the respiratory infection is COVID-19, influenza, RSV, or another virus.

In some embodiments, the target molecule is a nucleic acid sequence, e.g. an amplicon resulting from PCR or other amplification, and the paramagnetic particle contains an immobilized sequence complementary to one end of the amplicon, and the reporter particle is coated with a second sequence complementary to the opposite end. In some embodiments, the coating of the reporter particle is facilitated by adding poly (dA) or a specific capture sequence to the PCR primer. Analogous hybridization approaches involving two different probes have been used in a variety of nucleic acid detection assays (Morrissey et al., Anal Biochem. 1989 Sep. 1; 181(2):345-59; Bach et al., J Microbiol Methods. 1999 August; 37(2):187-92. Chandler et al., J. Clin. Micro. 1993; 31(10)).

In some embodiments, the magnetic particle is a magnetic bead. In some embodiments, the magnetic particle is a magnetic nano-particle. In some embodiments, the magnetic particle is a magnetic micro-particle. In some embodiments, the magnetic particle is a paramagnetic particle. A paramagnetic particle, as described herein, refers to a particle that only exhibits magnetic properties in the presence of a magnetic field.

In some embodiments, the magnetic particle and the labeled particle can each be coated or conjugated with an antibody for use in an antigen capture assay; non-competing antibodies can be used on the two particles to capture an antigen with non-repeating epitopes.

In some embodiments, the magnetic field is generated by a magnet. In some embodiments, the magnet is a rare earth magnet. In some embodiments, the rare earth magnet comprises neodymium. In some embodiments, the rare earth magnet is a samarium-cobalt (SmCo) magnet. In some embodiments, the magnet is paramagnetic. In some embodiments, the magnet is an electromagnet. In some embodiments, the magnet is a composition comprising a mixture of aluminum, nickel, cobalt, and iron. In some embodiments, the magnet is ceramic. In some embodiments, the magnet is ferrite. In some embodiments, the magnet is a small neodymium magnet. In some embodiments, the magnet is approximately 2 mm in the longest dimension.

In some embodiments, the magnetic field is generated by magnetic field generation means. In some embodiments, the magnetic field generation means comprises a magnet. In some embodiments, the magnetic field generation means is an electromagnetic device. In some embodiments, the magnetic field separates analyte bound to the first binding molecule and the second binding molecule from analyte not bound to the first binding molecule and the second binding molecule.

In some embodiments, the capillary vessel is a tube. In some embodiments, the capillary vessel is substantially cylindrical. In some embodiments, the capillary vessel is a cuboid. In some embodiments, the capillary vessel comprises one or more substantially planar surfaces along its longitudinal dimension. In some embodiments, the capillary vessel is in fluid communication with a capillary pad.

In some embodiments the entry port can be cylindrical, conical, or other shape capable of holding a greater volume (e.g. 250-500 microliters) of solution than the capillary volume itself (about 50 microliters in the examples used for most experiments). In some embodiments, the wicking filter can be of any shape and material that can absorb liquid. In some embodiments, cellulose cigarette filters have been shown to function as wicking filters, and can be incorporated into the capillary device itself as a single consumable unit, or attached separately when the assay is carried out.

In some embodiments, the kit further comprises a capillary vessel.

In some embodiments, the kit further comprises a capillary pad.

In some embodiments, the kit further comprises instructions for use.

In some embodiments, no reagent is bound to the capillary vessel.

In some embodiments, the capillary vessel is coupled with means to induce the sample from the sample entry port towards the sample exit port (“inducing means”). In some embodiments, the inducing means is passive. In some embodiments, the passive inducing means do not require pumping. In some embodiments, the passive inducing means is capillary force or action that draws the test solution through the capillary vessel. In some embodiments, the passive inducing means is aspiration by capillary action. In some embodiments, inducing means comprise means to remove liquid contents from the exit port of the capillary vessel. In some embodiments, the means to remove liquid contents is passive. In some embodiments, the passive means to remove liquid contents is a capillary pad or wicking filter. In some embodiments, the means to remove liquid contents is an active pump. In some embodiments, the inducing means is an active pump. In some embodiments, the active pump is a peristaltic pump. In some embodiments, the inducing means is a vacuum. In some embodiments, the inducing means is airflow. In some embodiments, pumping is not used to induce the sample from the sample entry port towards the sample exit port.

In some embodiments, the inducing means is pumping means. The term “pumping means,” as used herein, refers to any active force that moves the sample from the entry port to the exit port. In some embodiments, pumping means includes an active pump, a peristaltic pump, or another means of pumping, as will be understood by a skilled artisan.

In some embodiments, the detection step is accomplished passively by optical detection of fluorescence associated with beads captured by a magnet that is a fixed part of the assembly.

In some embodiments, the inducing means remove the liquid contents from the capillary. In some embodiments, the inducing means is a capillary pad. In some embodiments, the capillary pad is a substantially cylindrical filter. In come embodiments, the substantially cylindrical filter is a cigarette filter ([d] in FIG. 2). In some embodiments, the inducing means comprises a connection via tubing. In some embodiments, the inducing means is a pump. In some embodiments, the pump is a peristaltic pump.

In some embodiments, a volume of additional wash buffer is added to the end of the capillary via the reservoir ([b] in FIG. 2) to flush away any residual unbound fluorescent label. In some embodiments, the wash buffer is between about 50 and about 500 μL. In some embodiments, the wash buffer is between about 100 and about 400 μL. In some embodiments, the wash buffer is between about 200 and about 300 μL. In some embodiments, the wash buffer is about 250 microliters. In some embodiments, the wash buffer flows through the capillary by way of the inducing means. In some embodiments, the wash buffer is absorbed by the capillary pad. In some embodiments, the wash buffer is removed by a pump.

In some embodiments, the fluorescent label bound to the antibody or analyte that is in turn bound to the magnetic particles is detected in real time as liquid flows through the capillary by an optical detection device. In some embodiments, the optical detection device is a camera or photodiode.

In some embodiments, the total turnaround time from start to finish is less than 5 minutes. In some embodiments, the entire assay is completed in less than 10 minutes. In some embodiments, the incubation of the sample and binding molecules is approximately 1 minute. In some embodiments, the assay is completed in as little as 1-2 minutes.

In some embodiments, the diagnostic instrument further comprises an excitation source adapted to direct energy to a portion of the capillary body between the sample entry port and the sample exit port. In some embodiments, an excitation source directs energy to a portion of the capillary body. In some embodiments, the excitation source is adapted to direct energy to an area at or near the middle of the capillary body. In some embodiments, the excitation source is an electron excitation source.

In some embodiments, the detectable label is detected within 100 seconds after the sample is added to the capillary vessel. In some embodiments, the detectable label is detected approximately 10 seconds, approximately 20 seconds, approximately 30 seconds, approximately 40 seconds, approximately 50 seconds, approximately 60 seconds, approximately 70 seconds, approximately 80 seconds, approximately 90 seconds, or approximately 100 seconds after the sample is added to the capillary vessel. In some embodiments, the detectable label is detected approximately 60 seconds after the sample is added to the capillary vessel. In some embodiments, the detectable label is detected between approximately 10 and approximately 110 seconds, between approximately 20 and approximately 100 seconds, between approximately 30 and approximately 90 seconds, between approximately 40 and approximately 80 seconds, or between approximately 50 and approximately 70 seconds after the sample is added to the capillary vessel.

In some embodiments, the diagnostic instrument further comprises a capillary pad adapted to be in fluid communication with the sample exit port. In some embodiments, the method or diagnostic instrument further comprises means for inducing a sample from the sample entry port to the sample exit port. In some embodiments, the magnetic field is concentrated on a portion of the capillary body in or around the middle of the capillary vessel. In some embodiments, the magnet is positioned roughly at the center of the capillary vessel.

In some embodiments, the signal is detected within the portion of the capillary body where the magnetic field is applied. In some embodiments, analytes bound to the first binding molecule and the second binding molecule are captured at the site of the magnetic field. In some embodiments, incubating is carried out at ambient temperature. In some embodiments, incubating is carried out at 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. In some embodiments, incubating is carried out at 37° C. In some embodiments, incubating is carried out at 40° C. In some embodiments, incubating is carried out for less than 1 minute, 1 minute, 2 minutes, 3 minutes, 4 minutes, or 5 minutes. In some embodiments, incubating is carried out for less than 1 minute. In some embodiments, incubating is carried out for approximately 1 minute. In some embodiments, incubating is carried out for approximately 2 minutes. In some embodiments, incubating is carried out for approximately 3 minutes. In some embodiments, incubating is carried out for between approximately 1 and approximately 5 minutes, between approximately 2 and approximately 4 minutes, between approximately 2 and approximately 3 minutes, or between approximately 3 and approximately 4 minutes.

In some embodiments, the capillary vessel is enclosed in a substantially light-tight enclosure. The term “substantially light-tight enclosure,” as used herein, refers to an enclosure that significantly limits the amount of light that is able to enter the enclosure. While it may not be possible to completely eliminate light entry, a substantially light-tight enclosure will eliminate entry of most light. In some embodiments, the sample is not exposed to light once it is added to the capillary vessel. In some embodiments the method, diagnostic instrument, or kit further comprises a displaceable barrier in contact with the exit port. In some embodiments, adding the sample comprises adding the sample to the sample entry point. In some embodiments, adding the sample comprises adding the sample to a reservoir fluidically coupled to the sample entry port. In some embodiments, capillary action does not occur immediately after the sample is added to the sample entry port.

In some embodiments, the method further comprises a displaceable barrier between the sample entry port and the capillary vessel. In some embodiments, the method further comprises a displaceable barrier within the capillary vessel. In some embodiments, the method further comprises a displaceable barrier between the capillary vessel and the sample exit port. In some embodiments, the displaceable barrier prevents capillary action. In some embodiments, the displaceable barrier is displaced by a displacing member. In some embodiments, the displaceable barrier is displaced when the capillary vessel is inserted into a reading device adapted to receive the capillary vessel. In some embodiments, insertion of the capillary vessel into the reading device triggers a timer to trigger assessing the presence of the detectable label at a predetermined time after insertion. In some embodiments, the predetermined time is between approximately 10-110 seconds, approximately 20-100 seconds, approximately 30-90 seconds, approximately 40-80 seconds, or approximately 50-70 seconds after insertion. In some embodiments, the diagnostic instrument or kit further comprises a displaceable barrier between the sample entry port and the capillary vessel. In some embodiments, the diagnostic instrument or kit further comprises a displaceable barrier within the capillary vessel. In some embodiments, the diagnostic instrument or kit further comprises a displaceable barrier between the capillary vessel and the sample exit port. In some embodiments, the displaceable barrier is adapted to prevent capillary action. In some embodiments, the displaceable barrier is adapted to be displaced by a displacing member. In some embodiments, the displaceable barrier is adapted to be displaced upon insertion of the capillary vessel into a reading device adapted to receive the capillary vessel. In some embodiments, the reading device is adapted to trigger a timer to trigger assessment of the presence of the detectable label at a predetermined time after insertion. In some embodiments, the predetermined time is between approximately 10-110 seconds, approximately 20-100 seconds, approximately 30-90 seconds, approximately 40-80 seconds, or approximately 50-70 seconds after insertion. In some embodiments, the diagnostic instrument is adapted to open to receive the capillary vessel. The diagnostic instrument of any one of the preceding claims, wherein the diagnostic instrument is adapted to form a substantially light-tight interior space. In some embodiments, the diagnostic instrument further comprises a displacing member.

In some embodiments, the sample is a biological sample. In some embodiments, the biological sample is selected from the group consisting of: saliva, oral fluid, tears, urine, interstitial fluid, synovial fluid, nasal fluid, nasopharyngeal fluid, and cerebrospinal fluid. In some embodiments, the biological sample is blood. In some embodiments, the biological sample is blood, whole blood, dried blood, serum, plasma, or a blood fraction. In some embodiments, the sample is collected from a subject.

In some embodiments, the blood is collected from a subject suspected of having or who has an infectious disease. In some embodiments, the infectious disease is Lyme disease, human immunodeficiency virus, or SARS-CoV-2. In some embodiments, the blood is collected from a subject suspected of having or who has a non-infectious disease. In some embodiments, the blood is collected from a subject suspected of having or who has cancer. In some embodiments, the blood is collected from a subject suspected of having or who has a traumatic brain injury. In some embodiments, the subject is a human or a non-human animal. In some embodiments, the subject is human. In some embodiments, the sample is collected from a subject suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

One skilled in the art can, based on the above description, utilize the present invention to its fullest extent. The following specific embodiments are, therefore, merely illustrative, and are not limitative of the remainder of the disclosure in any way whatsoever. All publications cited in the present application are incorporated by reference for the purposes or subject matter referenced in this disclosure.

### EXAMPLES

### Example 1. Exemplary MiCRIA Assay Device

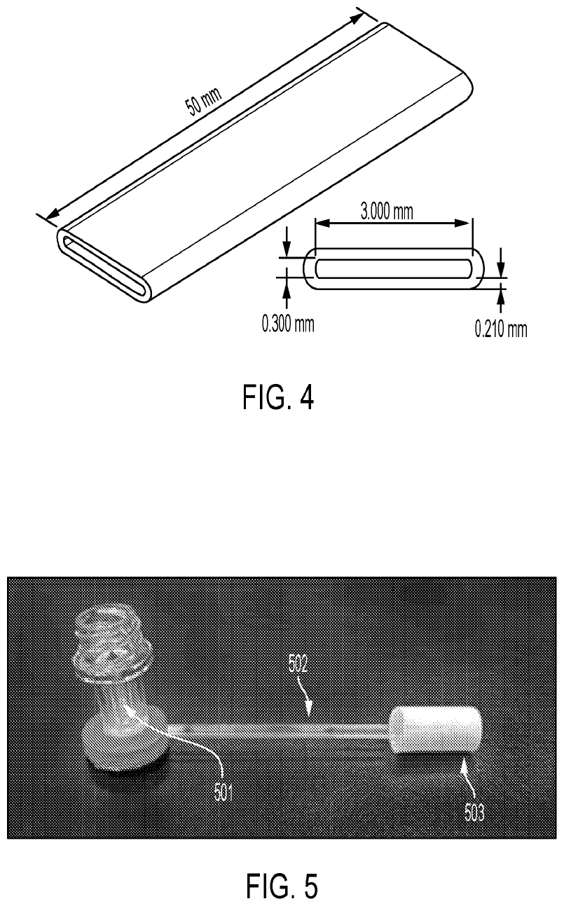

UV-transparent glass capillaries were obtained commercially from Vitrocom, with dimensions either 3 mm×0.3 mm (as shown in the schematic FIGS. 4) and 2 mm×0.1 mm, both 50 mm in length. As shown in FIG. 5, an entry port (501) was made consisting of a plastic sample cup with a hole drilled through the bottom (a reservoir), glued to a silicone rubber pad in which a short incision had been made, through which the end of the capillary vessel (502) was inserted to fluidically couple the entry port of the capillary vessel with the reservoir. The opposing end of the capillary vessel was placed in fluid communication with the capillary pad (a cigarette filter; 503).

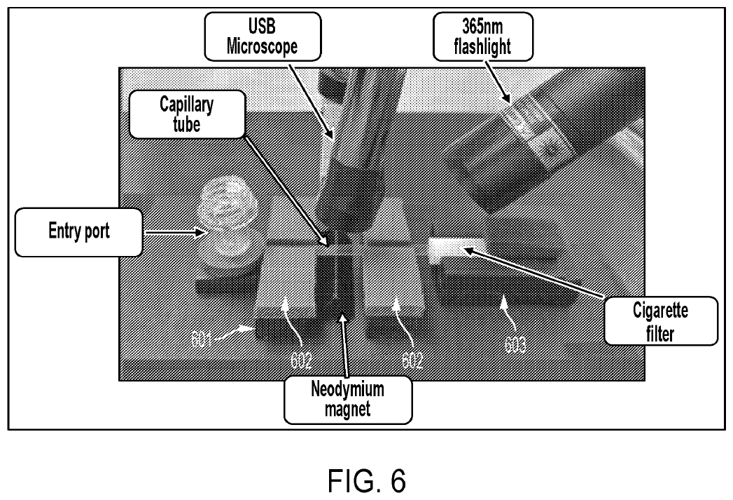

The assembly shown in FIG. 5 was included in the exemplary MiCRIA assay device shown in FIG. 6. A 32 mm×9 mm triangular magnet triangular neodymium magnet was positioned underneath a pair of support blocks (601) on top of which a pair of slotted rubber pads (602) held the capillary vessel in position by pressure. An area of the capillary vessel approximately aligned with the vertex of the magnet was illuminated by a focused 365 nm UV flashlight (Alonefire SV15, 3 watts). A USB microscope (Opti-Tekscope) with a CMOS sensor operating at 640×480 resolution was positioned over the magnet and connected to a laptop to visualize the detected signal using Debut, a JPEG video capture software package (NCH Software). During an assay run, a solution was introduced into reservoir, flowed down and into the capillary vessel via the entry port, flowed through the capillary vessel and was absorbed by the capillary pad. The capillary pad acted as a wicking pad, resulting in continuous flow until all liquid was absorbed. The capillary pad was held in place against the exit port of the capillary vessel by a press fit in a slotted mounting block (603).

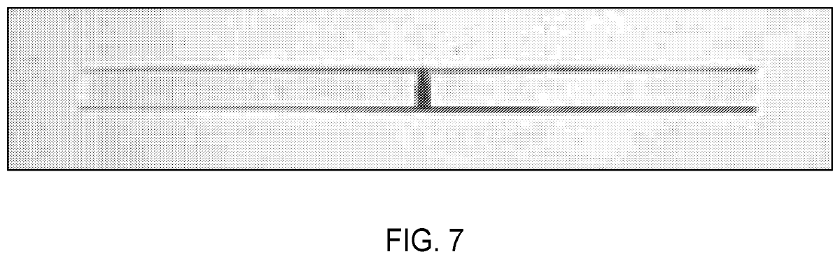

The intensity of the Europium signal was quantitated by importing JPEG images into ImageJ, a public domain, Java-based image processing program from the National Institutes of Health. The ability of the neodymium magnet to capture paramagnetic particles flowing rapidly through the capillary channel was confirmed. As shown in FIG. 7, an aliquot of 1 μm paramagnetic particles introduced into the entry port through the reservoir was immediately drawn into and through the channel by capillary action and adsorbed by the capillary pad, the entire process taking less than one minute and leaving the paramagnetic particles in a well-defined band directly over the magnet.

### Example 2: Capillary Assay Device Design

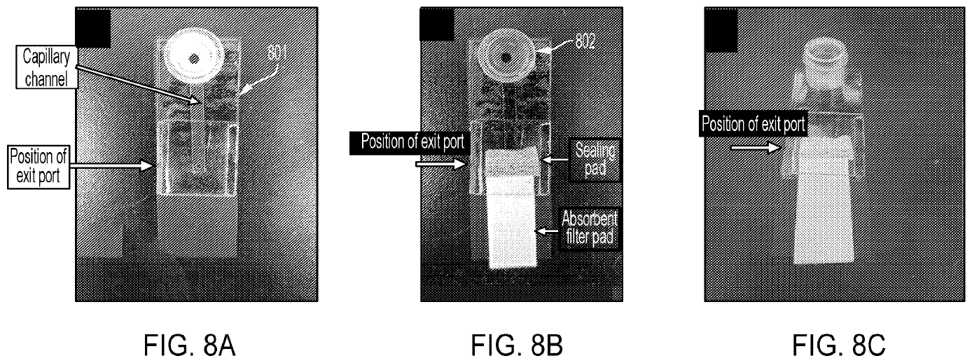

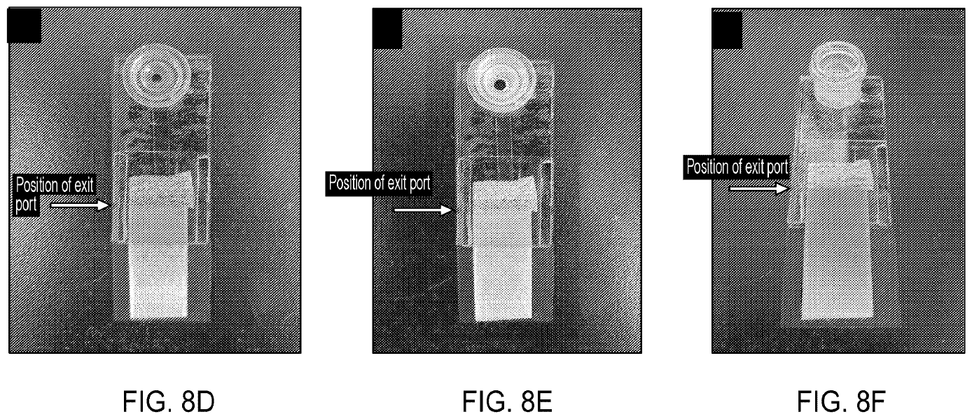

FIGS. 8A-8F demonstrate initiation of liquid flow through a capillary channel by horizontal displacement of a sealing pad and capillary pad over the capillary exit port. FIG. 8A shows an overhead view of a capillary assembly showing the capillary channel, reservoir over and fluidically coupled with the entry port, exit port at the distal end, and a sleeve (801) adapted to receive a capillary pad and sealing pad. FIG. 8B shows an overhead view of the capillary pad and sealing pad inserted in the sleeve. The exit port (indicated by a gray arrow) of the capillary vessel lay beneath and was engaged with the sealing pad. Colored dye was introduced into the reservoir (802) but did not flow into the capillary due to the engagement of the sealing pad with the exit port. FIG. 8C shows a perspective view of FIG. 8B. FIG. 8D shows an overhead view after the capillary pad and sealing pad were pushed several millimeters forward into the sleeve, such that the exit port lay beneath and was engaged with the capillary pad. Consequently, a portion of the colored dye began to be drawn into the capillary vessel, began to flow through the capillary channel and began to be absorbed by capillary pad. A portion of the colored dye remained in the reservoir. FIG. 8E shows an overhead view after the entirety of the colored dye flowed from the reservoir, through the entry port, into the capillary pad, which absorbed all of the colored dye introduced into the entry port. FIG. 8F shows a perspective view of FIG. 8E.

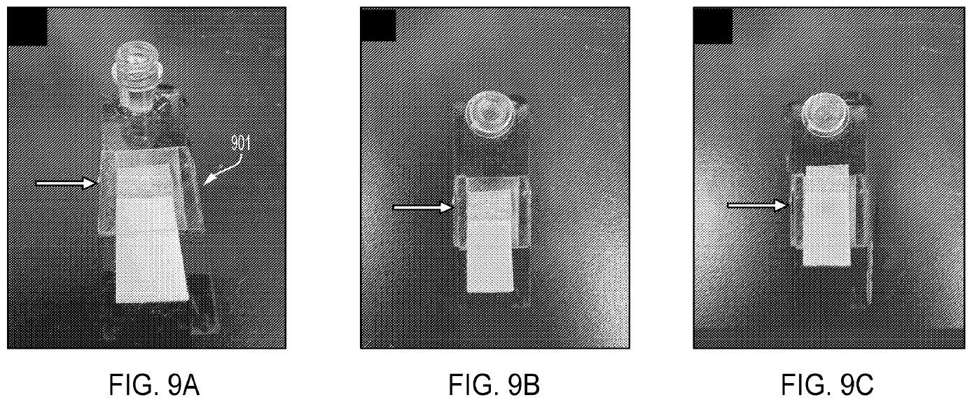

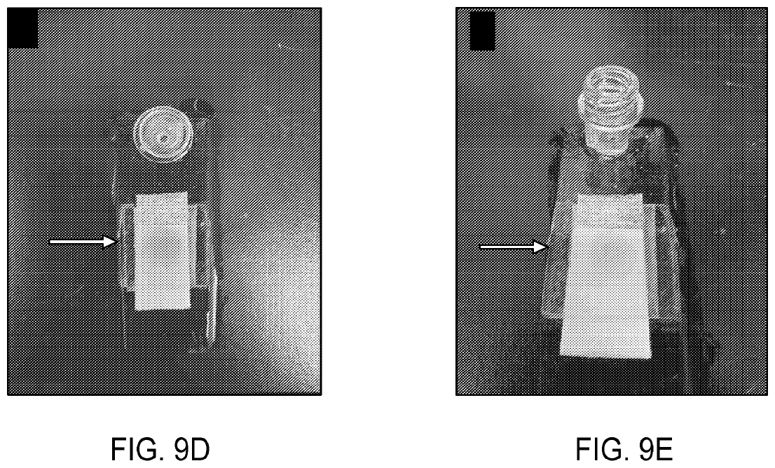

FIG. 9A shows a second capillary assembly showing a capillary pad and sealing pad inserted in a sleeve (901). The exit port of the capillary lies beneath and is sealed by sealing pad. Colored dye was introduced into the reservoir, but does not flow into the entry port of the capillary vessel. FIG. 9B shows an overhead view of FIG. 9A. FIG. 9C shows that the capillary pad and sealing pad have been pushed several millimeters into the sleeve, such that the exit port now lies beneath and is engaged with the capillary pad. Consequently, colored dye was drawn into capillary channel, flows through channel and was absorbed by filter pad. FIG. 9E shows that the capillary pad has absorbed virtually all of the colored dye introduced into the reservoir. FIG. 9D (showing a time point following that shown in FIG. 9E) shows that the reservoir is empty and the colored dye has been absorbed by the capillary pad.

### Example 3: MiCRIA Optimization for Lyme Disease and HIV

In this Example, MiCRIA was optimized for two disease applications: Lyme disease and HIV. Serum samples from an internal collection were used; for Lyme disease, the collection included approximately 6 well-characterized Lyme sera acquired from commercial suppliers, with ELISA results available for all; for HIV, approximately 2 HIV-positive sera from commercial sources were used. As controls, serum samples from healthy blood donors were used.

### Assay Protocol

The timing of incubation of serum sample and particles prior to the capillary separation step was varied between 0-5 minutes, and the volumes of each reactant were likewise varied from 0.1-5 μl.

### Capillary Vessel

Rectangular glass capillary vessels from Vitrocom were used. While the initial experiments were conducted using 3 mm×0.3 mm (volume 45 μl) and 2 mm×0.1 mm (volume 10 μl) capillaries, those skilled in the art will appreciate that other capillaries are available with a wide range of dimensions from 0.1 mm-10 mm in width and 0.010 mm-1 mm in depth and can be used in the methods, instruments, and kits described herein. A variety of the available capillaries to determine the optimal dimensions were tested.

### Optical Reader

Key parameters optimized were the neodymium magnet, excitation source and sensor. Various materials, sizes, shapes, and strengths of magnet were tested to determine the optimal specifications for capture of paramagnetic particles that will concentrate and maximize assay signal intensity. While the original prototypes were constructed using triangular magnets, which concentrate the magnet field, small magnets of diameter 2 mm and thickness 1 mm were found sufficient to capture essentially all paramagnetic particles flowing through the capillary vessel.

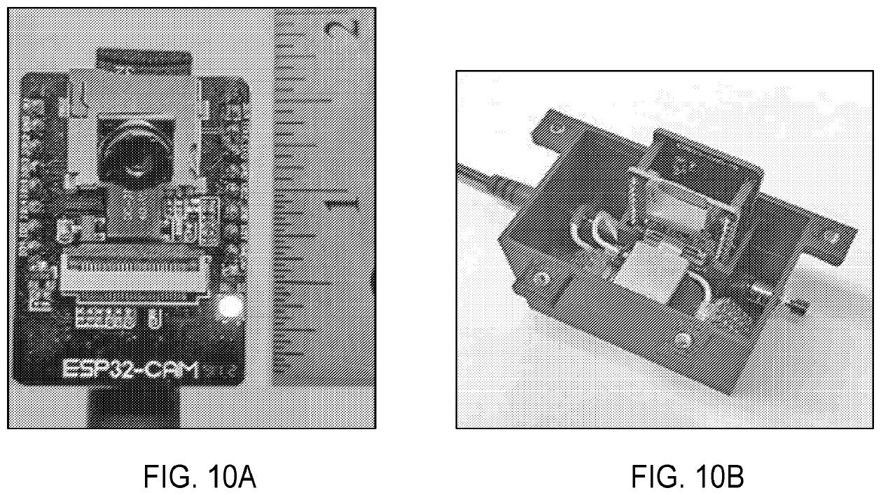

A compact, light-proof reader housing was fabricated by 3D printing, in which the magnet, UV source and sensor were mounted, and which was adapted to receive the capillary vessel (FIGS. 10A-10B). The housing was designed to allow access to the entry port to enable addition of the sample after insertion of the capillary vessel. An internal slotted mount holds the wicking (cigarette) filter against the capillary end, and was replaced for each assay run. The 365 nm flashlight used in the prototype to excite Europium as label was replaced by a 365 nm LED (Nichia, Mouser) and optical filter to remove longer wavelengths, mounted within the housing. An ESP32-CAM chip. which combines microcontroller, WiFi/Bluetooth and image processing functions with an OV2640 2 MP CMOS camera, was used to capture the fluorescent signal. As those skilled in the art will appreciate, similar chips are available as alternatives and can be used in the methods, instruments, and kits described herein. A simple laptop interface was developed to control the reader and to monitor acquired images via Bluetooth. The fluorescent signal was quantitated by the interface for determination of sensitivity and specificity. Power was provided by a USB connection.

### Evaluation

The MiCRIA assay using C6 peptide was compared with an in-house C6 peptide lateral flow test for detection of Lyme disease antibodies in a panel of 6 serum samples from Lyme disease patients. Results demonstrated detection of all 6 positive sera in the panel by both assays. Relative signal strength for each sample was also similar between assays. Thus, the MiCRIA C6 peptide Lyme assay disclosed herein is able to deliver results within 2 minutes that are equivalent to a lateral flow test using the same antigen that takes 15-20 minutes to perform.

### Example 4. Detection of Lyme-Positive Sera by MiCRIA

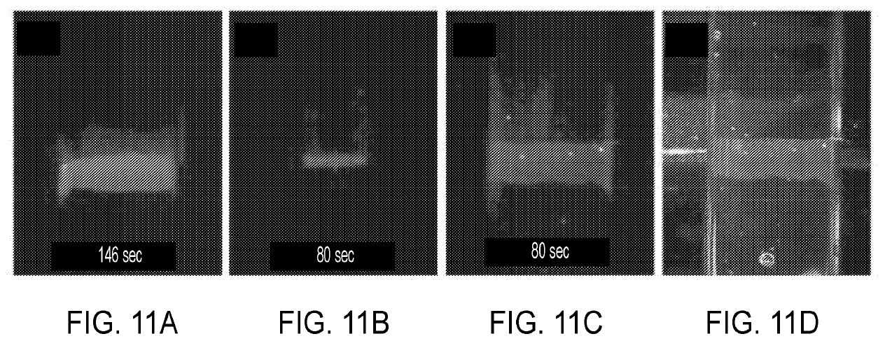

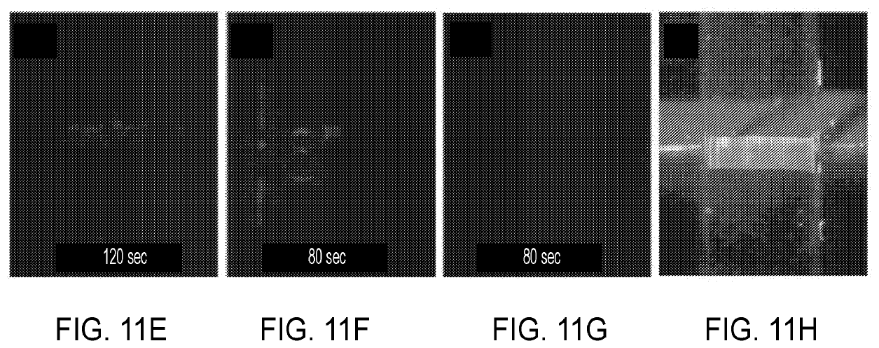

Lyme disease sera were detected using the 26 amino acid C6 peptide, derived from the Borrelia burgdorferi VlsE protein, as antigen (Liang et al., J Clin Microbiol. 1999; 37(12):3990-6). C6 peptide ELISA is a well-established assay for Lyme disease, with sensitivity in the 90-100% range and specificity ˜98-99% (Wormser et al. Diagn. Microbiol. Infect. Dis. 2013 January; 75(1):9-15; Wormser et al. Clin. Vaccine Immunol. 2008; 15(10):1519-22; Bacon et al. J. Infect. Dis. 2003; 187:1187-99). As in the HIV assay of Example 2, biotinylated C6 peptide was conjugated to streptavidin-coated Europium particles, which were used in conjunction with Protein A/G-coated Sera-Mag magnetic particles. Three well-characterized ELISA-positive sera from Lyme patients, along with three negative control sera from healthy blood donors, were tested by MiCRIA assay. One μL of each undiluted serum sample was incubated with 1 μL of Europium particles and 2 μL of magnetic particles for 1 minute, after which the mixture was processed through the capillary vessel followed by 0.5 mL of chase buffer; total assay time was <5 minutes. The three Lyme-positive sera were readily detected in the MiCRIA assay, while the three negative controls yielded no signal (FIGS. 11A-11H). FIG. 11A shows a positive sample detected at 146 seconds after introduction of the sample into the capillary. FIG. 11B shows a second positive sample detected at 80 seconds after introduction of the sample into the capillary. FIG. 11C shows a third positive sample detected at 80 seconds after introduction of the sample into the capillary. FIG. 11D shows the same sample as in FIG. 11C, but under white light rather than ultraviolet, to show the magnetic particles forming a visible band. FIG. 11E shows a negative sample detected at 120 seconds after introduction of the sample into the capillary. FIG. 11F shows a second negative sample detected at 80 seconds after introduction of the sample into the capillary. FIG. 11G shows a third negative sample detected at 80 seconds after introduction of the sample into the capillary. FIG. 11H shows the same sample as in FIG. 11G, but under white light rather than ultraviolet, to show the magnetic particles forming a visible band, which proves that the absence of a fluorescent signal in the assay of a negative sample is not due to the absence of magnetic particles. Capillary vessels of two different dimensions (3 mm×0.3 mm in FIGS. 11A, 11C, 11D, 11E, 11G and 11H, and 2 mm×0.1 mm in FIG. 11B and FIG. 11F) were used, showing the flexibility of the method.

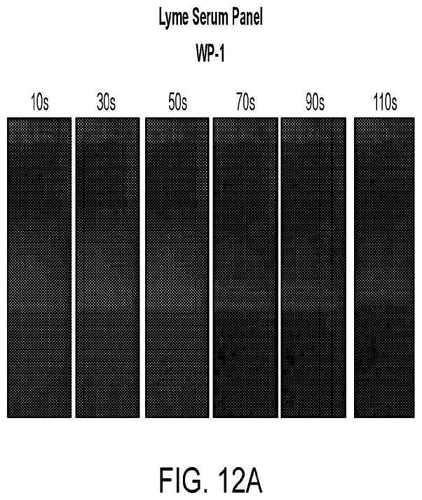

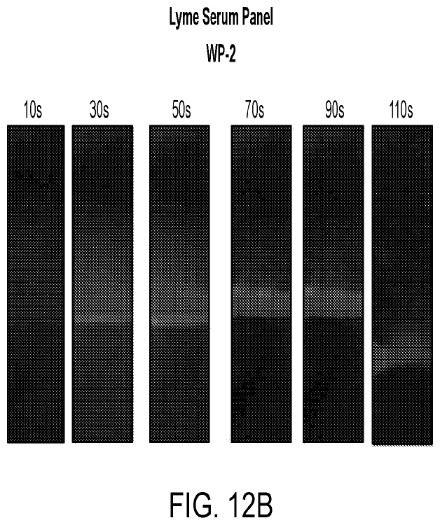

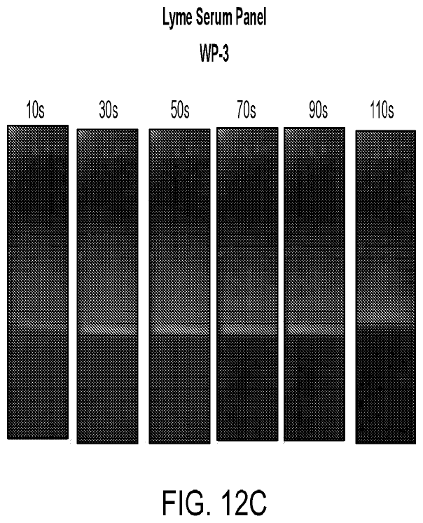

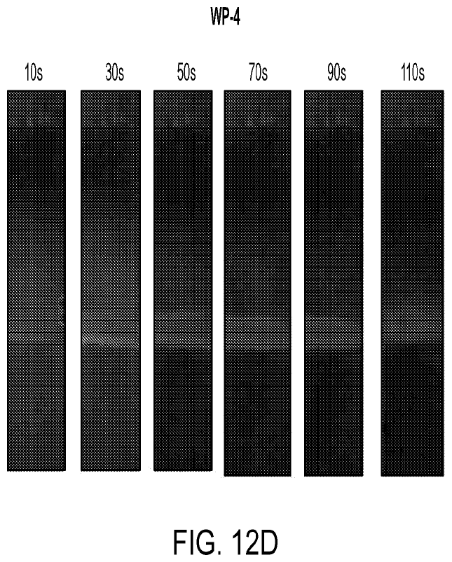

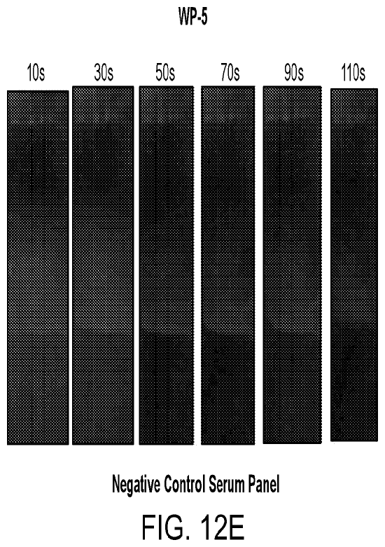

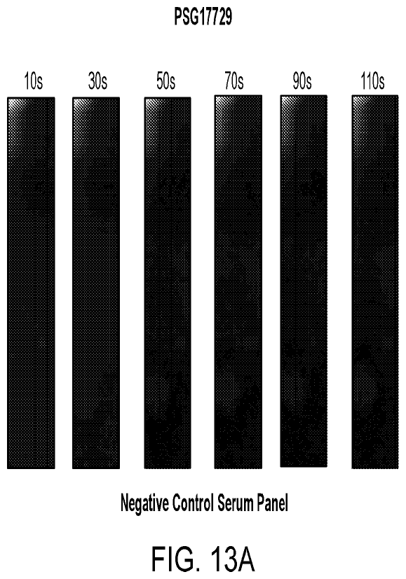

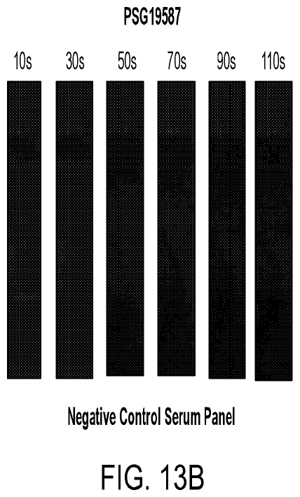

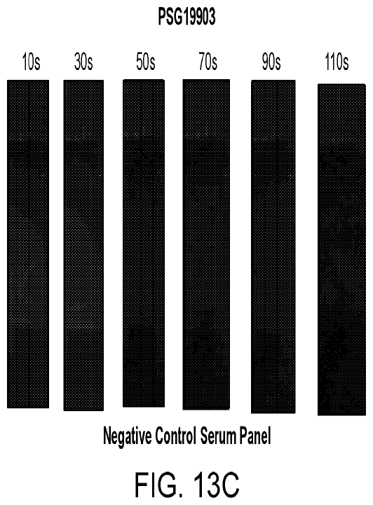

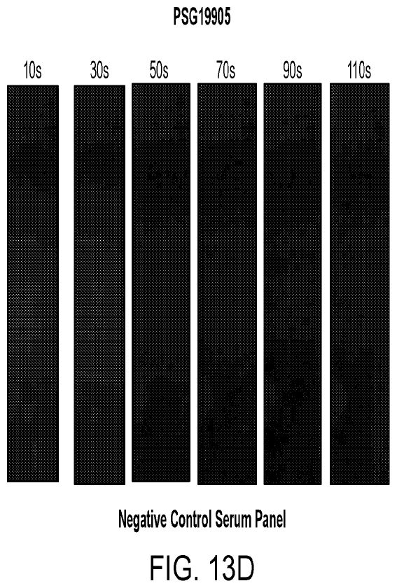

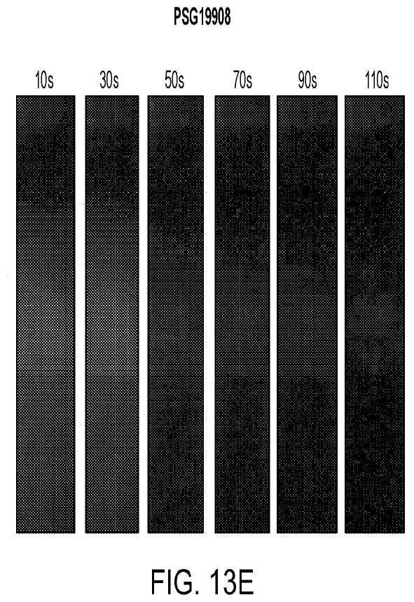

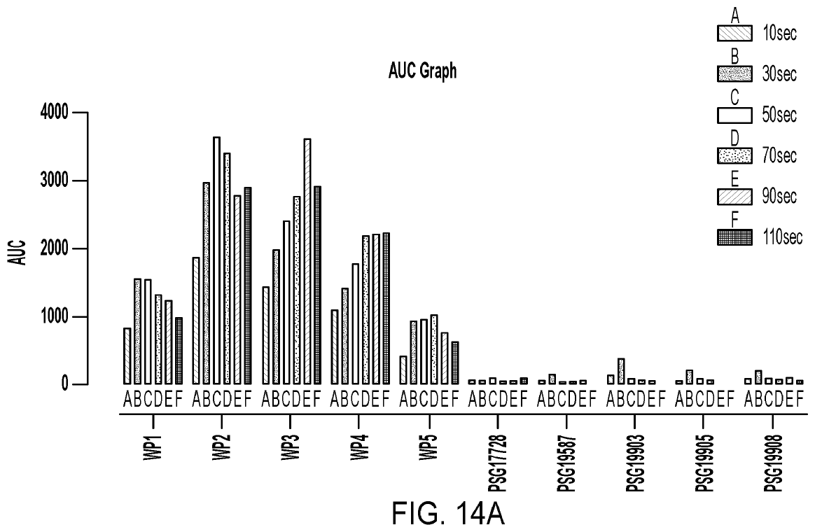

To further confirm detection of Lyme disease positive sera by MiCRIA, a panel of 5 serum samples from Lyme disease patients (WP-1 through WP-5) and 5 serum samples from healthy blood donors was tested. Five μL of each serum sample was mixed with 45 μL diluted Europium beads conjugated to C6 peptide and incubated for 1 minute. The C6 peptide is a peptide derived from the VlsE (Variable Large Protein) protein, which is a lipoprotein located on the surface of the Lyme Disease spirochete. One μL paramagnetic particles conjugated to Protein A/G (a recombinant fusion protein that combines the IgG binding domains of both Protein A and Protein G) were added to the mixture and incubated for 1 minute. The solution containing the serum sample and both particles was introduced into the capillary device followed by about 200 μL wash buffer in two successive aliquots. Images of the fluorescent signal in the capillary were captured at 10 second intervals. Fluorescent signals at 50-70 seconds from the start of the capillary flow were used for assay interpretation. FIGS. 12A-12E (WP-1, WP-2, WP-3, WP-4, and WP-5, each representing a sample derived from a Lyme disease positive subject) show results from Lyme disease positive samples. FIGS. 13A-13E (PSG17729, PSG19587, PSG19903, PSG19905, and PSG19908, each representing a sample derived from a Lyme disease negative subject) show results from a negative control serum panel. Red lines in FIGS. 12A-12E indicate detection of Lyme disease positive sera, and demonstrate visually that MiCRIA is capable of detecting Lyme disease positive samples, while FIGS. 13A-13E demonstrate visually that MiCRIA is capable of distinguishing Lyme disease negative from positive samples.

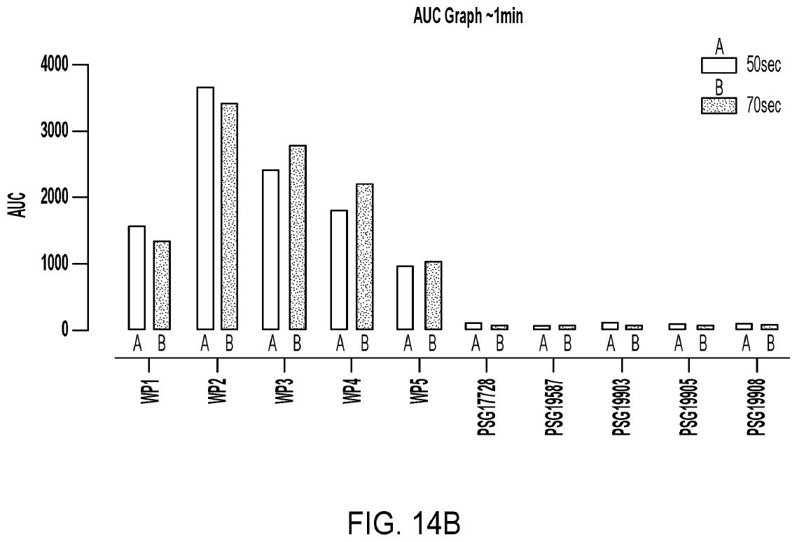

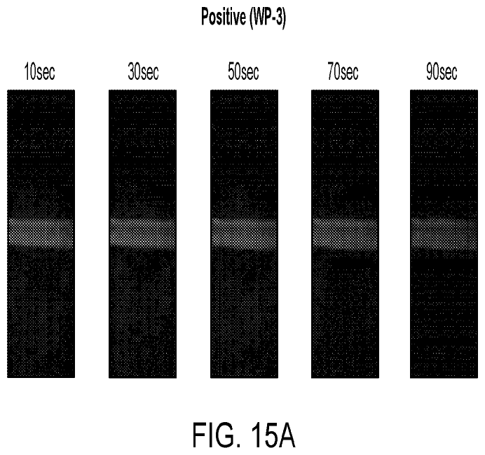

Based on processing the images shown in FIGS. 12A-12E and FIGS. 13A-13E, Area Under the Curve (AUC) was calculated for Lyme disease positive and Lyme disease negative sera, as shown in FIGS. 14A-14B. FIG. 14A shows the AUC calculated for each time point for each serum sample. FIG. 14B shows the AUC for the 50 sec. and 70 sec. time points, either of which may be used for interpretation of assay results. All Lyme positive sera show AUC≥1000, while negative sera show negligible AUC.

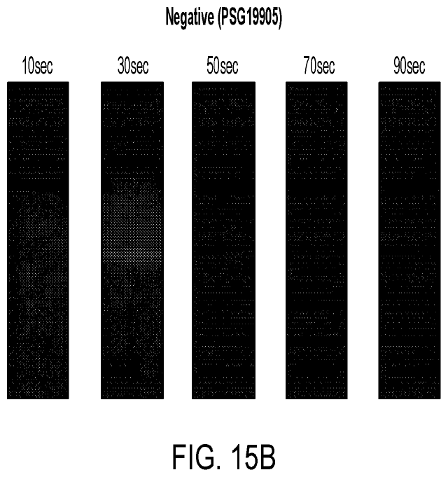

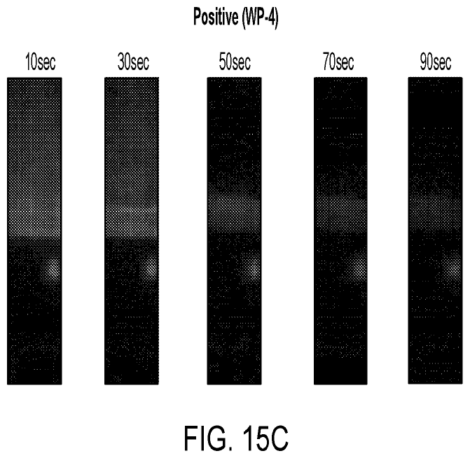

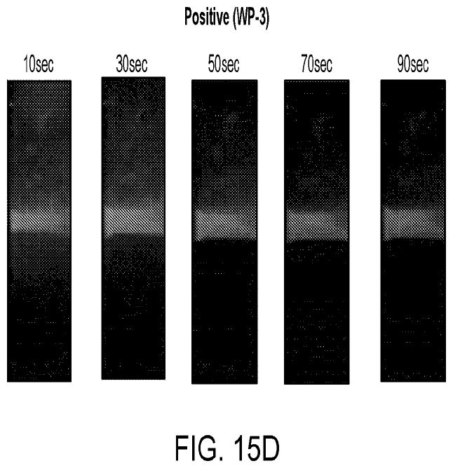

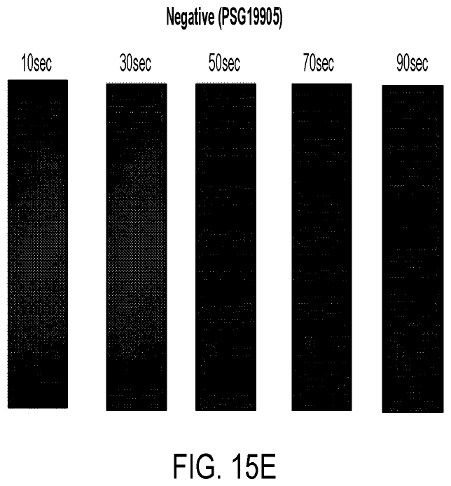

In addition to screening Lyme disease positive and negative serum samples, whole blood samples spiked with Lyme disease positive and Lyme disease negative samples were screened. Whole blood was obtained by finger prick from a volunteer. Lyme disease samples were spiked into the whole blood at a 1:1 dilution. Samples were spiked with either WP-3 (positive) or PSG19905 (negative) serum. FIG. 15A shows a positive signal at 50-70 seconds for a whole blood sample spiked with Lyme positive serum. FIG. 15B shows a negative signal for a whole blood sample spiked with negative serum. These results demonstrate that whole blood can be tested in the capillary device without a requirement for prior removal of red blood cells or separation of serum or plasma. FIGS. 15C-15D show additional positive results. FIG. 15E shows an additional negative result.

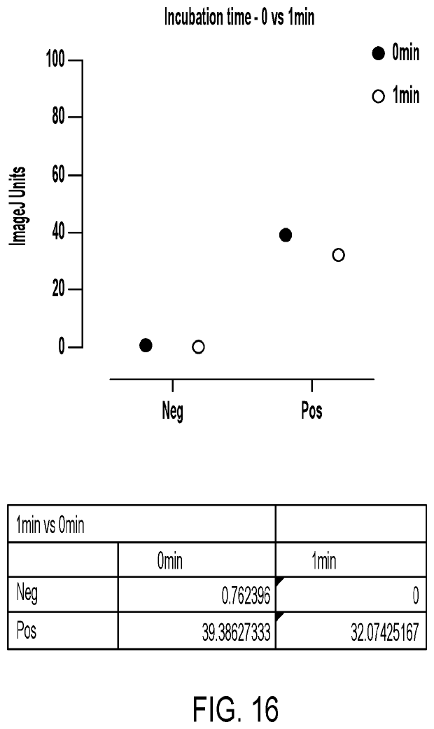

To determine the length of incubation time required to detect Lyme disease positive sera, samples were either incubated for 0 or 60 seconds prior to being added to the MiCRIA instrument. As shown in FIG. 16, no difference was observed between signal intensity in assays using 0 or 60 seconds, demonstrating that MiCRIA assays can provide consistent results even with an incubation time less than 1 minute or without incubation at all. FIG. 16 (top) demonstrates that MiCRIA can detect both positive results and negative results with or without incubation. FIG. 16 (bottom) shows the quantitative data that is visualized in FIG. 16 (top).

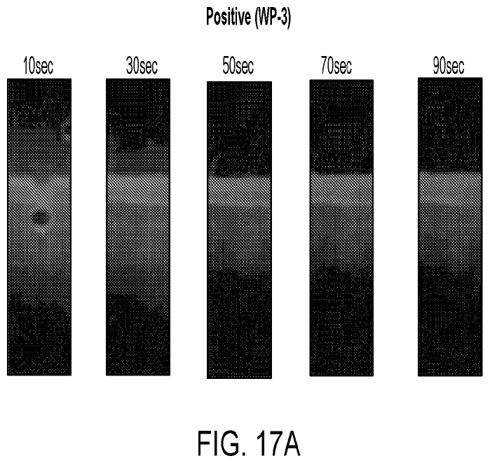

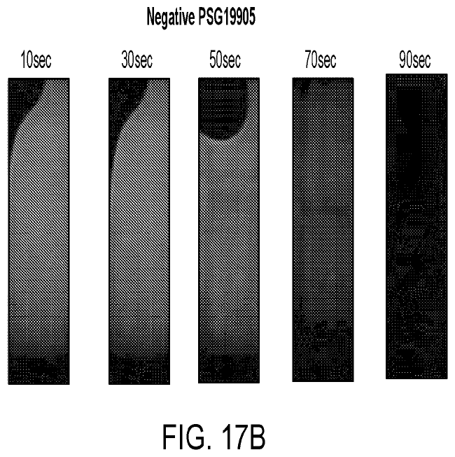

In addition to whole blood, the MiCRIA assay was tested using dried blot samples. Aliquots of fresh whole blood spiked with Lyme positive or negative serum were dried on a filter to generate mock dried blood spots (DBS). To prepare the DBS, 10 μL of whole blood and 10 μL of serum sample (Lyme Positive WP3 and Negative Control PSG19905) were mixed and added to the dried blood spot. The DBS was then used in the MiCRIA protocol. 80 μL of Phosphate Buffered Saline with TWEEN® 20 (PBST) with 0.1% Bovine Serum Albumin (BSA) was added to the DBS and vortexed for 1 min. The sample was then pulse centrifuged and 40 μL of DBS solution was added to 60 μL of PBST with 0.1% BSA. The Europium solution was prepared at a 1:500 concentration using PBST with 0.1% BSA. 5 μL of the sample was combined with 45 μL of the Europium solution without incubating. 2 μL of Magnetic beads were then added to the solution without incubating, followed by addition of 200 μL of PBST 0.1% BSA. This final solution was then added to the capillary. The positive results in FIG. 17A and the negative results in FIG. 17B demonstrate that Lyme disease antibodies can be detected in dried blood spots with the MiCRIA assay.

Taken together, these results demonstrate that the MiCRIA assay is able to rapidly detect Lyme disease positive sera without the need for pre-incubation or with minimal (e.g., one minute) incubation.

### Example 5: Detection of HIV Positive Sera and COVID-19 Positive Sera by MiCRIA

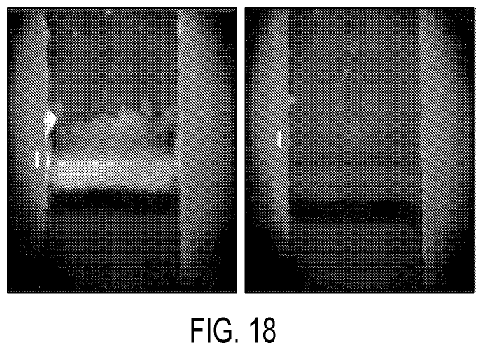

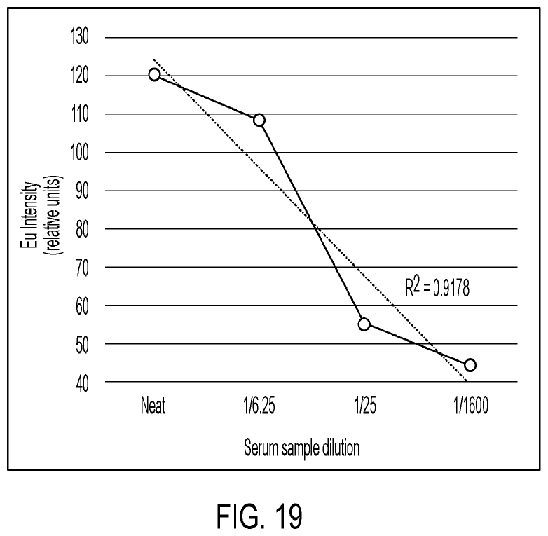

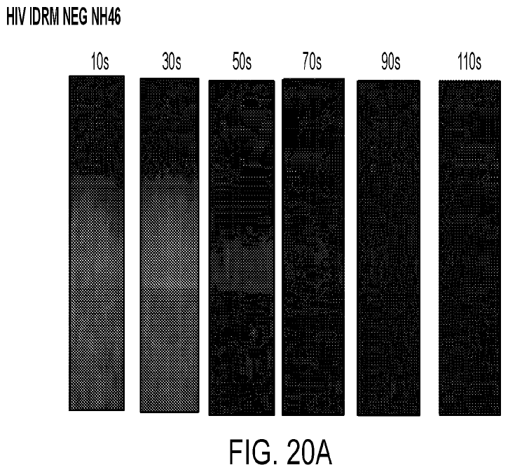

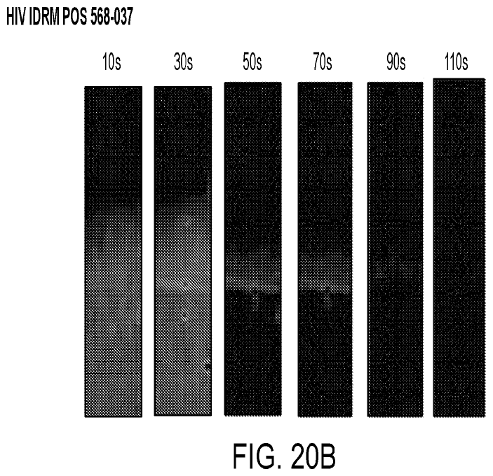

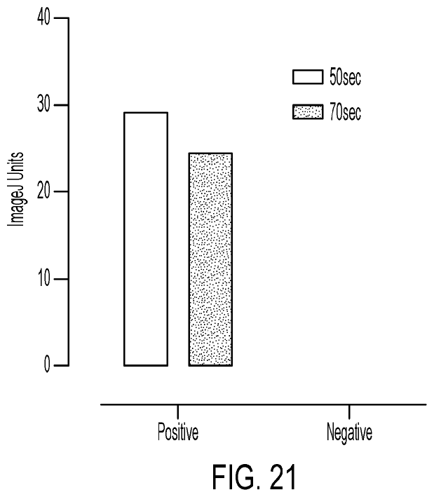

The protocol described in Example 4 was used to detect HIV-positive samples via the MiCRIA assay. HIV-positive and negative (by ELISA) serum samples were tested by MiCRIA assay for reactivity to IDR-m, a 34 amino acid immunodominant peptide antigen derived from the gp41 protein (Dorn et al., J Clin Microbiol. 2000; 38(2):773-80; Wei et al., AIDS Res Hum Retroviruses. 2010). N-terminal biotinylated IDR-m was conjugated to streptavidin-coated Europium particles (Cytiva) (Fluoro-Max™ Fluorescent Carboxylate-Modified Particles), and 1 μm Sera-Mag Protein A/G-coated magnetic particles were obtained from Cytiva (Sera-Mag SpeedBeads and Sera-Mag Carboxylate-Modified Magnetic Particles—GE Healthcare Life Sciences). As shown in FIG. 18, the bright Europium signal was readily detected in the MiCRIA assay with HIV-positive serum (left), while negligible signal was seen with the HIV-negative serum (right). Testing serial dilutions of the HIV-positive serum by MiCRIA assay showed a typical dilution curve as shown in FIG. 19. With no assay optimization, a signal was detected down to a dilution of 1:25. FIGS. 20A-20B demonstrate additional detection of HIV-negative and positive samples by the MiCRIA assay. FIG. 21 shows quantification of the results in FIGS. 20A-20B. A human serum sample containing HIV antibodies was detected in the MiCRIA capillary assay while a negative control serum sample yielded no detectable signal, as shown in FIG. 21.

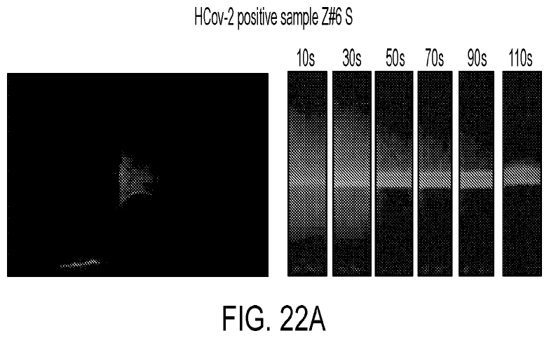

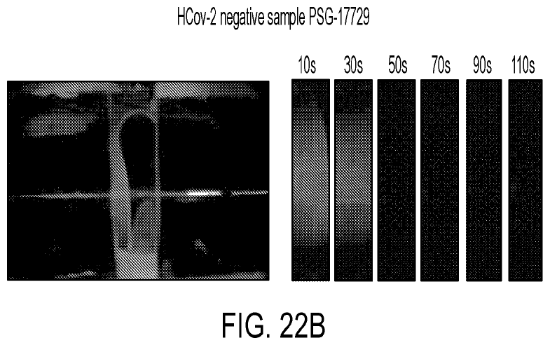

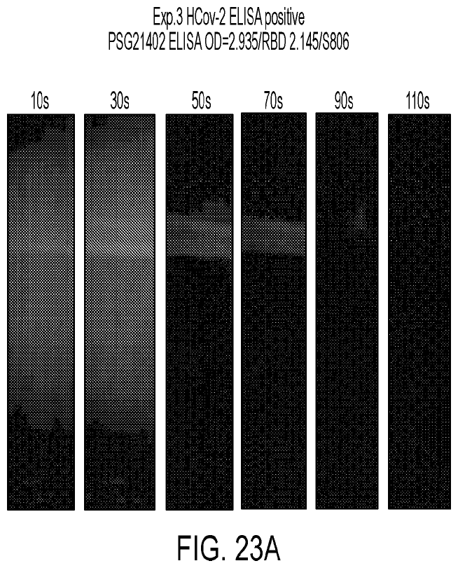

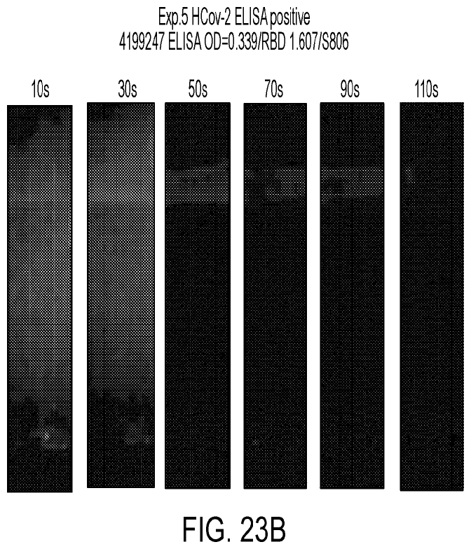

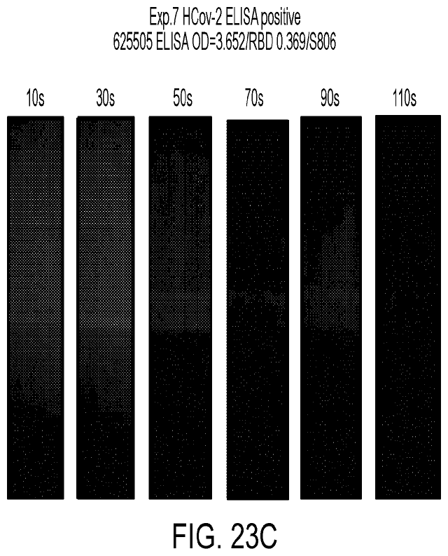

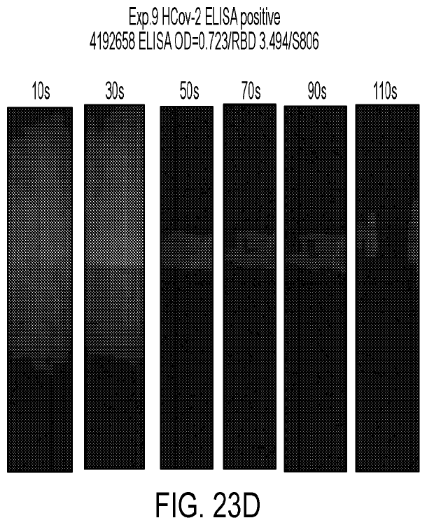

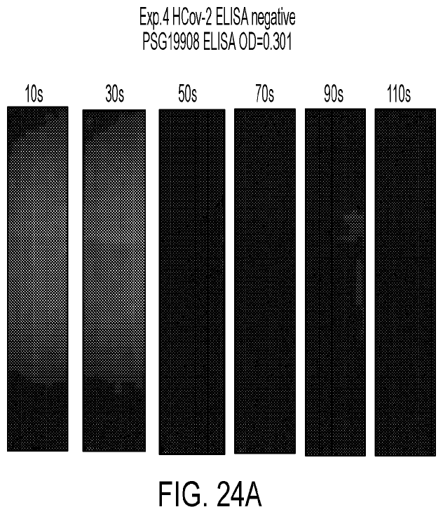

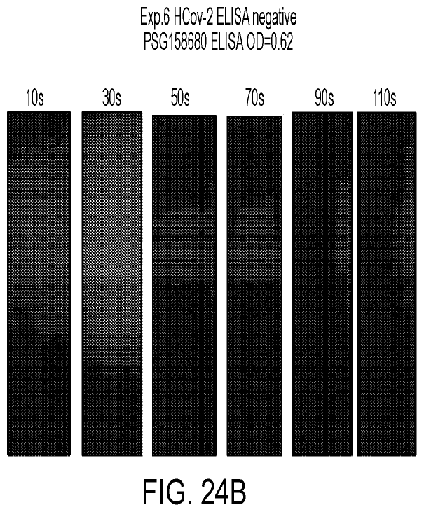

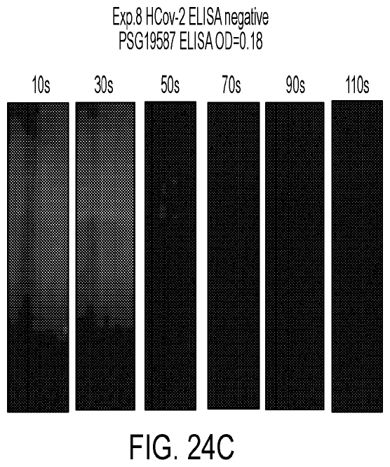

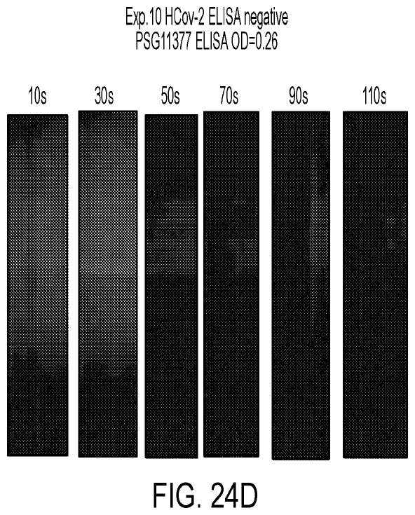

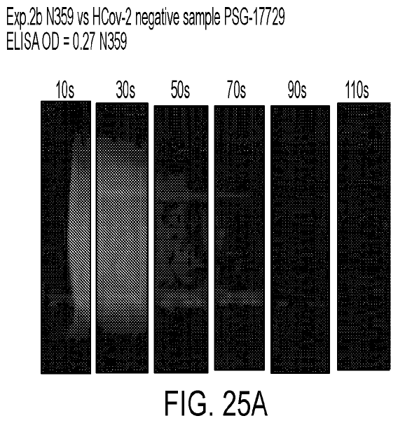

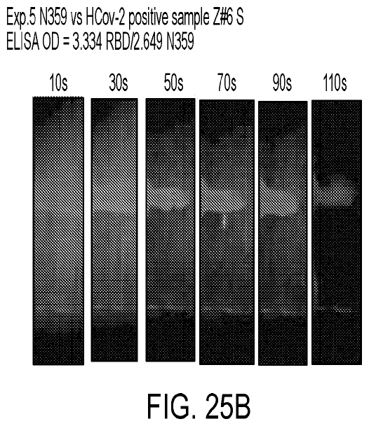

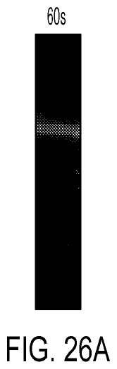

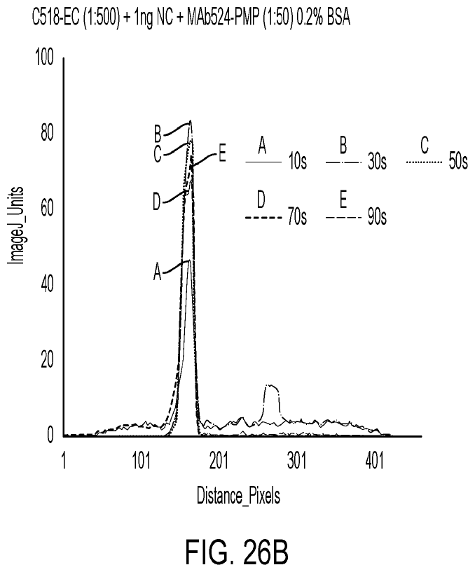

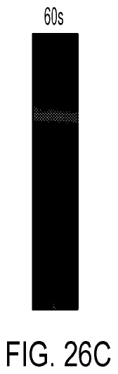

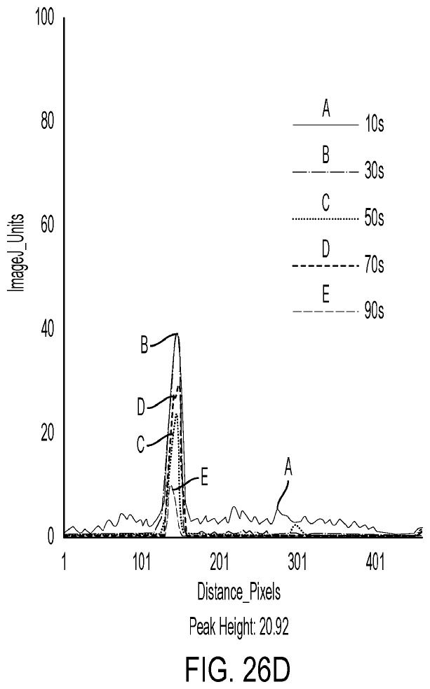

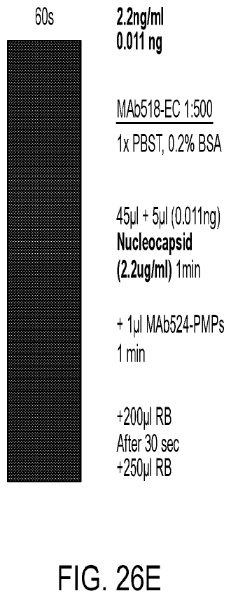

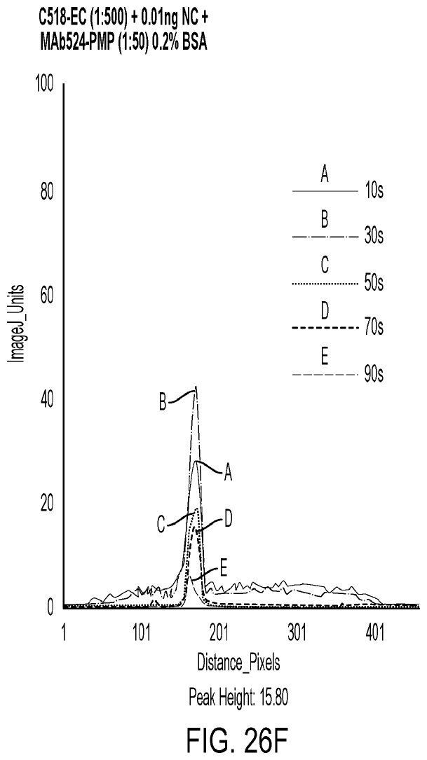

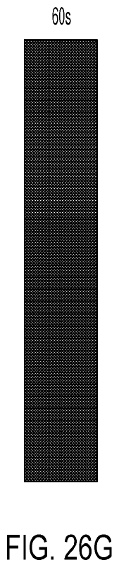

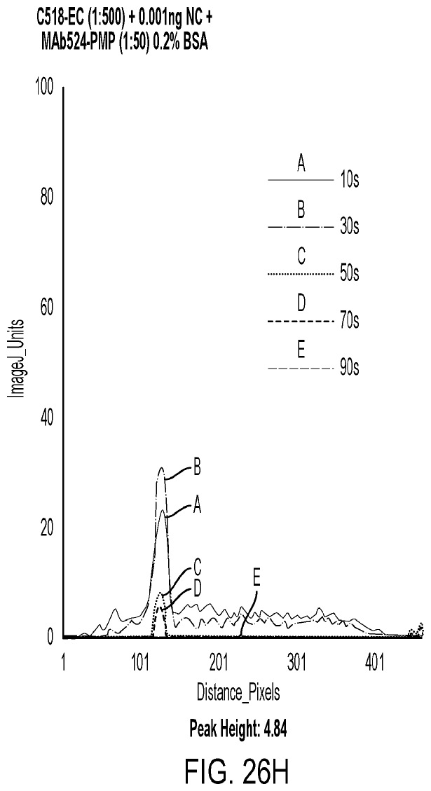

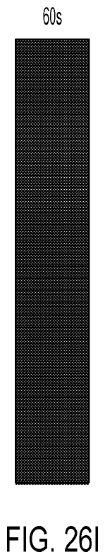

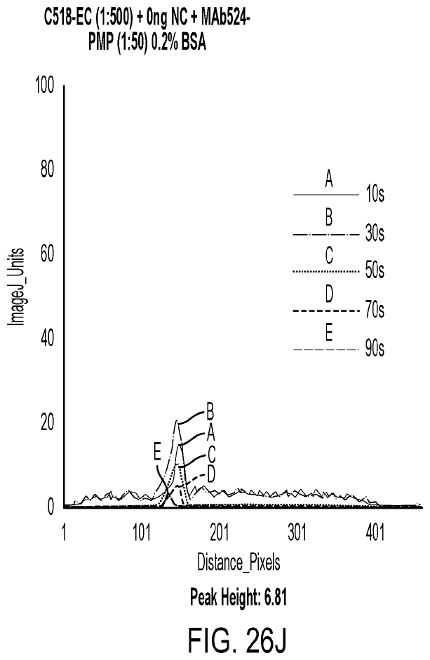

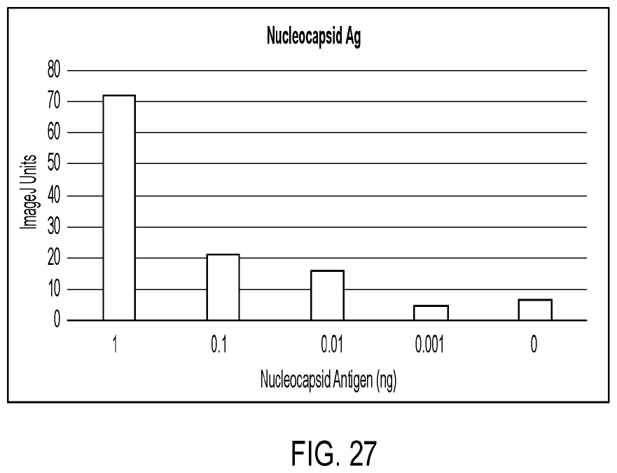

Similarly, the MiCRIA assay was used to detect COVID-19 antibodies to the receptor binding domain (“RBD”) protein (FIGS. 22A-22B), to a peptide derived from the spike protein (FIGS. 23A-23D and FIGS. 24A-24D), and to a peptide derived from the nucleocapsid protein (FIGS. 25A-25B) in human serum samples, as shown in FIGS. 22A-22B, FIGS. 23A-23D, FIGS. 24A-24D, and FIGS. 25A-25B. In addition, FIGS. 26A-26H demonstrate the sensitivity of the MiCRIA assay by showing detection of COVID-19 nucleocapsid antigen spiked into buffer at concentrations ranging from 1 ng to 0 ng. A positive result was measured at 60 seconds, indicating detection of COVID-19 nucleocapsid at concentrations from 1 ng down to 0.01 ng. Readings that generated a peak height greater than 10 were considered positive and readings that generated a peak height less than 10 were considered negative. These data indicate that the MiCRIA assay is capable of nucleocapsid antigen detection at low concentrations such as are found in nasopharyngeal swab samples taken from patients infected with COVID-19.

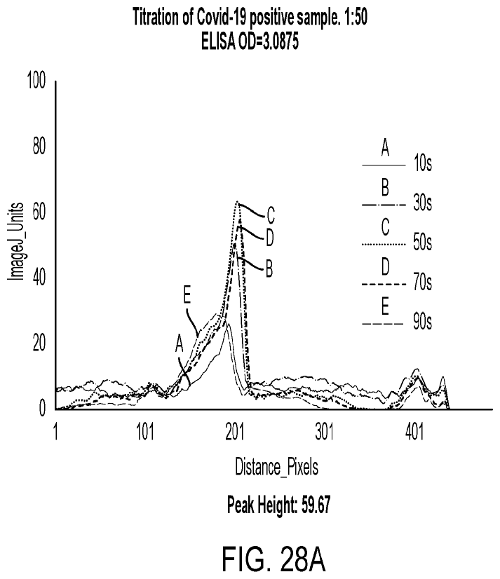

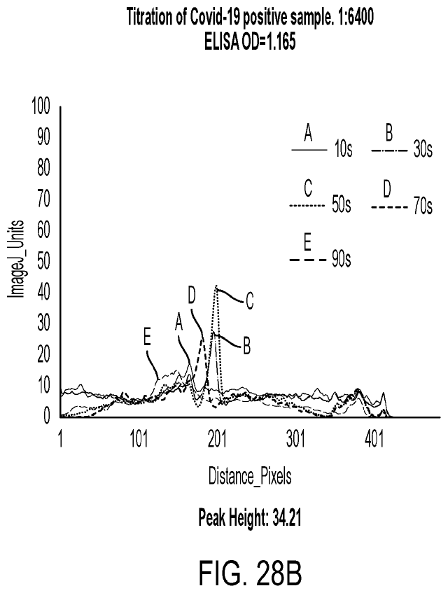

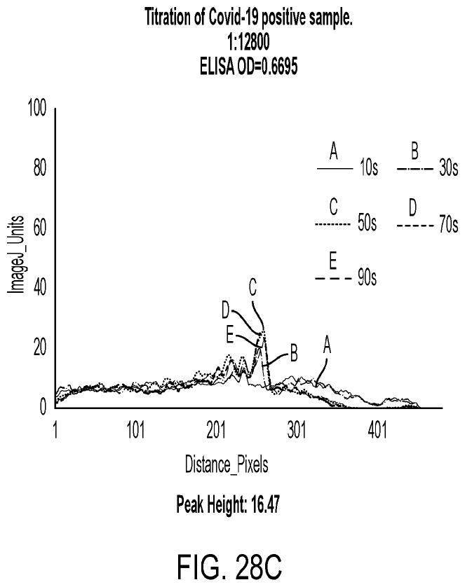

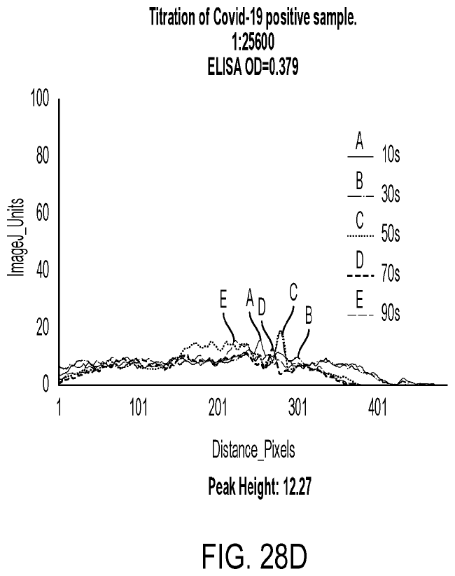

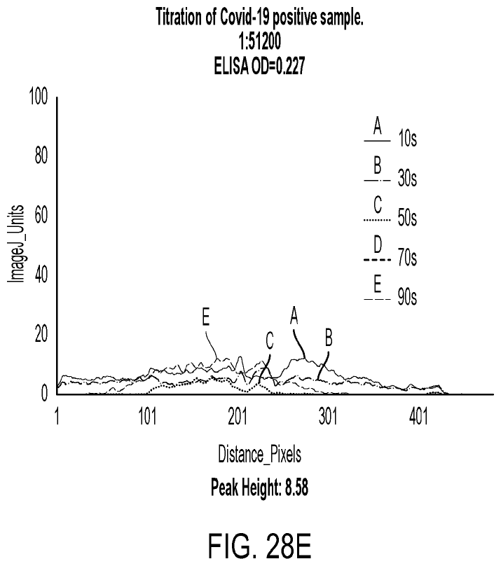

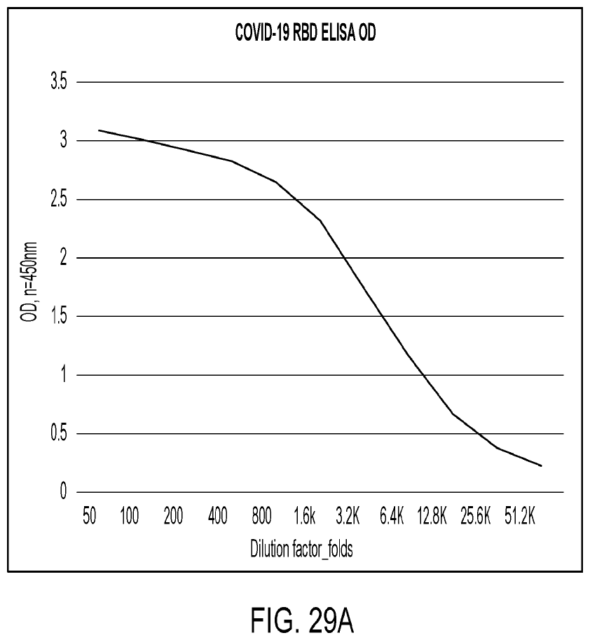

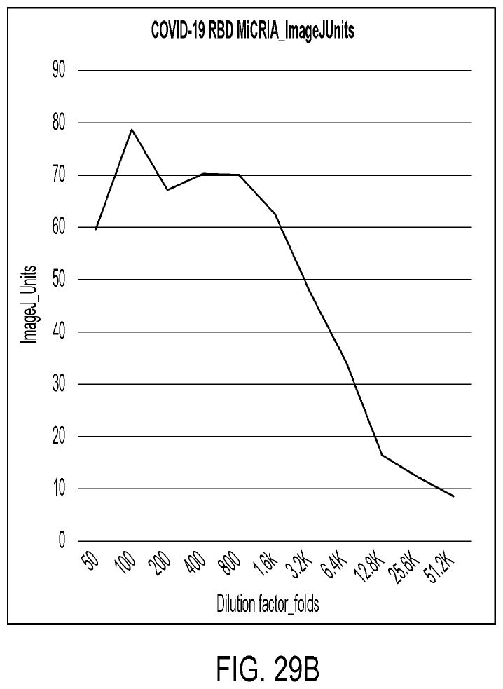

MiCRIA results were also compared to ELISA results (FIGS. 28A-28E, FIGS. 29A-29B). This comparison demonstrates that the MiCRIA method can be used to detect COVID-19 antibodies in blood samples from patients infected with the virus, similarly to ELISA, which is a surprising finding given that ELISA relies on enzyme amplification while MiCRIA has no amplification step.

Taken together, the results described herein indicate that detection of analytes by the MiCRIA assay is simpler and faster than other assays, including lateral flow. While other assays require up to 15 minutes to provide results, the MiCRIA assay can provide results in as few as 2 minutes. This increased turnaround time may be critical in emergency situations or in situations in which many tests need to be completed in a short amount of time.

### Example 6: Exemplary MiCRIA Assay Assembly

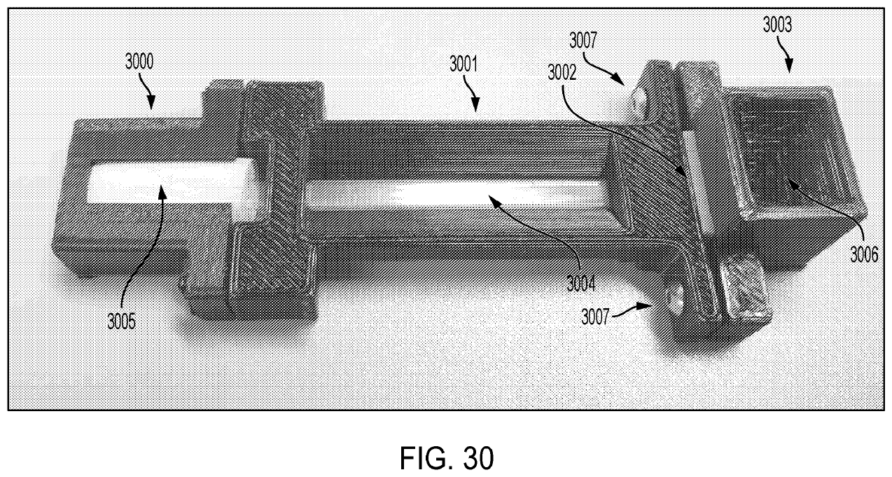

In this Example, the MiCRIA assay assembly shown in FIG. 30 was constructed utilizing 3D printing. The assembly was constructed from three separate 3D-printed components: (1) a capillary housing 3001 adapted to receive a capillary vessel (shown with a capillary vessel 3004 in place in FIG. 30); (2) a pad housing 3000 adapted to receive a capillary pad (shown with a capillary pad (cigarette filter 3005) in place in FIG. 30); and (3) a reservoir chamber 3003 including a reservoir cavity 3006; where the capillary housing 3001 is affixed to the pad housing 3000 on one end by two plastic pins (not shown) and to the reservoir chamber 3003 on the opposite end by two metal screws 3007. As shown in FIG. 30, a rubber septum 3002 with an aperture through which the capillary vessel is inserted is held in place between the capillary housing 3001 and the reservoir chamber 3003 sufficient to form a liquid-tight seal such that sample inserted into the sample reservoir cavity 3006 flows into the capillary vessel 3004 without leaking. The capillary vessel 3004 in FIG. 30 was inserted into capillary housing 3001 through apertures in opposite sides of the capillary housing 3001 and through the aperture in the rubber septum 3002 such that the capillary vessel 3004 is in fluid communication with both the capillary pad 3005 and the reservoir cavity 3006.

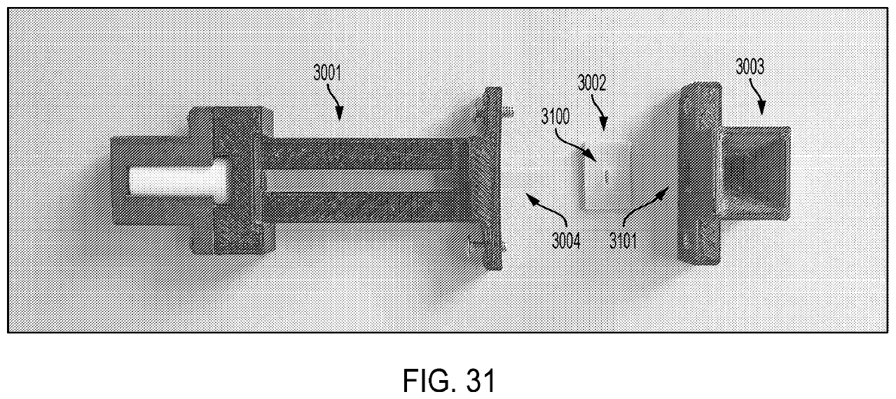

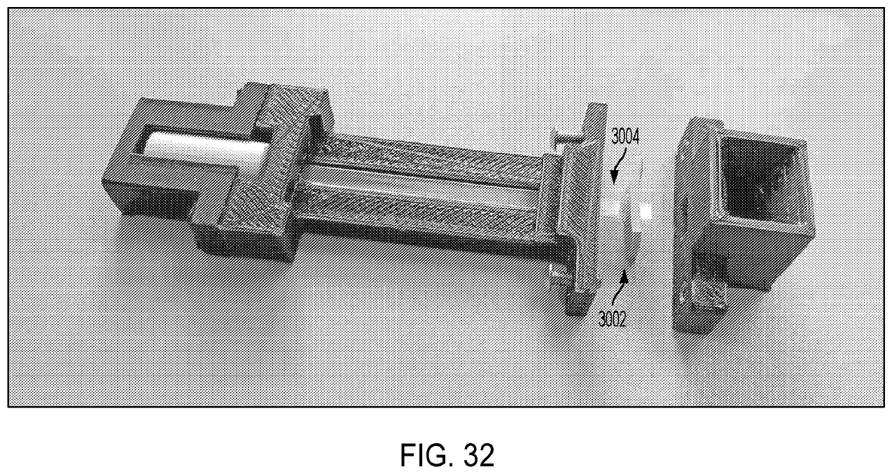

FIG. 31 shows an exploded top view of the exemplary MiCRIA assay assembly shown in FIG. 30, where the reservoir chamber 3003 is disconnected from the capillary housing 3001. The aperture 3100 in the rubber septum 3002 and the aperture 3101 in the reservoir chamber 3003 adapted to receive the capillary vessel 3004 are shown.

FIG. 32 shows a perspective view of FIG. 31, where the capillary vessel 3004 can be seen inserted through the aperture in the rubber septum 3002.

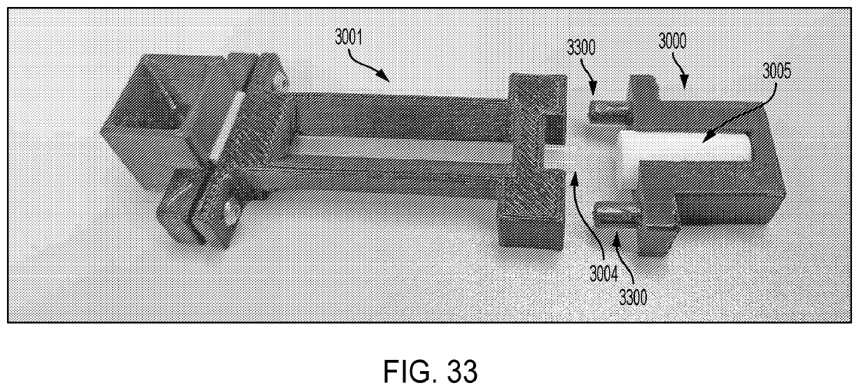

FIG. 33 shows an exploded perspective view of the exemplary MiCRIA assay assembly shown in FIG. 30, where the pad housing 3000 is disconnected from the capillary housing 3001 The two plastic pins 3300 can be seen. Not shown are the corresponding apertures in the capillary housing 3002 adapted to receive the plastic pins 3300, sufficient to hold the capillary vessel 3004 in fluid communication with the capillary pad 3005. The use of plastic pins allows for facile removal of the capillary housing 3002 for replacement of consumable components, such as the capillary pad 3005 and the capillary vessel 3004.

### Example 7: Exemplary MiCRIA Housing Assembly

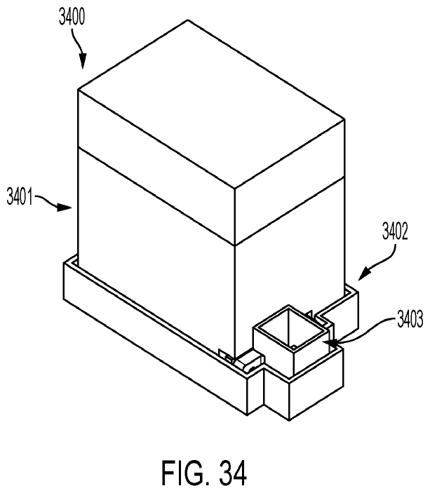

This Example describes an exemplary MiCRIA housing assembly as shown in FIGS. 34-40. The MiCRIA housing assembly of FIG. 34 is adapted to receive a diagnostic instrument. In particular, the MiCRIA housing assembly is adapted to receive one or more of the diagnostic instrument, capillary vessel, and the MiCRIA assay assembly described herein. The MiCRIA housing assembly is also adapted to facilitate any one of the methods described herein. The MiCRIA housing assembly is adapted to receive any one of the kits described herein.

The MiCRIA housing assembly of FIG. 34 comprises a top portion (3400 and 3401) and a bottom portion (3402). The top portion can be detached from the bottom portion as a single piece or as two separate pieces (top-upper, 3400; top-lower, 3401). The MiCRIA housing assembly is adapted to receive a MiCRIA assay assembly (not fully depicted) and the reservoir chamber (3403) of the MiCRIA assay assembly is adapted to be accessible from the exterior portion of the MiCRIA housing assembly.

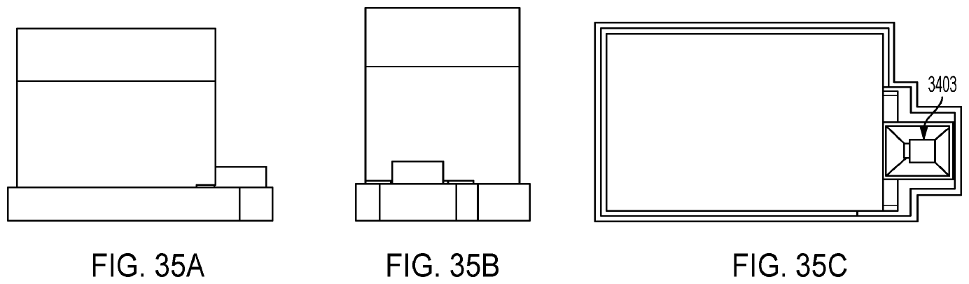

FIG. 35A shows a side view of the of the MiCRIA housing assembly and FIG. 35B shows a front view of the MiCRIA housing assembly. FIG. 35C shows a top view of the MiCRIA housing assembly with the reservoir chamber (3403) of the MiCRIA assay assembly accessible from the exterior of the MiCRIA housing assembly, allowing a user to insert a sample and reagents into the MiCRIA assay assembly from outside the MiCRIA housing assembly such that the assay is conducted in a substantially light-tight environment.

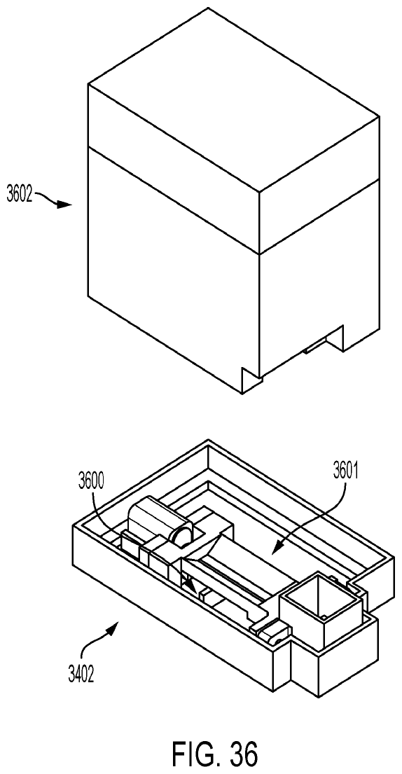

The top portion (3602) of the MiCRIA housing assembly is shown detached from the bottom portion (3402) in FIG. 36. The bottom portion (3402) holds a magnet (3600) and the MiCRIA assay assembly (3601). The magnet and MiCRIA assay assembly can be removed from the MiCRIA housing assembly or they can be built into the MiCRIA housing assembly. Where the MiCRIA assay assembly is build into the MiCRIA housing assembly, the consumable parts (e.g., capillary pad, capillary body) can be user-replaceable.

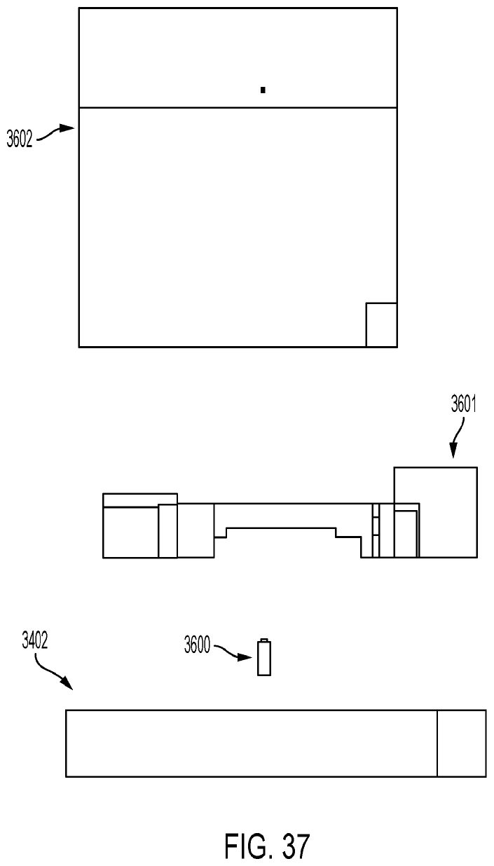

FIG. 37 shows an exploded front view of the top portion (3602) separated from the bottom portion (3402) with the magnet (3600) and MiCRIA assay assembly (3601) removed from the MiCRIA housing assembly.

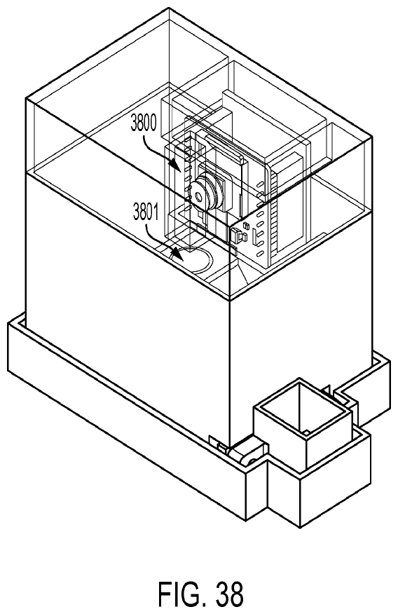

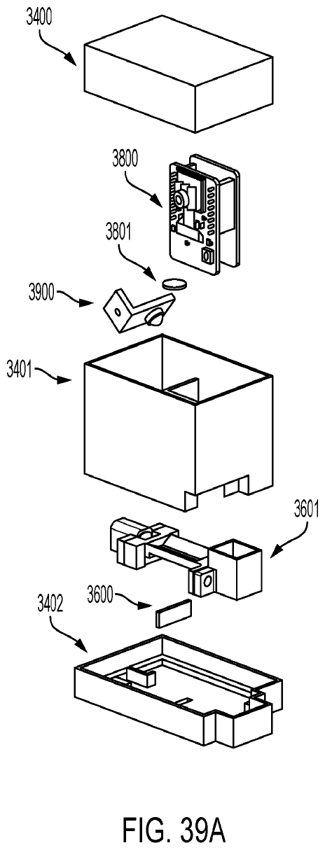

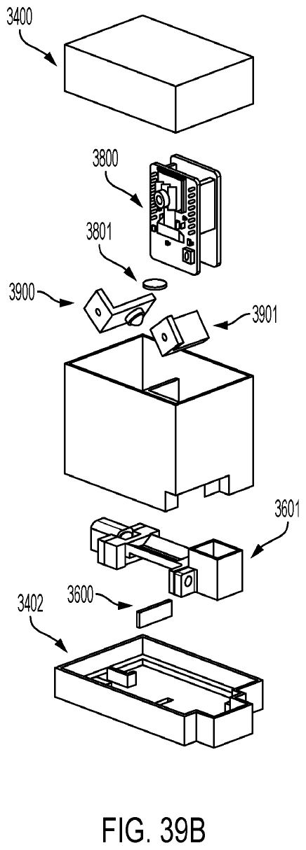

The top portion of the MiCRIA housing assembly is adapted to hold or be affixed to one or more cameras. FIG. 38 shows a camera (3800) affixed to the top portion and an optional filter disk (3801). The optional filter disk filters light derived from sources other than the assay such that only wavelengths emitted from the MiCRIA assay assembly pass through the filter disk and are detected by the camera. The top portion is transparent for demonstration purposes. The top portion is also adapted to hold or be affixed to one or more excitation sources. FIG. 39A shows an exploded view of the MiCRIA housing assembly with the top portion (further divided into a upper and lower top portions) separated from the bottom portion, the cameras separated from the top portion, the magnet and MiCRIA assay assembly separated from the bottom portion, and an excitation source (3900) separated from the top portion. FIG. 39B demonstrates that the MiCRIA housing assembly can house a first excitation source (3900) and a second excitation source (3901).

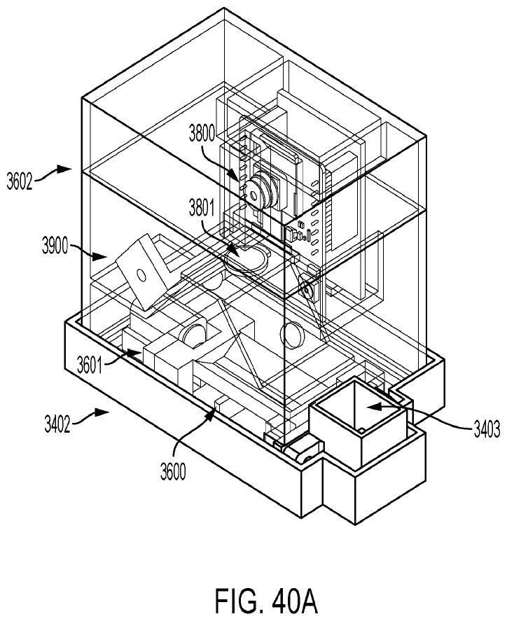

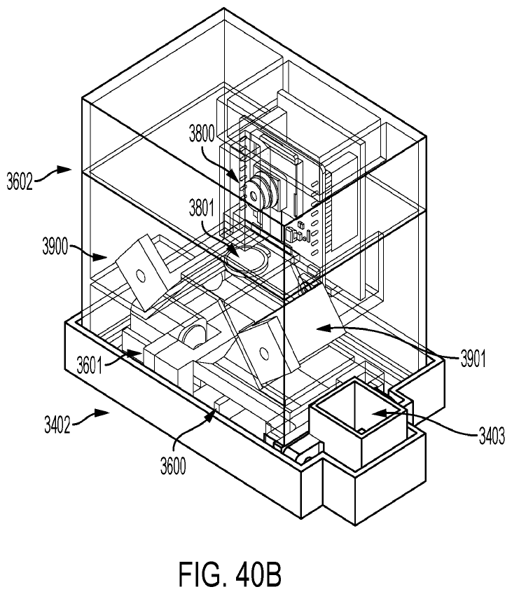

FIG. 40A shows a fully assembled MiCRIA housing assembly, with transparent top portion (3602) and bottom portion (3402) for demonstration purposes. A top portion (3602) is attached to a bottom portion (3402), forming a substantially light-tight seal. A magnet (3600) and the MiCRIA assay assembly (3601) are each affixed to the bottom portion (3402) of the MiCRIA assay assembly. The MiCRIA assay assembly (3601) is inside the substantially light-tight MiCRIA housing assembly while the reservoir chamber (3403) of the MiCRIA assay assembly (3601) is accessible from outside the MiCRIA housing assembly. A camera (3800) is affixed to the top portion (3602) of the MiCRIA housing assembly and one excitation source (3900) is affixed to the top portion (3602) of the MiCRIA housing assembly.

FIG. 40B shows the same assembly as FIG. 40A with the addition of a second excitation source (3901).

### Example 8: Comparison of MiCRIA, ELISA, and Lateral Flow

This Example describes experiments comparing the limit of detection, sensitivity and specificity, of MiCRIA, ELISA, and Lateral Flow.

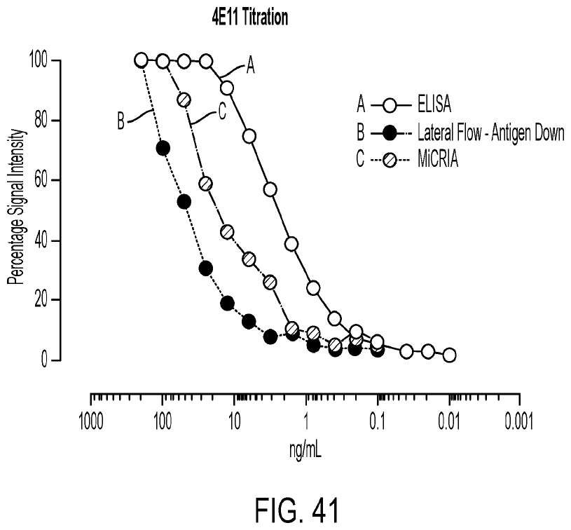

To compare the sensitivity of MiCRIA, ELISA, and lateral flow, negative serum samples were spiked with Cs1-specific monoclonal antibodies (4E11). Cs1 is a Liver Fluke antigen. FIG. 41 demonstrates that MiCRIA has an improved limit of detection (sensitivity) when compared to a lateral flow rapid test.

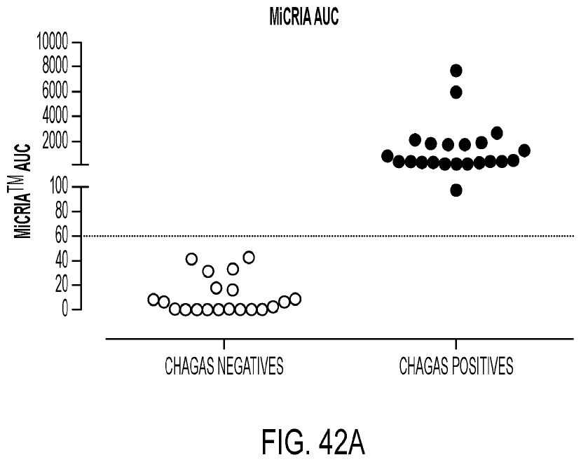

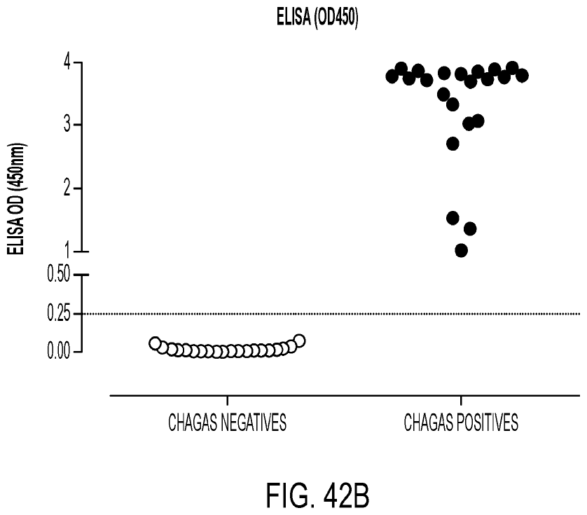

To compare MiCRIA directly to ELISA, 21 Chagas disease-positive samples and 20 negative samples were analyzed by MiCRIA and ELISA (FIGS. 42A-42B). A 2-min rapid MiCRIA assay showed 100% sensitivity and 100% specificity (FIG. 42A). Results from the MiCRIA assay were comparable to a 4 hour plus overnight ELISA assay (FIG. 42B). The results demonstrate that MiCRIA as disclosed herein provides a substantial time advantage over ELISA methods with comparably high sensitivity and specificity.

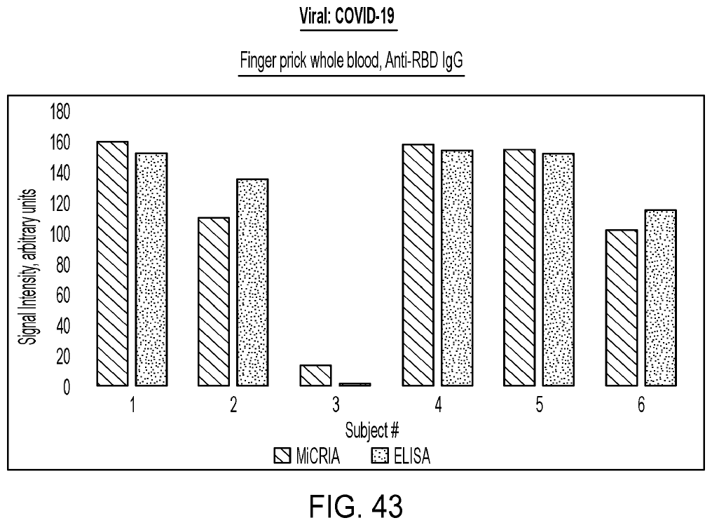

To further confirm the utility of the MiCRIA assay for the detection of various antigens, MiCRIA sensitivity was compared to lateral flow and ELISA in the context of three separate infections (FIGS. 43-46).

Finger prick whole blood samples from SARS-CoV-2-positive (anti-RBD IgG) samples were analyzed using either MiCRIA or ELISA (FIG. 43). The MiCRIA assay was run for 2 minutes while the ELISA assay was run for 2-3 hours. FIG. 43 demonstrates that the rapid MiCRIA assay has comparable sensitivity to a 2-3 hour ELISA assay.

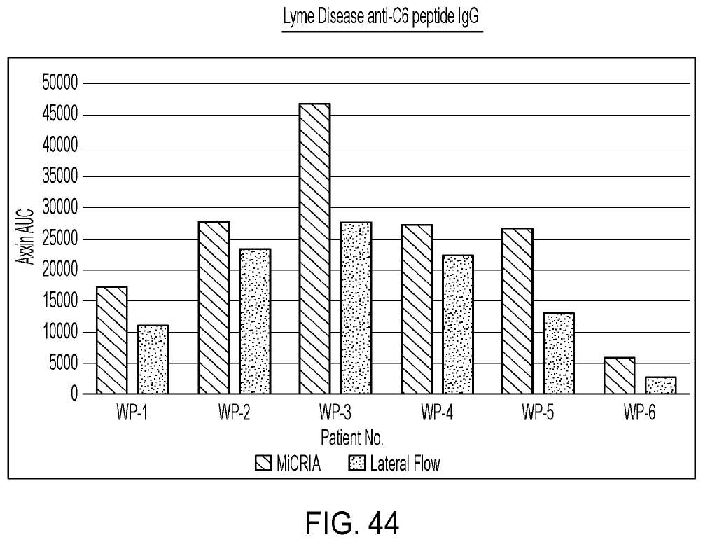

MiCRIA sensitivity was then compared to lateral flow sensitivity (FIG. 44). Lyme disease-positive (anti-C6 peptide IgG) samples were analyzed using either MiCRIA or Lateral Flow (FIG. 44). FIG. 44 demonstrates that the rapid 2-minute MiCRIA assay has greater sensitivity than a 15-minute lateral flow assay.

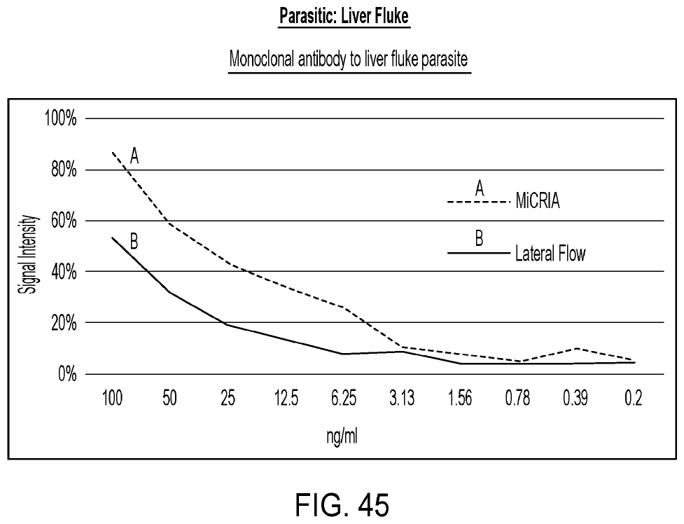

MiCRIA sensitivity was further compared to lateral flow sensitivity (FIG. 45). Liver Fluke-positive (4E11) samples were analyzed using either MiCRIA or lateral flow (FIG. 45). FIG. 45 demonstrates that the rapid 2-minute MiCRIA assay has greater sensitivity than a 15-minute lateral flow assay.

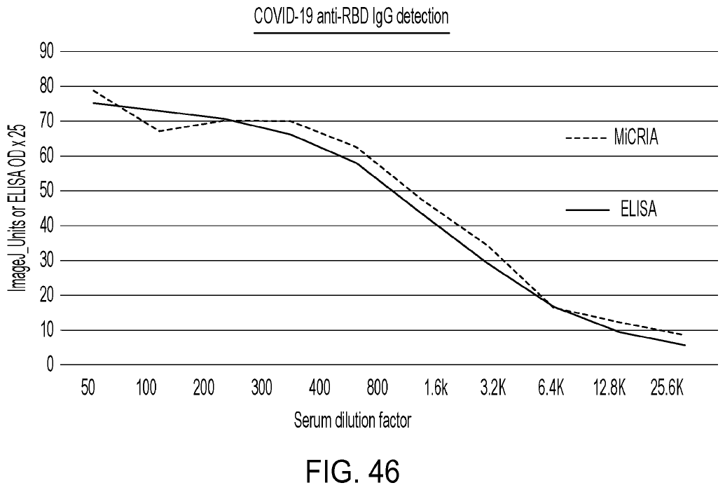

Finally, MiCRIA sensitivity was again compared to ELISA sensitivity (FIG. 46). SARS-CoV-2-positive (anti-RBD IgG) samples were analyzed using either MiCRIA or ELISA (FIG. 46). FIG. 46 demonstrates that the rapid 2-minute MiCRIA assay has comparable sensitivity to a 2-3 hour ELISA assay.

The results described in this Example (FIGS. 43-46) demonstrate that the rapid 2-minute MiCRIA assay described herein is more sensitive than the 15-minute lateral flow assay and comparable to the 2-3 hour ELISA assay while requiring significantly less complex and more readily available materials. Accordingly, MiCRIA is faster and cheaper than lateral flow and ELISA and offers better sensitivity and comparable sensitivity to lateral flow and ELISA, respectively.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### ADDITIONAL ASPECTS AND EMBODIMENTS

The following paragraphs provide additional aspects and embodiments of the present disclosure.
1. A method of detecting the presence or absence of an analyte in a sample, the method comprising:
   (i) contacting the sample with a first binding molecule bound to a detectable label and incubating the sample with the first binding molecule;
   (ii) contacting the sample with a second binding molecule bound to a magnetic particle and incubating the sample with the second binding molecule;
   (iii) adding the sample to a capillary vessel comprising an elongated capillary body having a first end and a second end disposed distal the first end, a sample entry port proximal the first end of the capillary body, and a sample exit port distal the first end of the capillary body and proximal the second end of the capillary body; and
   (iv) assessing the presence of a signal from the detectable label within the capillary vessel;
wherein:
   a magnetic field is applied to at least a portion of the capillary vessel;
   the presence of a signal within the capillary vessel is indicative of association of the analyte derived from the sample with the first binding molecule and the second binding molecule; and
   incubating the first binding molecule and the second binding molecule with the sample is sufficient to bind the first binding molecule to a first binding site on the analyte and the second binding molecule to a second binding site on the analyte.
2. A diagnostic instrument adapted to receive a capillary vessel comprising an elongated capillary body having a first end and a second end disposed distal the first end, a sample entry port proximal the first end of the capillary body, and a sample exit port distal the first end of the capillary body and proximal the second end of the capillary body;
   wherein the diagnostic instrument further comprises:
      means for application of a magnetic field to a portion of the capillary body between the sample entry port and the sample exit port; and
      means for detection of a signal in a portion of the capillary body between the sample entry port and the sample exit port.
3. A diagnostic instrument, comprising:
   a capillary vessel comprising an elongated capillary body having a first end and a second end disposed distal the first end, a sample entry port proximal the first end of the capillary body, and a sample exit port distal the first end of the capillary body and proximal the second end of the capillary body;
   means for application of a magnetic field to a portion of the capillary body between the sample entry port and the sample exit port; and
   means for detection of a signal in a portion of the capillary body between the sample entry port and the sample exit port.
4. A kit, comprising:
   a first reagent comprising a first binding molecule bound to a detectable label; and
   a second reagent comprising a second binding molecule bound to a magnetic particle.
5. A kit, comprising:
   a first reagent comprising a first binding molecule bound to a detectable label;
   a second reagent comprising a second binding molecule bound to a magnetic particle; and
   a capillary pad.
6. A kit, comprising:
   a first reagent comprising a first binding molecule bound to a detectable label;
   a second reagent comprising a second binding molecule bound to a magnetic particle; and
   a capillary vessel comprising an elongated capillary body having a first end and a second end disposed distal the first end, a sample entry port proximal the first end of the capillary body, and a sample exit port distal the first end of the capillary body and proximal the second end of the capillary body.
7. A kit, comprising:
   a first reagent comprising a detectable label adapted to be bound to a first binding molecule;
   a second reagent comprising a magnetic particle adapted to be bound to a second binding molecule;
   a capillary vessel comprising an elongated capillary body having a first end and a second end disposed distal the first end, a sample entry port proximal the first end of the capillary body, and a sample exit port distal the first end of the capillary body and proximal the second end of the capillary body.
8. A kit, comprising:
   a capillary vessel comprising an elongated capillary body having a first end and a second end disposed distal the first end, a sample entry port proximal the first end of the capillary body, and a sample exit port distal the first end of the capillary body and proximal the second end of the capillary body;
wherein:
   the capillary vessel is adapted to interface with a diagnostic instrument comprising:
      means for application of a magnetic field to a portion of the capillary body between the sample entry port and the sample exit port; and
      means for detection of a signal in a portion of the capillary body between the sample entry port and the sample exit port.
9. The method, diagnostic instrument, or kit of any one of the preceding paragraphs, wherein the analyte is an antibody.
10. The method, diagnostic instrument, or kit of any one of the preceding paragraphs, wherein the analyte is an antigen.
11. The method, diagnostic instrument, or kit of any one of the preceding paragraphs, wherein the first binding molecule is a protein.
12. The method, diagnostic instrument, or kit of any one of the preceding paragraphs, wherein the second binding molecule is a protein.
13. The method, diagnostic instrument, or kit of any one of the preceding paragraphs, wherein the first binding molecule is an antigen, an antibody, or a surface protein.
14. The method, diagnostic instrument, or kit of any one of the preceding paragraphs, wherein the second binding molecule is an antigen, an antibody, or a surface protein.
15. The method, diagnostic instrument, or kit of any one of the preceding paragraphs, wherein the magnetic particle is a magnetic bead.
16. The method, diagnostic instrument, or kit of any one of the preceding paragraphs, wherein the detectable label is Europium.
17. The method, diagnostic instrument, or kit of any one of the preceding paragraphs, wherein the first binding site is an antigen-binding site, an antibody-binding site, or an antibody Fc region.
18. The method, diagnostic instrument, or kit of any one of the preceding paragraphs, wherein the second binding site is an antigen-binding site, an antibody-binding site, or an antibody Fc region.
19. The method, diagnostic instrument, or kit of any one of the preceding paragraphs, wherein the capillary vessel is in fluid communication with a capillary pad.
20. The method of any one of the preceding paragraphs, further comprising adding a wash buffer to the capillary vessel after the sample.
21. The method of paragraph 0, wherein the sample is added to a wash buffer prior to being added to the capillary vessel.
22. The method of paragraph 0, wherein steps (i)-(v) are completed in order, simultaneously, or in any order.
23. The kit of any one of the preceding paragraphs, further comprising a third reagent comprising a wash buffer.
24. The method, diagnostic instrument, or kit of any one of the preceding paragraphs, wherein the capillary vessel is a tube.
25. The method, diagnostic instrument, or kit of any one of the preceding paragraphs, wherein the capillary vessel is substantially cylindrical.
26. The method, diagnostic instrument, or kit of any one of the preceding paragraphs, wherein the capillary vessel is a cuboid.
27. The method, diagnostic instrument, or kit of any one of the preceding paragraphs, wherein the capillary vessel comprises one or more substantially planar surfaces along its longitudinal dimension.
28. The method, diagnostic instrument, or kit of any one of the preceding paragraphs, wherein the magnetic field is generated by a magnet, or wherein the magnetic field generation means comprises a magnet.
29. The method, diagnostic instrument, or kit of any one of the preceding paragraphs, wherein the magnetic field is generated by an electromagnetic device, or wherein the magnetic field generation means is an electromagnetic device.
30. The method, diagnostic instrument, or kit of paragraph 28, wherein the magnet is a rare earth magnet.
31. The method, diagnostic instrument, or kit of paragraph 30, wherein the rare earth magnet comprises neodymium.
32. The method, diagnostic instrument, or kit of paragraph 30, wherein the rare earth magnet comprises samarium-cobalt (SmCo).
33. The diagnostic instrument of any one of the preceding paragraphs, further comprising an excitation source adapted to direct energy to a portion of the capillary body between the sample entry port and the sample exit port.
34. The method of any one of the preceding paragraphs, wherein an excitation source directs energy to a portion of the capillary body.
35. The diagnostic instrument of paragraph 33 or paragraph 34, wherein the excitation source is adapted to direct energy to an area at or near the middle of the capillary body.
36. The kit of any one of the preceding paragraphs, further comprising a capillary vessel.
37. The kit of any one of the preceding paragraphs, further comprising a capillary pad.
38. The kit of any one of the preceding paragraphs, further comprising instructions for use.
39. The method, diagnostic instrument, or kit of any one of the preceding paragraphs, wherein no reagent is bound to the capillary vessel.
40. The method, diagnostic instrument, or kit of any one of the preceding paragraphs, wherein pumping means are not used to induce the sample from the sample entry port towards the sample exit port.
41. The method, diagnostic instrument, or kit of any one of the preceding paragraphs, wherein pumping means are used to induce the sample from the sample entry port towards the sample exit port.
42. The method, diagnostic instrument, or kit of any one of the preceding paragraphs, wherein the magnetic field separates analyte bound to the first binding molecule and the second binding molecule from analyte not bound to the first binding molecule and the second binding molecule.
43. The method of paragraph 0, wherein the detectable label is detected within 100 seconds after the sample is added to the capillary vessel.
44. The method of paragraph 0, wherein the detectable label is detected approximately 10 seconds, approximately 20 seconds, approximately 30 seconds, approximately 40 seconds, approximately 50 seconds, approximately 60 seconds, approximately 70 seconds, approximately 80 seconds, approximately 90 seconds, or approximately 100 seconds after the sample is added to the capillary vessel.
45. The method of paragraph 0, wherein the detectable label is detected approximately 60 seconds after the sample is added to the capillary vessel.
46. The method of paragraph 0, wherein the detectable label is detected between approximately 10 and approximately 110 seconds, between approximately 20 and approximately 100 seconds, between approximately 30 and approximately 90 seconds, between approximately 40 and approximately 80 seconds, or between approximately 50 and approximately 70 seconds after the sample is added to the capillary vessel.
47. The diagnostic instrument of any one of the preceding paragraphs, further comprising a capillary pad adapted to be in fluid communication with the sample exit port.
48. The method or diagnostic instrument of any one of the preceding paragraphs, further comprising means for inducing a sample from the sample entry port to the sample exit port.
49. The method or diagnostic instrument of paragraph 48, wherein the means for inducing is capillary force or a pump, wherein the pump is a peristaltic pump.
50. The method or diagnostic instrument of any one of the preceding paragraphs, wherein the magnetic field is concentrated on a portion of the capillary body in or around the middle of the capillary vessel.
51. The method or diagnostic instrument of any one of the preceding paragraphs, wherein the signal is detected within the portion of the capillary body where the magnetic field is applied.
52. The method or diagnostic instrument of any one of the preceding paragraphs, wherein analytes bound to the first binding molecule and the second binding molecule are captured at the site of the magnetic field.
53. The method of paragraph 0, wherein incubating is carried out at ambient temperature.
54. The method of paragraph 0, wherein incubating is carried out at 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.
55. The method of paragraph 0, wherein incubating is carried out at 37° C.
56. The method of paragraph 0, wherein incubating is carried out at 40° C.
57. The method of paragraph 0, wherein incubating is carried out for less than 1 minute, 1 minute, 2 minutes, 3 minutes, 4 minutes, or 5 minutes.
58. The method of paragraph 0, wherein incubating is carried out for less than 1 minute.
59. The method of paragraph 0, wherein incubating is carried out for approximately 1 minute.
60. The method of paragraph 0, wherein incubating is carried out for approximately 2 minutes.
61. The method of paragraph 0, wherein incubating is carried out for approximately 3 minutes.
62. The method of paragraph 0, wherein incubating is carried out for between approximately 1 and approximately 5 minutes, between approximately 2 and approximately 4 minutes, between approximately 2 and approximately 3 minutes, or between approximately 3 and approximately 4 minutes.
63. The method, diagnostic instrument, or kit of any one of the preceding paragraphs, wherein the detectable label is a luminescent label, a colorimetric label, or a radiometric label.
64. The method, diagnostic instrument, or kit of any one of the preceding paragraphs, wherein the luminescent label is a luminescent dye, a FRET label, or a fluorescent protein.
65. The method, diagnostic instrument, or kit of any one of the preceding paragraphs, wherein the luminescent label is a lanthanide, a fluorescent label, or an organic dye.
66. The method, diagnostic instrument, or kit of any one of the preceding paragraphs, wherein the detectable label is an enzyme.
67. The method, diagnostic instrument, or kit of any one of the preceding paragraphs, wherein the detectable label is adapted to be detected by a substrate.
68. The method of any one of the preceding paragraphs, further comprising adding a substrate to the capillary vessel.
69. The diagnostic instrument or kit of any one of the preceding paragraphs, further comprising a substrate adapted to detect the detectable label.
70. The method, diagnostic instrument, or kit of any one of the preceding paragraphs, wherein the substrate is selected from the group consisting of: ABTS, OPD, AmplexRed, DAB, AEC, TMB, Homovanillic Acid, or Luminol.
71. The method, diagnostic instrument, or kit of any one of the preceding paragraphs, wherein the sample is induced from the sample entry port to the sample exit port by capillary action.
72. The method, diagnostic instrument, or kit of any one of the preceding paragraphs, wherein the sample is induced from the sample entry port to the sample exit port by pumping means.
73. The method, diagnostic instrument, or kit of any one of the preceding paragraphs, wherein the sample is induced from the sample entry port to the sample exit port by vacuuming means.
74. The method, diagnostic instrument, or kit of any one of the preceding paragraphs, wherein the sample is induced from the sample entry port to the sample exit port by airflow means.
75. The method, diagnostic instrument, or kit of any one of the preceding paragraphs, wherein the analyte is a protein, a peptide, a polypeptide, or an oligonucleotide.
76. The method, diagnostic instrument, or kit of any one of the preceding paragraphs, wherein the sample is from a subject who has or is suspected of having an infectious disease.
77. The method, diagnostic instrument, or kit of any one of the preceding paragraphs, wherein the first binding molecule and the second binding molecule are the same.
78. The method, diagnostic instrument, or kit of any one of the preceding paragraphs, wherein the first binding molecule and the second binding molecule are different.
79. The method, diagnostic instrument, or kit of any one of the preceding paragraphs, wherein the capillary vessel is enclosed in a substantially light-tight enclosure.
80. The method, diagnostic instrument, or kit of any one of the preceding paragraphs, wherein the sample is not exposed to light once it is added to the capillary vessel.
81. The method, diagnostic instrument, or kit of any one of the preceding paragraphs, further comprising a displaceable barrier in contact with the exit port.
82. The method of paragraph 0, wherein adding the sample comprises adding the sample to the sample entry point.
83. The method of paragraph 0, wherein adding the sample comprises adding the sample to a reservoir fluidically coupled to the sample entry port.
84. The method of paragraph 0, wherein capillary action does not occur immediately after the sample is added to the sample entry port.
85. The method of paragraph 0, further comprising a displaceable barrier between the sample entry port and the capillary vessel.
86. The method of paragraph 0, further comprising a displaceable barrier within the capillary vessel.
87. The method of paragraph 0, further comprising a displaceable barrier between the capillary vessel and the sample exit port.
88. The method of any one of paragraphs 85-87, wherein the displaceable barrier prevents capillary action.
89. The method of any one of the preceding paragraphs, wherein the displaceable barrier is displaced by a displacing member.
90. The method of any one of the preceding paragraphs, wherein the displaceable barrier is displaced when the capillary vessel is inserted into a reading device adapted to receive the capillary vessel.
91. The method of paragraph 90, wherein insertion of the capillary vessel into the reading device triggers a timer to trigger assessing the presence of the detectable label at a predetermined time after insertion.
92. The method of paragraph 91, wherein the predetermined time is between approximately 10-110 seconds, approximately 20-100 seconds, approximately 30-90 seconds, approximately 40-80 seconds, or approximately 50-70 seconds after insertion.
93. The diagnostic instrument or kit of any one of the preceding paragraphs, further comprising a displaceable barrier between the sample entry port and the capillary vessel.
94. The diagnostic instrument or kit of any one of the preceding paragraphs, further comprising a displaceable barrier within the capillary vessel.
95. The diagnostic instrument or kit of any one of the preceding paragraphs, further comprising a displaceable barrier between the capillary vessel and the sample exit port.
96. The diagnostic instrument or kit of any one of the preceding paragraphs, wherein the displaceable barrier is adapted to prevent capillary action.
97. The diagnostic instrument or kit of any one of the preceding paragraphs, wherein the displaceable barrier is adapted to be displaced by a displacing member.
98. The diagnostic instrument or kit of any one of the preceding paragraphs, wherein the displaceable barrier is adapted to be displaced upon insertion of the capillary vessel into a reading device adapted to receive the capillary vessel.
99. The diagnostic instrument or kit of any one of the preceding paragraphs, wherein the reading devise is adapted to trigger a timer to trigger assessment of the presence of the detectable label at a predetermined time after insertion.
100. The diagnostic instrument or kit of any one of the preceding paragraphs, wherein the predetermined time is between approximately 10-110 seconds, approximately 20-100 seconds, approximately 30-90 seconds, approximately 40-80 seconds, or approximately 50-70 seconds after insertion.
101. The diagnostic instrument of any one of the preceding paragraphs, wherein the diagnostic instrument is adapted to open to receive the capillary vessel.
102. The diagnostic instrument of any one of the preceding paragraphs, wherein the diagnostic instrument is adapted to form a substantially light-tight interior space.
103. The diagnostic instrument of any one of the preceding paragraphs, further comprising a displacing member.
104. The method, diagnostic instrument, or kit of any one of the preceding paragraphs, wherein the sample is a biological sample.
105. The method, diagnostic instrument, or kit of any one of the preceding paragraphs, wherein the biological sample is selected from the group consisting of: saliva, oral fluid, tears, urine, interstitial fluid, synovial fluid, nasal fluid, nasopharyngeal fluid, and cerebrospinal fluid.
106. The method, diagnostic instrument, or kit of any one of the preceding paragraphs, wherein the biological sample is blood.
107. The method, diagnostic instrument, or kit of any one of the preceding paragraphs, wherein the biological sample is blood, whole blood, dried blood, serum, plasma, or a blood fraction.
108. The method, diagnostic instrument, or kit of any one of the preceding paragraphs, wherein the sample is collected from a subject.
109. The method, diagnostic instrument, or kit of any one of the preceding paragraphs, wherein the blood is collected from a subject suspected of having or who has an infectious disease.
110. The method, diagnostic instrument, or kit of any one of the preceding paragraphs, wherein the infectious disease is Lyme disease, human immunodeficiency virus, or SARS-CoV-2.
111. The method, diagnostic instrument, or kit of any one of the preceding paragraphs, wherein the subject is a human or a non-human animal.
112. The method, diagnostic instrument, or kit of any one of the preceding paragraphs, wherein the subject is human.
113. The method, diagnostic instrument, or kit of any one of the preceding paragraphs, wherein the sample is collected from a subject suspected of having a disease caused by a pathogenic member of the bacterial genus Borrelia.
114. The method, diagnostic instrument, or kit of any one of the preceding paragraphs, wherein the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica.
115. The method, diagnostic instrument, or kit of any one of the preceding paragraphs, wherein the pathogenic member of the bacterial genus Borrelia is Borrelia burgdorferi.
116. A housing assembly, comprising:
   a top portion; and
   a bottom portion removably attached to the top portion;
   wherein:
      the top portion is adapted to receive one or more cameras and one or more excitation sources;
      the bottom portion is adapted to receive a magnet; and
      the bottom portion is adapted to receive a diagnostic instrument.
117. The housing assembly of paragraph Error! Reference source not found., wherein the housing assembly encloses an interior space and is adapted to enclose at least a portion of the diagnostic instrument in the interior space of the housing assembly.
118. The housing assembly of any one of the preceding paragraphs, wherein the diagnostic instrument comprises a reservoir disposed on the exterior of the housing assembly when the diagnostic instrument is received in the housing assembly.
119. The housing assembly of any one of the preceding paragraphs, wherein the diagnostic instrument comprises a capillary body disposed on the interior of the housing assembly when the diagnostic instrument is received in the housing assembly.
120. The housing assembly of any one of the preceding paragraphs, wherein the diagnostic instrument is the diagnostic instrument of any one of the preceding paragraphs.
121. The housing assembly of any one of the preceding paragraphs, wherein the housing assembly is adapted to facilitate the method of any one of the preceding paragraphs.
122. The housing assembly of any one of the preceding paragraphs, wherein the housing assembly is adapted to receive the kit of any one of the preceding paragraphs.
123. The housing assembly of any one of the preceding paragraphs, wherein the one or more cameras are affixed to the top portion.
124. The housing assembly of any one of the preceding paragraphs, wherein the one or more excitation sources are affixed to top portion.
125. The housing assembly of any one of the preceding paragraphs, wherein the magnet is affixed to the bottom portion.
126. The housing assembly of any one of the preceding paragraphs, wherein the housing assembly is battery-powered.
127. The housing assembly of any one of the preceding paragraphs, wherein the housing assembly is powered by an external power source.
128. The housing assembly of any one of the preceding paragraphs, wherein the housing assembly is operably engaged by the addition of liquid into the diagnostic instrument.
129. The housing assembly of any one of the preceding paragraphs, further comprising a switch.
130. The housing assembly of any one of the preceding paragraphs, wherein the switch is togglable.
131. The housing assembly of any one of the preceding paragraphs, wherein operating the switch operably engages the one or more cameras and the one or more excitation sources.
132. The housing assembly of any one of the preceding paragraphs, wherein operating the switch initiates flow of liquid through the diagnostic instrument.
133. The housing assembly of any one of the preceding paragraphs, wherein attaching the top portion to the bottom portion creates a substantially light-tight seal.

### REFERENCES

1. Juntunen E, Myyryläinen T, Salminen T, Soukka T, Pettersson K. Performance of fluorescent europium(III) nanoparticles and colloidal gold reporters in lateral flow bioaffinity assay. Anal Biochem 428(1):31-8 (2012).
2. Luo K, Hu L, Guo Q, Wu C, Wu S, Liu D, et al. Comparison of 4 label-based immunochromatographic assays for the detection of Escherichia coli O157:H7 in milk. J Dairy Sci 100(7):5176-87 (2017).
3. Morrissey D V., Lombardo M, Eldredge J K, Kearney K R, Groody E P, Collins M L. Nucleic acid hybridization assays employing dA-tailed capture probes: I. Multiple capture methods. Anal Biochem 181(2):345-59 (1989).
4. Bach H J, Hartmann A, Trevors J T, Munch J C. Magnetic capture-hybridization method for purification and probing of mRNA for neutral protease of Bacillus cereus. J Microbiol Methods 37(2):187-92 (1999).
5. Chandler L J, Blair C D, Beaty B J. Detection of Dengue-2 Viral RNA by Reversible Target Capture Hybridization. J Clin Microbiol. 31(10):2641-2647 (1993).
6. Fluorophore selection. Thermo Fisher Scientific.
7. Handali S, Klarman M, Gaspard A N, Dong X F, Laborde R, Noh J, et al. Development and evaluation of a magnetic immunochromatographic test to detect Taenia solium, which causes taeniasis and neurocysticercosis in humans. Clin Vaccine Immunol. 17(4):631-7 (2010).
8. Burbelo P D, Gunti S, Keller J M, Morse C G, Deeks S G, Lionakis M S, et al. Ultrarapid Measurement of Diagnostic Antibodies by Magnetic Capture of Immune Complexes. Sci Rep. 7(1):1-11 (2017).
9. Kim C, Hoffmann G, Searson P C. Integrated Magnetic Bead—Quantum Dot Immunoassay for Malaria Detection. ACS Sensors 2(6):766-72 (2017).
10. Mechaly A, Marx S, Levy O, Yitzhaki S, Fisher M. Highly Stable Lyophilized Homogeneous Bead-Based Immunoassays for On-Site Detection of Bio Warfare Agents from Complex Matrices. Anal Chem 88(12):6283-91 (2016).
11. Crosslinking Technology: Reactivity chemistries, applications and structure references. Thermo Fisher Scientific.
12. Crosslinker Selection Guide. Thermo Fisher Scientific.
13. Fischer M J E. Amine Coupling Through EDC/NHS: A Practical Approach. Methods Mol Biol. 627:55-73 (2010).
14. Covalent Coupling. Thermo Fisher Scientific.
15. Dorn J, Masciotra S, Yang C, Downing R, Biryahwaho B, Mastro T D, et al. Analysis of genetic variability within the immunodominant epitopes of envelope gp41 from human immunodeficiency virus type 1 (HIV-1) group M and its impact on HIV-1 antibody detection. J Clin Microbiol. 38(2):773-80 (2000).
16. Wei X, Liu X, Dobbs T, Kuehl D, Nkengasong J N, Hu D J, et al. Development of Two Avidity-Based Assays to Detect Recent HIV Type 1 Seroconversion Using a Multisubtype gp41 Recombinant Protein. AIDS Res Hum Retroviruses 26(1):61-71 (2010).
17. Fluoro-Max™ Fluorescent Carboxylate-Modified Particles. ThermoFisher Scientific.
18. Sera-Mag SpeedBeads and Sera-Mag Carboxylate-Modified Magnetic Particles—GE Healthcare Life Sciences.
19. Ting Liang F, Steere A C, Marques A R, B Johnson B J, Miller J N, Philipp M T. Sensitive and Specific Serodiagnosis of Lyme Disease by Enzyme-Linked Immunosorbent Assay with a Peptide Based on an Immunodominant Conserved Region of Borrelia burgdorferi VlsE. J Clin Microbiol. 37(12):3990-6 (1999).
20. Wormser G P, Schriefer M, Aguero-Rosenfeld M E, Levin A, Steere A C, Nadelman R B, et al. Single-tier testing with the C6 peptide ELISA kit compared with two-tier testing for Lyme disease. Diagn Microbiol Infect Dis. 75(1):9-15 (2013).
21. Wormser G P, Nowakowski J, Nadelman R B, Visintainer P, Levin A, Aguero-Rosenfeld M E. Impact of clinical variables on Borrelia burgdorferi-specific antibody seropositivity in acute-phase sera from patients in North America with culture-confirmed early Lyme disease. Clin Vaccine Immunol. 15(10):1519-22 (2008).
22. Bacon R M, Biggerstaff B J, Schriefer M E, Gilmore, Jr. R D, Philipp M T, Steere A C, et al. Serodiagnosis of Lyme Disease by Kinetic Enzyme-Linked Immunosorbent Assay Using Recombinant VlsE1 or Peptide Antigens of Borrelia burgdorferi Compared with 2-Tiered Testing Using Whole-Cell Lysates. J Infect Dis. 187:1187-99 (2003).
23. Sugden J. Photochemistry of dyes and fluorochromes used in biology and medicine: some physicochemical background and current applications. Biotech Histochem 79(2):71-90 (2004).
24. Herman B. Absorption and Emission Maxima for Common Fluorophores. Curr Protoc Cell Biol 00(1):A.1E.1-A.1E.5 (1998).
25. Adegoke O, Park E Y. The use of nanocrystal quantum dot as fluorophore reporters in molecular beacon-based assays. Nano Converg 3(1):32(2016 ).
26. Goldman E R, Mattoussi H, Anderson G P, Medintz I L, Mauro J M. Fluoroimmunoassays Using Antibody-Conjugated Quantum Dots. In: NanoBiotechnology Protocols. Methods Mol Biol. 303:19-34 (2005).
27. Rong Y, Wu C, Yu J, Zhang X, Ye F, Zeigler M, et al. Multicolor Fluorescent Semiconducting Polymer Dots with Narrow Emissions and High Brightness. ACS Nano 7(1):376-84 (2013).
28. Wu C, Bull B, Szymanski C, Christensen K, McNeill J. Multicolor Conjugated Polymer Dots for Biological Fluorescence Imaging. ACS Nano (11):2415-23 (2008).
29. Qdot Nanocrystals—Section 6.6. Thermo Fisher Scientific.
30. Fluorescence Imaging with Quantum Dot Nanocrystals. Semrock.
31. Spadaro, A. M. Lyophilization of reagent-coated particles. (U.S. Pat. No. 4,693,912).
32. Abdelwahed W, Degobert G, Stainmesse S, Fessi H. Freeze-drying of nanoparticles: Formulation, process and storage considerations. Adv. Drug Deliv Rev. 58(15):1688-1713 (2006).
33. Day J G, Stacey G (Glyn). Cryopreservation and freeze-drying protocols. Humana Press 347 (2007 ).
34. Challener C A. For Lyophilization, Excipients Really Do Matter. BipPharm International 30(1):32-35 (2017).
35. Anderson G. Determination of Product Shelf Life and Activation Energy for Five Drugs of Abuse. Clin Chem 37(3):398 (1991).
36. Magari R T, Murphy K P, Fernandez T. Accelerated stability model for predicting shelf-life. J Clin Lab Anal. 16(5):221-6 (2002).
37. Target product profiles (TPPs) for diagnostic tests. https://www.finddx.org/target-product-profiles/
38. Porrás A I, Yadon Z E, Altcheh J, Britto C, Chaves G C, Flevaud L, et al. Target product profile (TPP) for chagas disease point-of-care diagnosis and assessment of response to treatment. PLoS Negl Trop Dis. 9(6):1-8 (2015).
39. Donadeu M, Fahrion A S, Olliaro P L, Abela-Ridder B. Target product profiles for the diagnosis of Taenia solium taeniasis, neurocysticercosis and porcine cysticercosis. PLoS Negl Trop Dis. 11(9):1-18 (2017).
40. Dittrich S, Tadesse B T, Moussy F, Chua A, Zorzet A, Tängdén T, et al. Target Product Profile for a Diagnostic Assay to Differentiate between Bacterial and Non-Bacterial Infections and Reduce Antimicrobial Overuse in Resource-Limited Settings: An Expert Consensus. PLoS One 11(8):e0161721 (2016).
41. Wagar E A, Yasin B, Yuan S. Point-of-Care Testing: Twenty Years' Experience. Lab Med 39(9):560-3 (2008).
42. Jauset-Rubio M, Svobodová M, Mairal T, McNeil C, Keegan N, Saeed A, et al. Ultrasensitive, rapid and inexpensive detection of DNA using paper based lateral flow assay. Sci Rep. 6(1):37732 (2016).
43. Larson B, Schnippel K, Ndibongo B, Long L, Fox M P, Rosen S. How to Estimate the Cost of Point-of-Care CD4 Testing in Program Settings: An Example Using the Alere Pima™ Analyzer in South Africa. PLoS One 7(4):e35444 (2012).
44. Hortin G L. Special Report Does Point-Of-Care Testing Save Money or Cost More? (2018)
45. Burbelo, P. (2020). Method and device for detecting antigen-specific antibodies in a biological fluid sample using neodymium magnets (U.S. Pat. No. 10,564,152)
46. Kim C, Hoffmann G, Searson P C. Integrated Magnetic Bead-Quantum Dot Immunoassay for Malaria Detection. ACS Sensors 2(6):766-72 (2017).
47. Mechaly A, Marx S, Levy O, Yitzhaki S, Fisher M. Highly Stable Lyophilized Homogeneous Bead-Based Immunoassays for On-Site Detection of Bio Warfare Agents from Complex Matrices. Anal Chem 88(12):6283-91 (2016).
48. Lowe, P. (2019). Assay device and reader (U.S. Pat. No. 10,376,881)

What is claimed is:

1. A method of detecting the presence or absence of an analyte in a sample, the method comprising:
(i) contacting the sample with a first binding molecule bound to a detectable label and incubating the sample with the first binding molecule;
(ii) contacting the sample with a second binding molecule bound to a magnetic particle and incubating the sample with the second binding molecule;
(iii) adding the sample to a capillary vessel comprising an elongated capillary body having a first end and a second end disposed distal the first end, a sample entry port proximal the first end of the capillary body, and a sample exit port distal the first end of the capillary body and proximal the second end of the capillary body; and
(iv) assessing the presence of a signal from the detectable label within the capillary vessel;
wherein:
a magnetic field is applied to at least a portion of the capillary vessel;
the presence of a signal within the capillary vessel is indicative of association of the analyte derived from the sample with the first binding molecule and the second binding molecule; and
incubating the first binding molecule and the second binding molecule with the sample is sufficient to bind the first binding molecule to a first binding site on the analyte and the second binding molecule to a second binding site on the analyte.

2. The method of any one of the preceding claims, wherein the analyte is an antibody or an antigen.

3. The method of any one of the preceding claims, wherein at least one of the first and the second binding molecule is a protein.

4. The method of any one of the preceding claims, wherein at least one of the first and the second binding molecule is an antigen, an antibody, or a surface protein.

5. The method of any one of the preceding claims, wherein the magnetic particle is a magnetic bead.

6. The method of any one of the preceding claims, wherein the detectable label is Europium.

7. The method of claim 1, wherein steps (i)-(v) are completed in order, simultaneously, or in any order.

8. The method of any one of the preceding claims, wherein the magnetic field is generated by a magnet or an electromagnetic device, or wherein the magnetic field generation means comprises a magnet or is an electromagnetic device.

9. The method of claim 8, wherein the magnet is a rare earth magnet, wherein the rare earth magnet comprises neodymium or samarium-cobalt (SmCo).

10. The method of any one of the preceding claims, wherein the magnetic field separates analyte bound to the first binding molecule and the second binding molecule from analyte not bound to the first binding molecule and the second binding molecule.

11. The method of claim 1, wherein the detectable label is detected between approximately 10 and approximately 110 seconds, between approximately 20 and approximately 100 seconds, between approximately 30 and approximately 90 seconds, between approximately 40 and approximately 80 seconds, or between approximately 50 and approximately 70 seconds after the sample is added to the capillary vessel.

12. The method of any one of the preceding claims, wherein the detectable label is a luminescent label, a colorimetric label, or a radiometric label.

13. The method of any one of the preceding claims, further comprising adding a substrate to the capillary vessel.

14. The method of any one of the preceding claims, wherein the substrate is selected from the group consisting of: ABTS, OPD, AmplexRed, DAB, AEC, TMB, Homovanillic Acid, or Luminol.

15. The method of any one of the preceding claims, wherein the sample is induced from the sample entry port to the sample exit port by capillary action, pumping means, vacuuming means, or airflow means.

16. The method of any one of the preceding claims, wherein the analyte is a protein, a peptide, a polypeptide, or an oligonucleotide.

17. The method of any one of the preceding claims, wherein the sample is obtained from a subject who has or is suspected of having an infectious disease.

18. The method of any one of the preceding claims, wherein the sample is a biological sample selected from the group consisting of: saliva, oral fluid, tears, urine, interstitial fluid, synovial fluid, nasal fluid, nasopharyngeal fluid, and cerebrospinal fluid.

19. The method of any one of the preceding claims, wherein the infectious disease is Lyme disease, human immunodeficiency virus, or SARS-CoV-2.

20. The method of any one of the preceding claims, wherein the sample is collected from a subject suspected of having a disease caused by a pathogenic member of the bacterial genus Borrelia, wherein the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica.

21. A diagnostic instrument adapted to receive a capillary vessel comprising an elongated capillary body having a first end and a second end disposed distal the first end, a sample entry port proximal the first end of the capillary body, and a sample exit port distal the first end of the capillary body and proximal the second end of the capillary body;
wherein the diagnostic instrument further comprises:
means for application of a magnetic field to a portion of the capillary body between the sample entry port and the sample exit port; and
means for detection of a signal in a portion of the capillary body between the sample entry port and the sample exit port.

22. A diagnostic instrument, comprising:
a capillary vessel comprising an elongated capillary body having a first end and a second end disposed distal the first end, a sample entry port proximal the first end of the capillary body, and a sample exit port distal the first end of the capillary body and proximal the second end of the capillary body;
means for application of a magnetic field to a portion of the capillary body between the sample entry port and the sample exit port; and
means for detection of a signal in a portion of the capillary body between the sample entry port and the sample exit port.

23. The diagnostic instrument of any one of the preceding claims, wherein the analyte is an antibody or an antigen.

24. The diagnostic instrument of any one of the preceding claims, wherein the magnetic particle is a magnetic bead.

25. The diagnostic instrument of any one of the preceding claims, wherein the detectable label is Europium.

26. The diagnostic instrument of any one of the preceding claims, wherein the capillary vessel is in fluid communication with a capillary pad.

27. The diagnostic instrument of any one of the preceding claims, wherein the magnetic field is generated by a magnet, or wherein the magnetic field generation means comprises a magnet.

28. The diagnostic instrument of any one of the preceding claims, wherein the magnetic field is generated by an electromagnetic device, or wherein the magnetic field generation means is an electromagnetic device.

29. The diagnostic instrument of any one of the preceding claims, further comprising an excitation source adapted to direct energy to a portion of the capillary body between the sample entry port and the sample exit port.

30. The diagnostic instrument of claim 29, wherein the excitation source is adapted to direct energy to an area at or near the middle of the capillary body.

31. The diagnostic instrument of any one of the preceding claims, wherein the magnetic field separates analyte bound to the first binding molecule and the second binding molecule from analyte not bound to the first binding molecule and the second binding molecule.

32. The diagnostic instrument of any one of the preceding claims, further comprising a capillary pad adapted to be in fluid communication with the sample exit port.

33. The diagnostic instrument of any one of the preceding claims, further comprising means for inducing a sample from the sample entry port to the sample exit port.

34. The diagnostic instrument of claim 33, wherein the means for inducing is capillary force or a pump, wherein the pump is a peristaltic pump.

35. The diagnostic instrument of any one of the preceding claims, wherein the magnetic field is concentrated on a portion of the capillary body in or around the middle of the capillary vessel.

36. The diagnostic instrument of any one of the preceding claims, wherein the signal is detected within the portion of the capillary body where the magnetic field is applied.

37. The diagnostic instrument of any one of the preceding claims, wherein analytes bound to the first binding molecule and the second binding molecule are captured at the site of the magnetic field.

38. The diagnostic instrument any one of the preceding claims, wherein the detectable label is a luminescent label, a colorimetric label, or a radiometric label.

39. The diagnostic instrument of any one of the preceding claims, wherein the diagnostic instrument is adapted to open to receive the capillary vessel.

40. The diagnostic instrument of any one of the preceding claims, wherein the diagnostic instrument is adapted to form a substantially light-tight interior space.

41. A kit, comprising:
a first reagent comprising a first binding molecule bound to a detectable label; and
a second reagent comprising a second binding molecule bound to a magnetic particle.

42. A kit, comprising:
a first reagent comprising a first binding molecule bound to a detectable label;
a second reagent comprising a second binding molecule bound to a magnetic particle; and
a capillary pad.

43. A kit, comprising:
a first reagent comprising a first binding molecule bound to a detectable label;
a second reagent comprising a second binding molecule bound to a magnetic particle; and
a capillary vessel comprising an elongated capillary body having a first end and a second end disposed distal the first end, a sample entry port proximal the first end of the capillary body, and a sample exit port distal the first end of the capillary body and proximal the second end of the capillary body.

44. A kit, comprising:
a first reagent comprising a detectable label adapted to be bound to a first binding molecule;
a second reagent comprising a magnetic particle adapted to be bound to a second binding molecule;
a capillary vessel comprising an elongated capillary body having a first end and a second end disposed distal the first end, a sample entry port proximal the first end of the capillary body, and a sample exit port distal the first end of the capillary body and proximal the second end of the capillary body.

45. A kit, comprising:
a capillary vessel comprising an elongated capillary body having a first end and a second end disposed distal the first end, a sample entry port proximal the first end of the capillary body, and a sample exit port distal the first end of the capillary body and proximal the second end of the capillary body;
wherein:
the capillary vessel is adapted to interface with a diagnostic instrument comprising:
means for application of a magnetic field to a portion of the capillary body between the sample entry port and the sample exit port; and
means for detection of a signal in a portion of the capillary body between the sample entry port and the sample exit port.

46. The kit of any one of the preceding claims, further comprising a third reagent comprising a wash buffer, a capillary vessel, a capillary pad, and/or instructions for use.

47. A housing assembly, comprising:
a top portion; and
a bottom portion removably attached to the top portion;
wherein:
the top portion is adapted to receive one or more cameras and one or more excitation sources;
the bottom portion is adapted to receive a magnet; and
the bottom portion is adapted to receive a diagnostic instrument.

48. The housing assembly of claim 47, wherein the housing assembly encloses an interior space and is adapted to enclose at least a portion of the diagnostic instrument in the interior space of the housing assembly.

49. The housing assembly of any one of the preceding claims, wherein the diagnostic instrument comprises a reservoir disposed on the exterior of the housing assembly when the diagnostic instrument is received in the housing assembly.

50. The housing assembly of any one of the preceding claims, wherein the diagnostic instrument comprises a capillary body disposed on the interior of the housing assembly when the diagnostic instrument is received in the housing assembly.

51. The housing assembly of any one of the preceding claims, wherein the diagnostic instrument is the diagnostic instrument of any one of the preceding claims.

52. The housing assembly of any one of the preceding claims, wherein the one or more cameras are affixed to the top portion.

53. The housing assembly of any one of the preceding claims, wherein the one or more excitation sources are affixed to top portion.

54. The housing assembly of any one of the preceding claims, wherein the magnet is affixed to the bottom portion.

55. The housing assembly of any one of the preceding claims, wherein the housing assembly is operably engaged by the addition of liquid into the diagnostic instrument.

56. The housing assembly of any one of the preceding claims, further comprising a toggleable switch, wherein operating the switch operably engages the one or more camera and the one or more excitations source and initiates flow of liquid through the diagnostic instrument.

57. The housing assembly of any one of the preceding claims, wherein attaching the top portion to the bottom portion creates a substantially light-tight seal.
